(19) United States
(12) Patent Application Publication
(10) Pub. No.: US 2005/0043358 A1
(43) Pub. Date: Feb. 24, 2005

(54) Aminoalcohol derivatives and their use as beta 3 adrenergic agonists

(75) Inventors: Kiyoshi Taniguchi, Hyogo (JP); Minoru Sakurai, Osaka (JP); Takeshi Kato, Hyogo (JP); Naoaki Fujii, Osaka (JP); Kenichi Washizuka, Osaka (JP); Yasuyo Tomishima, Osaka (JP); Hisashi Takasugi, Osaka (JP); Yutaka Kohno, Osaka (JP); Nobuhiro Yamamoto, Osaka (JP); Naoko Unami, Hyogo (JP); Hirohumi Ishikawa, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co. Ltd., Osaka-shi (JP)

(21) Appl. No.: 10/746,267

(22) Filed: Dec. 29, 2003

Related U.S. Application Data
(63) Continuation of application No. 10/118,929, filed on Apr. 10, 2002, now abandoned.
Application No. 10/118,929 is a continuation of application No. 09/868,615, filed on Jun. 22, 2001, now abandoned.

(30) Foreign Application Priority Data
Dec. 20, 1998 (AU) PP7967

(51) Int. Cl. A61K 31/495; C07D 213/54; C07D 213/24

(52) U.S. Cl. 514/332; 514/340; 514/357; 514/651; 546/268.1; 546/264; 546/334

(57) ABSTRACT

A compound of the formula (I): 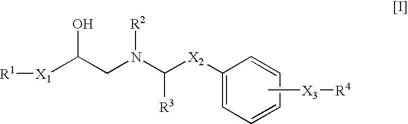
wherein X₁ is bond or —OCH₂—;
X₂ is —(CH₂)ₙ—, in which n is 1, 2 or 3;
X₃ is bond, —O—, —S—, —OCH₂—, or —NH—;
R¹ is phenyl or pyridyl each of which may have one or two substituent(s) selected from the group consisting of hydroxy, halogen, etc.;
R² is hydrogen, (lower)alkoxycarbonyl, etc.;
R³ is hydroxy(lower)alkyl; halo(lower)alkyl, etc.;
R⁴ is aryl or unsaturated heterocyclic group, each of which may have one or two substituent(s) selected from the group consisting of lower alkyl, hydroxy, carbamoyl, halogen, lower alkoxy, etc.; and a salt thereof which is useful as a medicament.

### TECHNICAL FIELD

This invention relates to new aminoalcohol derivatives and salts thereof which are useful as a medicament.

### DISCLOSURE OF INVENTION

This invention relates to new aminoalcohol derivatives and salts thereof.

More particularly, it relates to new aminoalcohol derivatives and salts thereof which act as selective β₃ adrenergic receptor agonists and therefore have gut selective sympathomimetic, anti-ulcerous, anti-pancreatitis, lipolytic, anti-urinary incontinence and anti-pollakiuria activities, to processes for the preparation thereof, to a pharmaceutical composition comprising the same and to a method of using the same therapeutically in the treatment and/or prevention of gastrointestinal disorders caused by smooth muscle contractions in human beings or animals, and more particularly to a method for the treatment and/or prevention of spasm or hyperanakinesia in case of irritable bowel syndrome, gastritis, gastric ulcer, duodenal ulcer, enteritis, cholecystopathy, cholangitis, urinary calculus and the like; for the treatment and/or prevention of ulcer such as gastric ulcer, duodenal ulcer, peptic ulcer, ulcer caused by non steroidal anti-inflammatory drugs, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis or the like; for the treatment and/or prevention of pancreatitis, obesity, diabetes, glycosuria, hyperlipidemia, hypertension, atherosclerosis, glaucoma, melancholia, depression and the like, and for the treatment and/or prevention of a wasting condition, weight loss, emarciation or the like.

One object of this invention is to provide new and useful aminoalcohol derivatives and salts thereof which have gut selective sympathomimetic, anti-ulcerous, lipolytic, anti-urinary incontinence and anti-pollakiuria activities.

Another object of this invention is to provide processes for the preparation of said aminoalcohol derivatives and salts thereof.

A further object of this invention is to provide a pharmaceutical composition comprising, as an active ingredient, said aminoalcohol derivatives and salts thereof.

Still further object of this invention is to provide a therapeutical method for the treatment and/or prevention of aforesaid diseases in human beings or animals, using said aminoalcohol derivatives and salts thereof.

The object aminoalcohol derivatives of this invention are new and can be represented by the following general formula [I]: 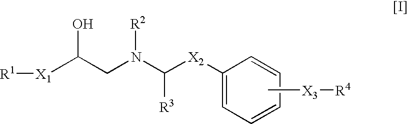
wherein
X₁ is bond or —OCH₂—;
X₂ is —(CH₂)ₙ—, in which n is 1, 2 or 3;
X₃ is bond, —O—, —S—, —OCH₂— or —NH—;
R¹ is phenyl or pyridyl, each of which may be substituted with one or two substituent(s) selected from the group consisting of hydroxy, halogen, amino, [(lower)alkylsulfonyl]amino, nitro, benzyloxycarbonylamino and benzyloxy;
R² is hydrogen, (lower)alkoxycarbonyl, benzyl or benzyloxycarbonyl;
R³ is hydroxy(lower)alkyl, (lower)alkoxy(lower)alkyl or halo(lower)alkyl; and
R⁴ is aryl or an unsaturated heterocyclic group containing nitrogen, each of which may be substituted with one or two substituent(s) selected from the group consisting of hydroxy, lower alkyl, lower alkoxy, halo(lower)alkyl, halogen, hydroxy(lower)alkyl, (lower)alkoxy(lower)alkyl, cyano, carboxy, (lower)alkoxycarbonyl, lower alkanoyl, carbamoyl, (mono or di)(lower)-alkylcarbamoyl, [(lower)alkylsulfonyl]carbamoyl, amino, nitro, ureido, [(lower)alkylcarbonyl]amino, [(lower)alkylsulfonyl]amino and (arylsulfonyl)amino,
and a salt thereof.

The object compound [I] or a salt thereof can be prepared by the following processes. 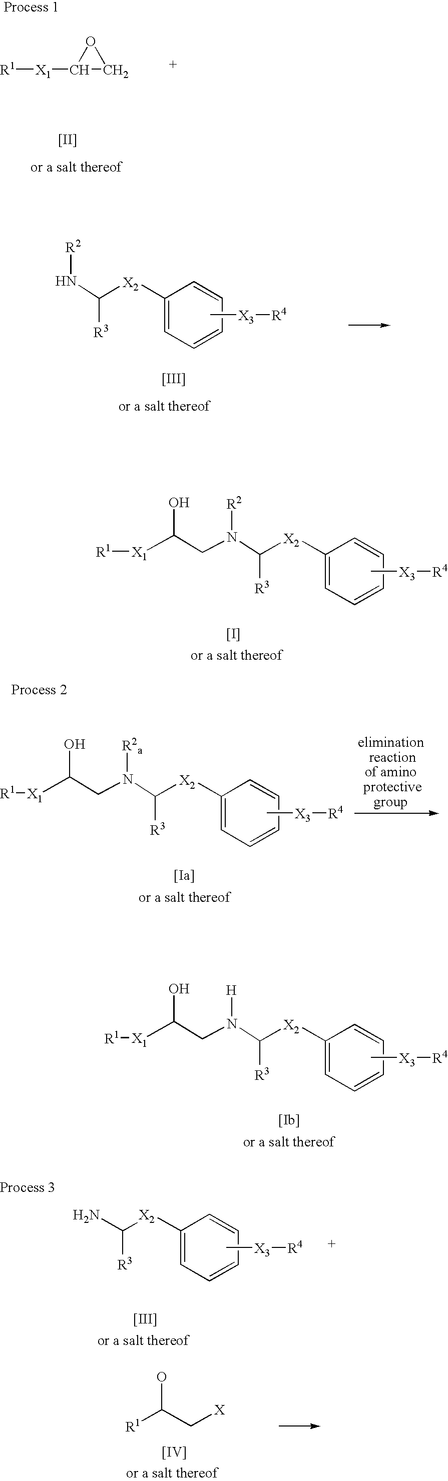 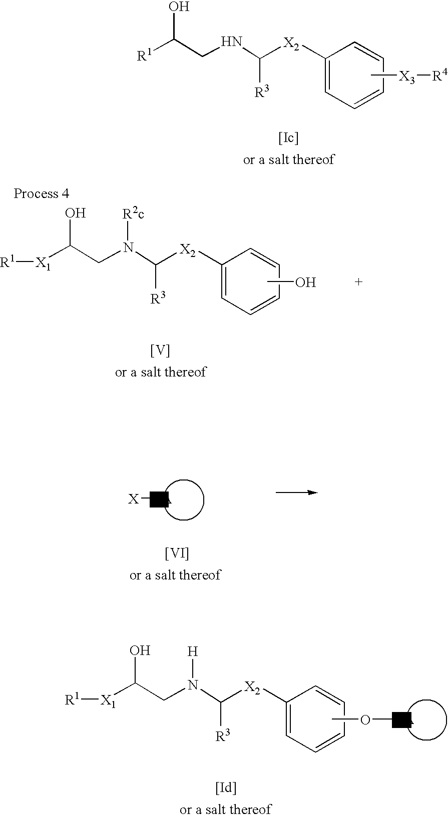
wherein X₁, X₂, X₃, R¹, R², R³ and R⁴ are each as defined above,
Rₐ² and R_{c}² are each amino protective group,
Q is protected hydroxy,
X is halogen, and 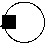
is aryl or an unsaturated heterocyclic group containing nitrogen, each of which may be substituted with one or two substituent(s) selected from the group consisting of hydroxy, lower alkyl, lower alkoxy, halo(lower)alkyl, halogen, hydroxy(lower)alkyl, (lower)alkoxycarbonyl, lower alkanoyl, carbamoyl, (mono or di)(lower)-alkylcarbamoyl, [(lower)alkylsulfonyl]carbamoyl, amino, nitro, ureido, [(lower)alkylcarbamoyl]amino, [(lower)alkylsulfonyl]carbamoyl and (arylsulfonyl)amino.

In the above and subsequent description of the present specification, suitable examples of the various definition to be included within the scope of the invention are explained in detail in the following.

The term “lower” is intended to mean a group having 1 to 6 carbon atom(s), unless otherwise provided.

Suitable “lower alkyl” and “lower alkyl” moiety in the terms of “[(lower)alkylsulfonyl]amino”, “hydroxy(lower)-alkyl”, etc. may include straight or branched one having 1 to 6 carbon atom(s), such as methyl, ethyl, propyl, isopropyl, butyl, isobutyl, sec-butyl, tert-butyl, pentyl, 1-methylpentyl, tert-pentyl, neo-pentyl, hexyl, isohexyl and the like.

Suitable “lower alkoxy” and “lower alkoxy” moiety in the terms of “(lower)alkoxycarbonyl”, “(lower)alkoxy(lower)-alkyl”, etc. may be a straight or branched one such as methoxy, ethoxy, propoxy, isopropoxy, 1-ethylpropoxy, butoxy, sec-butoxy, tert-butoxy, pentyloxy, neopentyloxy, tert-pentyloxy, hexyloxy, and the like, in which the preferred one may be C₁-C₄ alkoxy, and the most preferred one may be methoxy.

Suitable “lower alkanoyl” may include formyl, acetyl, propanoyl, butanoyl, 2-methylpropanoyl, pentanoyl, 2,2-dimethylpropanoyl, hexanoyl and the like.

Suitable “halogen” may be fluoro, chloro, bromo and iodo.

Suitable “aryl” and “aryl” moiety in the term of “(arylsulfonyl)amino” may include phenyl, naphthyl, anthryl, and the like, in which the preferred one may be phenyl.

Suitable “an unsaturated heterocyclic group containing nitrogen” may include an unsaturated, monocyclic or polycyclic heterocyclic group containing at least one nitrogen atom. And especially preferable unsaturated heterocyclic group containing nitrogen may be ones such as
an unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl and its N-oxide, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g., 1H-tetrazolyl, 2H-tetrazolyl, etc.), etc.;
an unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, etc.; and the like.

Suitable-“hydroxy protective group” in the term “protected hydroxy” may include commonly protective group or the like.

Suitable common protective group may include acyl as mentioned below, mono(or di or tri)phenyl(lower)alkyl which may have one or more suitable substituent(s) (e.g. benzyl, 4-methoxyphenyl, trityl, etc.), trisubstituented silyl [e.g., tri(lower)alkylsilyl (e.g., trimethylsilyl; t-butyldimethylsilyl, etc.], tetrahydropyranyl and the like.

Suitable “acyl” may include carbamoyl, aliphatic acyl group and acyl group containing an aromatic ring, which is referred to as aromatic acyl, or heterocyclic ring, which is referred to as heterocyclic acyl.

Suitable examples of said acyl may be illustrated as follows;

carbamoyl; carboxy; aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.); cyclo(lower)alkylcarbonyl (e.g., cyclopropylcarbonyl, cyclobutylcarbonyl, cyclopentylcarbonyl, cyclohexylcarbonyl, cyclopentylcarbonyl, etc.), protected carboxy such as commonly protected carboxy [e.g., esterified carboxy such as lower or higher alkoxycarbonyl (e.g., methoxycarbonyl, ethoxycarbonyl, propyloxycarbonyl, iso-propyloxycarbonyl, t-butoxycarbonyl, t-pentyloxycarbonyl, heptyloxycarbonyl, etc.] or the like; lower or higher alkylsulfonyl (e.g., methylsulfonyl, ethylsulfonyl, etc.); lower or higher alkoxysulfonyl (e.g., methoxysulfonyl, ethoxysulfonyl, etc.); di(lower)alkoxyphosphoryl (e.g., dimethoxyphosphoryl, diethoxyphosphoryl, dipropoxyphosphoryl, dibutoxyphosphoryl, dipentyloxyphosphoryl, dihexyloxyphosphoryl, etc.);

Aromatic acyl such as aroyl (e.g., benzoyl, toluoyl, naphthoyl, etc.); ar(lower)alkanoyl [e.g., phenyl(lower)alkanoyl (e.g., phenylacetyl, phenylpropanol, phenylbutanoyl, phenylisobutanoyl, phenylpentanoyl, phenylhexanoyl, etc.), naphthtyl(lower)alkanoyl (e.g., naphthylacetyl, naphthylpropanoyl, naphthylbutanoyl, naphthylisobutanoyl, etc.); ar(lower)alkenoyl [e.g., phenyl(lower)alkenoyl (e.g., phenylpropenoyl, phenylpropenoyl, phenylbutenoyl, phenylmethacryloyl, phenylpentenoyl, phenylhexenoyl, etc.), naphthyl(lower)alkenoyl (e.g., naphthylpropenoyl, naphthylbutenoyl, etc.); ar(lower)alkoxycarbonyl [e.g., phenyl(lower)alkoxycarbonyl (e.g., benzyloxycarbonyl, etc.), etc.]; aryloxycarbonyl (e.g., phenoxycarbonyl, naphthyloxycarbonyl, etc.); aryloxy(lower)alkanoyl (e.g., phenoxyacetyl, phenoxypropionyl, etc.); arylcarbamoyl (e.g., phenylcarbamoyl, etc.); arylthiocarbamoyl (e.g., phenylthiocarbamoyl, etc.); arylglyoxyloyl (e.g., phenylglyoxyloyl, naphthylglyoxyloyl, etc.); arylsulfonyl (e.g., phenylsulfonyl, p-tolylsulfonyl, etc.); or the like.

Heterocyclic acyl such as heterocyclic carbonyl; heterocyclic(lower)alkanoyl (e.g., heterocyclicacetyl, heterocyclicpropanoyl, heterocyclicbutanoyl, heterocyclicpentanoyl, heterocyclichexanoyl, etc.); heterocyclic(lower)alkenoyl (e.g., heterocyclicpropenoyl, heterocyclicbutenoyl, heterocyclicpentenoyl, heterocyclichexenoyl, etc.); heterocyclicglyoxyloyl; or the like; and the like.

Amino protective groups in the context of the invention are the customary amino protective groups used in peptide chemistry. These include benzyloxycarbonyl, 2,4-dimethoxybenzyloxycarbonyl, 4-methoxybenzyloxycarbonyl, methoxycarbonyl, ethoxycarbonyl, tert-butoxycarbonyl, allyloxycarbonyl, phthaloyl, 2,2,2-trichloroethoxycarbonyl, fluorenyl-9-methoxycarbonyl, formyl, acetyl, 2-chloroacetyl, 2,2,2-trifluoroacetyl, 2,2,2-trichloroacetyl, benzoyl, 4-chlorobenzoyl, 4-bromobenzoyl, 4-nitrobenzoyl, phthalimido, isovaleroyl or benzyloxymethylene, 4-nitrobenzyl, 2,4-dinitobenzyl, 4-nitrophenyl, 4-methoxyphenyl, triphenylmethyl, etc.

Suitable salts of the object aminoalcohol derivatives [I] are pharmaceutically acceptable salts and include conventional non-toxic salts such as an inorganic acid addition salt [e.g. hydrochloride, hydrobromide, sulfate, phosphate, etc.], an organic acid addition salt [e.g. formate, acetate, trifluoroacetate, oxalate, maleate, fumarate, tartrate, methanesulfonate, benzenesulfonate, toluenesulfonate, etc.], an alkali metal salt [e.g. sodium salt, potassium salt, etc.] or the like.

Preferred embodiments of the object compound [I] are as follows:
X₁ is bond or —OCH₂—;
X₂ is (CH₂)ₙ— in which n is 1;
X₃ is —O—;
R₁ is phenyl which may be substituted with one or two substituent(s) selected from the group consisting of halogen, nitro, amino, benzyloxy, benzyloxycarbonylamino, hydroxy and loweralkylsulfonylamino; or pyridyl which may have amino.
R₂ is hydrogen
R₃ is hydroxy(lower)alkyl; and
R₄ is pyridyl which may be substituted with carbamoyl, lower alkoxycarbonyl, carboxy, cyano, nitro, amino, hydroxy(lower)alkyl, mono(or di)(lower)-alkylcarbamoyl, lower alkyl, halogen, lower alkylsulfonylamino, phenylsulfonylamino or lower alkanoyl; phenyl which may be substituted with halogen; quinolyl which may be substituted with lower alkoxycarbonyl, nitro, carbamoyl, carboxy, halogen or lower alkoxy; naphthyl; benzothiazolyl; pyridyl N-oxide; pyrimidinyl; naphthyridinyl; pyrazinyl; imidazo[1,2-a]pyridyl; quinoxalinyl which may be substituted with halogen; acridinyl which may be substituted with halogen and lower alkoxy; or isoquinolyl which may be substituted with halogen;

More preferred embodiment of the object compound [I] are as follows:
X₁ is bond or —OCH₂—;
X₂ is —(CH₂)ₙ in which n is 1;
X₃ is —O—;
R¹ is phenyl which may be substituted with one or two substituent(s) selected from the group consisting of halogen, nitro, amino, benzyloxy, benzyloxycarbonylamino, hydroxy and lower alkylsulfonylamino;
R₂ is hydrogen;
R₃ is hydroxy(lower)alkyl; and
R₄ is pyridyl which may be substituted with carbamoyl, lower alkoxycarbonyl, carboxy, cyano, nitro, amino, hydroxy(lower)alkyl, mono(or di)(lower)alkylcarbamoyl, lower alkyl, halogen, lower alkylsulfonylamino, phenylsulfonylamino or, lower alkanoyl; phenyl which may be substituted with halogen; quinolyl which may be substituted with lower alkoxycarbonyl, nitro, carbamoyl, carboxy, halogen or lower alkoxy; naphthyl; benzothiazolyl; pyridyl N-oxide; pyrimidinyl; naphthyridinyl; pyrazinyl; imidazo[1,2-a]pyridyl; quinoxalinyl which may be substituted with halogen; acridinyl which may be substituted with halogen and lower alkoxy; or isoquinolyl which may be substituted with halogen;

More preferred embodiment of the object compound [I) are as follows:
X₁ is bond or —OCH₂—;
X₂ is —(CH₂)ₙ in which n is 1;
X₃ is —O—;
R₁ is pyridyl which may have amino;
R₂ is hydrogen;
R₃ is hydroxy(lower)alkyl; and
R₄ is pyridyl which may have hydroxy(lower)alkyl.

The processes for preparing the object compound [I] are explained in detail in the following.

Process 1

The object compound [I] or a salt thereof can be prepared by reacting a compound [II] with a compound [III] or a salt thereof.

Suitable salt of the compound [III] may be the same as those exemplified for the compound [I].

The reaction is preferably carried out in the presence of a base such as an alkali metal carbonate [e.g. sodium carbonate, potassium carbonate, etc.], an alkaline earth metal carbonate [e.g. magnesium carbonate, calcium carbonate, etc.], an alkali metal bicarbonate [e.g. sodium bicarbonate, potassium bicarbonate, etc.], tri(lower)alkylamine [e.g. trimethylamine, triethylamine, etc.], picoline or the like.

The reaction is usually carried out in a conventional solvent, such as an alcohol [e.g. methanol, ethanol, propanol, isopropanol, etc.], diethyl ether, tetrahydrofuran, dioxane, or any other organic solvent which does not adversely influence the reaction.

The reaction temperature is not critical, and the reaction can be carried out under cooling to heating.

Process 2

The object compound [Ib] or a salt thereof can be prepared by subjecting a compound [Ia] or a salt thereof to elimination reaction of the amino protective group.

Suitable salts of the compounds [Ia] and [Ib] may be the same as those exemplified for the compound [I].

This reaction can be carried out in the manner disclosed in Example 8 or Example 30, or similar manners thereto.

Process 3

The object compound [Ic] or a salt thereof can be prepared by reacting a compound [III] or a salt thereof with a compound [IV].

Suitable salts of the compound [III] may be the same as those exemplified for the compound [I].

The reaction can be carried out in the presence of the base such as an alkali metal carbonate [e.g., sodium carbonate, potassium carbonate, etc.], an alkaline earth metal carbonate [e.g., magnesium carbonate, calcium carbonate, etc.], an alkali metal bicarbonate [e.g., sodium bicarbonate, potassium bicarbonate, etc.], tri(lower)alkylamine [e.g., trimethylamine, triethylamine, etc.], picoline or the like.

The reaction is usually carried out in a conventional solvent, such as an alcohol [e.g., methanol, ethanol, propanol, isopropanol, etc.], diethyl ether, tetrahydrofuran, dioxane, or any other organic solvent which does not have adverse effect on the reaction.

The reaction temperature is not critical, and the reaction can be carried out under cooling to heating.

The reaction can also be carried out in the manner disclosed in Example 72 or similar manner thereof.

Process 4

The object compound [Id] or a salt thereof can be prepared by reacting a compound [V] or a salt thereof with a compound [VI].

Suitable salts of the compound [V] may be the same as those exemplified for the compound [I].

The reaction can also be carried out in the manner disclosed in Example 78 or similar manners thereto.

The compounds obtained by the above processes can be isolated and purified by a conventional method such as pulverization, recrystallization, column chromatography, reprecipitation, or the like, and converted to the desired salt in conventional manners, if necessary.

It is to be noted that the compound [I] and the other compounds may include one or more stereoisomers due to asymmetric carbon atoms, and all of such isomers and mixture thereof are included within the scope of this invention.

It is further to be noted that isomerization or rearrangement of the object compound [I] may occur due to the effect of the light acid, base or the like, and the compound obtained as the result of said isomerization or rearrangement is also included within the scope of the present invention.

It is also to be noted that the solvating form of the compound [I] (e.g. hydrate, etc.) and any form of the crystal of the compound [I] are included within the scope of the present invention.

The object compound [I] or a salt thereof possesses gut selective sympathomimetic, anti-ulcerous, anti-pancreatitis, lipolytic and anti-pollakiuria activities, and are useful for the treatment and/or prevention of gastrointestinal disorders caused by smooth muscle contractions in human beings or animals, and more particularly to methods for the treatment and/or prevention of spasm or hyperanakinesia in case of irritable bowel syndrome, gastritis gastric ulcer, duodenal ulcer, enteritis, cholecystopathy, cholangitis, urinary calculus and the like; for the treatment and/or prevention of ulcer such as gastric ulcer, duodenal ulcer, peptic ulcer, ulcer causes by non steroidal anti-inflammatory drugs, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis or the like; and for the treatment and/or prevention of pancreatitis, obesity, diabetes, glycosuria, hyperlipidemia, hypertension, atherosclerosis, glaucoma, melancholia, depression, and the like, and the treatment and/or prevention of a wasing condition, weight loss, emerciation or the like.

The object compound (I) or a pharmaceutically acceptable salt thereof can be usually administered to mammals including human being in the form of a conventional pharmaceutical composition such as capsule, micro-capsule, tablet, granule, powder, troche, syrup, aerosol, inhalation, solution, injection, suspension, emulsion, suppository-or the like.

The effective ingredient may usually be administered with a unit dose of 0.01 mg/kg to 50 mg/kg, one to four times a day. However, the above dosage may be increased or decreased according to age, weight, conditions of patients or methods of administration.

In order to show the usefulness of the ethanolamine derivative in the present invention for the prophylactic and therapeutic treatment of above-mentioned diseases in a human being or an animal, the pharmacological test data of the representative compound thereof is shown in the following.

Test 1

Effect on the Increase in Intravesical Pressure Induced by Carbachol in Anesthetized Dog

Test Compound

2(S)-2-[(2S)-2-hydroxy-3-(phenoxy)propylamino]-3-[4-(7-methoxyquinolin-4-yloxy)phenyl]propan-1-ol

(This compound was obtained according to Example 78.)

Test Method

Female Beagle dogs weighing 8.0-15.0 kg were fasted for 24 hours and maintained under halothane anesthesia. A 12F Foley catheter was lubricated with water soluble jelly, inserted into the urethral orifice and advanced approximately 10 cm until the balloon tip was placed well inside the bladder. The balloon was then inflated with 5 ml of room air and catheter slowly withdrawn just part the first resistance that is felt at the bladder neck. Urine was completely drained out thought the catheter, and 30 ml of biological saline was infused. The catheter was connected to pressure transducer, and intravesical pressure was continuously recorded. The test compound was injected by intra-duodenal route at 30 minutes before the administration of carbachol (1.8 μg/kg).

Test Results

| Treatment | Increase in intravesical pressure (mmHg) |
|---|---|
| Control | 9.3 |
| Test Compound | 5.5 |
| (0.32 mg/kg) | |

(N = 2)

The following Preparations and Examples are given for the purpose of illustrating this invention.

Preparation 1

Under nitrogen, a mixture of 2-chloronicotinic acid (1.6 g), methyl iodide (0.69 ml) and potassium carbonate (1.5 g) in N,N-dimethylformamide (20 ml) was stirred at room temperature for 4 hours. To the mixture was added ethyl acetate, and insoluble materials were filtered off. The filtrate was evaporated in vacuo. The residue was dissolved in ethyl acetate, washed successively with water and brine, dried over sodium sulfate, and evaporated in vacuo to give 2-chloronicotinic acid methyl ester (1.7 g).

NMR (CDCl₃, δ): 3.97 (3H, s), 7.34 (1H, dd, J=4.8, 7.7 Hz), 8.18 (1H, dd, J=2.0, 7.7 Hz), 8.53 (1H, dd, J=2.0, 4.8 Hz)

Preparation 2

Under nitrogen, to a solution of 2-chloronicotinic acid methyl ester (1.7 g) in toluene (9.9 ml) was added diisopropylaluminum hydride (0.94M in hexane, 23 ml) at −78° C., and the mixture was stirred for 5 minutes. To the mixture were added aqueous 1M Rochelle salt and ethyl acetate, and the mixture was vigorously stirred in a warm water bath for 30 minutes. The organic layer was separated. The aqueous layer was extracted with ethyl acetate 3 times. The combined organic layers were washed successively with aqueous 1M Rochelle salt and brine, dried over sodium sulfate, and evaporated in vacuo to give (2-chloropyridin-3-yl)methanol (1.3 g).

NMR (CDCl₃, δ): 2.57 (1H, br s), 4.80 (2H, s), 7.29 (1H, dd, J=4.8, 7.6 Hz), 7.91 (1H, dd, J=1.9, 7.6 Hz), 8.31 (AH, dd, J=1.9, 4.8 Hz)

Preparation 3

The following compound was obtained according to a similar manner to that of Preparation 2.

(6-Chloropyridin-3-yl)methanol

NMR (CDCl₃, δ): 4.72 (2H, s), 7.32 (1H, d, J=8.2 Hz), 7.70 (1H, dd, J=2.4, 8.2 Hz), 8.32 (1H, d, J=2.3 Hz)

Preparation 4

Under nitrogen, a mixture of (2-chloropyridin-3-yl)methanol (1.3 g) and manganese (IV) oxide (6.3 g) in dichloromethane (13 ml) was stirred at room temperature for 5 days. The mixture was diluted with dichloromethane. Therein was added silica gel and the mixture was stirred for 30 minutes. After filtration, the filtrate was evaporated in vacuo to give 2-chloropyridine-3-carbaldehyde (1.0 g).

NMR (CHCl₃, δ): 7.43 (1H, ddd, J=0.8, 4.8, 7.7 Hz), 8.25 (1H, dd, J=2.1, 7.7 Hz), 8.62 (1H, dd, J=2.0, 4.7 Hz), 10.46 (1H, d, J=0.8 Hz)

Preparation 5

Under nitrogen, a mixture of (S)-[1-hydroxymethyl-2-(4-hydroxyphenyl)ethyl]carbamic acid tert-butyl ester (J. Med. Chem. 1992, 35, 1259-1266) (1.3 g), 2-chloropyridine-3-carbaldehyde (1.0 g) and potassium carbonate (0.97 g) in N,N-dimethylformamide (13 ml) was stirred at 60° C. for 72 hours. The mixture was diluted with ethyl acetate, and insoluble materials were filtered off. The filtrate was evaporated in vacuo. The residue was dissolved in ethyl acetate, washed successively with aqueous saturated sodium bicarbonate and brine, dried over sodium sulfate, and evaporated in vacuo. The residue was purified by column chromatography on silica gel (dichloromethane:methanol=50:1) to give (S)-{2-[4-(3-formylpyridin-2-yloxy)phenyl]-1-hydroxymethylethyl}carbamic acid tert-butyl ester (1.2 g).

NMR (CDCl₃, δ): 1.44 (9H, s), 2.36 (1H, br s), 2.88 (2H, d, J=7.1 Hz), 3.5-3.8 (2H, m), 3.89 (1H, br s), 4.7-4.9 (1H, m), 7.1-7.2 (3H, m), 7.30 (2H, d, J=80.5 Hz), 8.25 (1H, dd, J=2.0, 7.5 Hz), 8.34 (1H, dd, J=2.1, 4.9 Hz), 10.55 (1H, s)

Preparation 6

The following compounds were obtained according to a similar manner to that of Preparation 5.

(1) (S)-{2-[4-(3-Cyanopyridin-2-yloxy)phenyl]-1-(hydroxymethyl)ethyl]carbamic acid tert-butyl ester

NMR (CDCl₃, δ): 1.43 (9H, s), 2.88 (2H, d, J=7.1 Hz), 3.5-4.0 (3H, m), 7.05-7.15 (3H, m), 7.25-7.35 (2H, m), 8.01 (1H, dd, J=2.0, 7.6 Hz), 8.31 (1H, dd, J=2.0, 5.0 Hz).

(2) (S)-{2-[4-(5-Formylpyridin-2-yloxy)phenyl]-1-(hydroxymethyl)ethyl}carbamic acid tert-butyl ester

NMR (CDCl₃, δ): 1.43 (9H, s), 2.88 (2H, d, J=7.1 Hz), 3.5-4.0 (3H, m), 7.01-7.13 (3H, m), 7.30 (2H, d, J=8.5 Hz), 8.19 (1H, dd, J=2.3, 8.6 Hz), 8.62 (1H, d, J=2.2 Hz), 9.98 (1H, s)

(3) (S)-[2-[4-(3,5-Dichloropyridin-4-yloxy)phenyl]-1-(hydroxymethyl)ethyl)carbamic acid tert-butyl ester

NMR (CDCl₃, δ): 1.41 (9H, s), 2.81 (2H, d, J=7.1 Hz), 3.5-3.9 (3H, m), 6.78 (2H, d, J=8.7 Hz), 7.17 (2H, d, J=8.6 Hz), 7.26 (1H, s), 8.56 (1H, s)

(4) (S)-{2-[4-(6-Fluoropyridin-2-yloxy)phenyl]-1-(hydroxymethyl)ethyl}carbamic acid tert-butyl ester

NMR (CDCl₃, δ): 1.43 (9H, s), 2.86 (2H, d, J=7.1 Hz), 3.5-4.0 (3H, m), 6.61 (1H, dd, J=2.4, 7.6 Hz), 6.71 (1H, dd, J=1.3, 7.9 Hz), 7.05-7.10 (2H, m), 7.25-7.30 (2H, m), 7.74 (1H, q, J=8.0 Hz)

Preparation 7

To a mixture of (S)-{2-[4-(3-formylpyridin-2-yloxy)-phenyl]-1-(hydroxymethyl)ethyl)carbamic acid tert-butyl ester (5.2 g), 35% hydrogen peroxide (2.5 ml) and potassium dihydrogen phosphate (7.5 g) in a mixture of acetonitrile (60 ml) and water (15 ml) was dropwise added sodium chlorite (80% purity, 4.7 g) at room temperature, and the mixture was stirred at the same temperature for 1 hour. While cooling in ice-water bath, to the mixture was added sodium sulfite (3.5 g). After removal of the bath, to this was added aqueous 1M citric acid to make it acidic, and extracted with ethyl acetate. The organic layer was washed successively with water and brine, dried over sodium sulfate, and evaporated in vacuo. The crude product was triturated with diisopropyl ether to give (S)-2-[4-(2-tert-butoxycarbonylamino-3-hydroxypropyl)phenoxy]nicotinic acid (4.8 g).

NMR (DMSO-d₆, δ): 1.34 (9H, s), 2.5-2.65 (1H, m), 2.83 (1H, dd, J=5.2, 13.8 Hz), 3.2-3.5 (2H, m), 3.5-3.7 (1H, m), 4.71 (1H, br s), 6.62 (1H, d, J=8.3 Hz), 7.01 (2H, d, J=8.5 Hz), 7.17-7.23 (3H, m), 8.2-8.3 (2H, m), 13.18 (1H, br s)

Preparation 8

The following compound was obtained according to a similar manner to that of Preparation 7.

(S)-6-[4-(2-tert-Butoxycarbonylamino-3-hydroxypropyl)-phenoxy]nicotinic acid

NMR (DMSO-d₆, δ): 1.33 (9H, s), 2.5-2.7 (1H, m), 2.86 (1H, dd, J=5.0, 13.8 Hz), 3.2-3.5 (3H, m), 6.65 (1H, d, J=8.4 Hz), 7.08 (2H, d, J=8.3 Hz), 7.26 (2H, d, J=8.5 Hz), 8.26 (1H, dd, J=2.4, 8.5 Hz), 8.66 (1H, d, J=2.1 Hz)

Preparation 9

Under nitrogen, a suspension of (S)-2-[4-(2-tert-butoxycarbonylamino-3-hydroxypropyl)phenoxy]nicotinic acid (5.7 g), methyl iodide (1.0 ml), potassium carbonate (2.4 g) in N,N-dimethylformamide (28 ml) was stirred at room temperature for 5 hours. The mixture was diluted with ethyl acetate and insoluble materials were filtered off. The filtrate was evaporated in vacuo. The residue was dissolved in ethyl acetate, washed with aqueous saturated sodium bicarbonate, dried over sodium sulfate, and evaporated in vacuo. The residue was purified by column chromatography on silica gel (hexane:ethyl acetate=1:1) to give (S)-2-[4-(2-tert-butoxycarbonylamino-3-hydroxypropyl)phenoxy]nicotinic acid methyl ester (5.7 g).

NMR (CDCl₃, δ): 1.43 (9H, s), 2.3-2.5 (1H, br s), 2.86 (2H, d, J=7.1 Hz), 3.5-4.0 (3H, m), 3.94 (3H, s), 4.76 (1H, d, J=7.5 Hz), 7.02-7.12 (3H, m), 7.23-7.27 (2H, m), 8.24-8.29 (2H, m)

Preparation 10

The following compound was obtained according to a similar manner to that of Preparation 9.

(S)-6-[4-(2-tert-Butoxycarbonylamino-3-hydroxypropyl)-phenoxy]nicotinic acid methyl ester

NMR (CDCl₃, δ): 1.43 (9H, s), 2.87 (2H, d, J=7.2 Hz), 3.5-4.0 (3H, m), 3.92 (3H, s), 6.93 (1H, d, J=9.2 Hz), 7.09 (2H, d, J=8.5 Hz), 7.28 (2H, d, J=8.5 Hz), 8.27 (1H, dd, J=2.4, 8.6 Hz), 8.82 (1H, d, J=1.8 Hz)

Preparation 11

To a solution of (S)-2-[4-(2-tert-butoxycarbonylamino-3-hydroxypropyl)phenoxy]nicotinic acid methyl ester (5.6 g) in methanol (56 ml) was added 4N hydrogen chloride in ethyl acetate (35 ml) at room temperature, and the solution was stirred at the same temperature overnight. The mixture was evaporated in vacuo, and the residue was triturated with ethyl acetate to give (S)-2-[4-(2-amino-3-hydroxypropyl)-phenoxy]nicotinic acid methyl ester dihydrochloride (5.0 g).

NMR (DMSO-d₆, δ): 2.8-3.0 (2H, m), 3.3-3.6 (3H, m), 3.85 (3H, s), 7.08 (2H, d, J=8.4 Hz), 7.23-7.33 (3H, m), 8.26-8.31 (2H, m)

Preparation 12

The following compounds were obtained according to a similar manner to that of Preparation 11.

(1) (S)-2-[4-(2-Amino-3-hydroxypropyl)phenoxy]-nicotinonitrile hydrochloride

NMR (DMSO-d₆, δ): 2.85-3.05 (2H, m), 3.3-3.7 (3H, m), 7.21 (2H, d, J=8.5 Hz), 7.29-7.38 (3H, m), 8.37-8.45 (2H, m)

(2) (S)-6-[4-(2-Amino-3-hydroxypropyl)phenoxy]nicotinic acid methyl ester dihydrochloride

NMR (DMSO-d₆, δ): 2.75-3.05 (2H, m), 3.30-3.60 (3H, m), 3.86 (3H, s), 7.11-7.20 (3H, m), 7.35 (2H, d, J=8.5 Hz), 8.32 (1H, dd, J=2.4, 8.6 Hz), 8.69 (1H, d, J=1.8 Hz)

(3) (S)-2-Amino-3-[4-(3,5-dichloropyridin-4-yloxy)phenyl]-propan-1-ol hydrochloride

NMR (DMSO-d₆, δ): 2.7-2.95 (2H, m), 3.25-3.60 (3H, m), 6.90 (2H, d, J=8.6 Hz), 7.27 (2H, d, J=8.7 Hz), 8.79 (2H, s)

(4) (S)-2-Amino-3-[4-(6-fluoropyridin-2-yloxy)phenyl]-propan-1-ol hydrochloride

NMR (DMSO-d₆, δ): 2.75-3.05 (2H, m), 3.30-3.65 (3H, m), 6.85-6.95 (2H, m), 7.15 (2H, d, J=8.5 Hz), 7.35 (2H, d, J=8.5 Hz), 8.03 (1H, q, J=8.0 Hz)

Preparation 13

Under nitrogen, a mixture of (6-chloropyridin-3-yl)methanol (1.2 g) and manganese (IV) oxide (6.0 g) in N,N-dimethylformamide (12 ml) was stirred at room temperature for 6 days. The mixture was diluted with ethyl acetate and insoluble materials were filtered off. The filtrate was evaporated in vacuo. The residue was dissolved in ethyl acetate, washed successively with aqueous saturated sodium bicarbonate and brine; dried over sodium sulfate, and evaporated in vacuo. The residue was purified by column chromatography on silica gel. (dichloromethane) to give 6-chloropyridine-3-carbaldehyde (0.58 g).

NMR (CDCl₃, δ): 7.52 (1H, d; J=8.2 Hz), 8.15 (1H, dd, J=2.4, 8.3 Hz), 8.88 (1H, d, J=2.3 Hz), 10.10 (1H, s)

Preparation 14

Under nitrogen, a mixture of (S)-[1-hydroxymethyl-2-(4-hydroxyphenyl)ethyl]carbamic acid tert-butyl ester (10 g), 2,6-dibromopyridine (18 g) and potassium carbonate (10 g) in N,N-dimethylformamide (100 ml) was stirred at 120° C. for 3 days. The mixture was diluted with ethyl acetate, and insoluble materials were filtered off. The filtrate was evaporated in vacuo. The residue was dissolved in ethyl acetate, washed successively with aqueous saturated sodium bicarbonate (twice) and brine, dried over sodium sulfate, and evaporated in vacuo. The residue was purified by column chromatography on silica gel (dichloromethane:methanol 100:1), followed by trituration with diisopropyl ether to give (S)-4-[4-(6-bromopyridin-2-yloxy)benzyl]oxazolidin-2-one (6.6 g).

NMR (DMSO-d₆, δ): 2.75-2.85 (2H, m), 3.95-4.15 (2H, m)., 4.25-4.40 (1H, m), 6.99 (1H, d, J=8.1 Hz), 7.11 (2H, d, J=8.4 Hz), 7.25-7.40 (3H, m), 7.79 (1H, t, J=7.7 Hz)

Preparation 15

The following compound was obtained according to a similar manner to that of Preparation 14.

(S)-4-[4-(6-Chloropyridin-2-yloxy)benzyl]oxazolidin-2-one

NMR (CDCl₃, 6): 2.7-3.1 (2H, m), 4.0-4.3 (2H, m), 4.4-4.6 (1H, m), 6.80 (1H, d, J=8.1 Hz), 7.0-7.3 (5H, m), 7.64 (1H, t, J=7.9 Hz)

Preparation 16

Under nitrogen, to a solution of butyllithium (1.6M in hexane, 14 ml) in tetrahydrofuran (20 ml) was added a solution of (S)-74-[4-(6-bromopyridin-2-yloxy)benzyl]-oxazolidin-2-one (3.5 g) in tetrahydrofuran (15 ml) at −78° C., and the mixture was stirred at the same temperature for 15 minutes. To it was added N,N-dimethylformamide (1.7 ml), the dry ice bath was removed to allow to come to room temperature. The mixture was poured into water and extracted with ethyl acetate. The organic layer was washed with brine, dried over sodium sulfate, and evaporated in vacuo. The residue was dissolved in ethanol, and treated with aqueous sodium hydrogen sulfite for 10 minutes. After evaporation in vacuo and partition between ethyl acetate and water, the aqueous layer was made basic with aqueous sodium carbonate and extracted with ethyl acetate twice. The organic layer was washed with brine, dried over sodium sulfate, and evaporated in vacuo. The residue was purified by column chromatography on silica gel (dichloromethane:methanol=50:1), followed by crystallization from ethyl acetate to give (S)-6-[4-(2-oxo-oxazolidin-4-ylmethyl)phenoxy]pyridine-2-carbaldehyde (0.64 g).

NMR (DMSO-d₆, δ): 2.7-2.95 (2H, m), 3.95-4.15 (2H, m), 4.25-4.40 (1H, m), 7.16 (2H, d, J=8.4 Hz), 7.25-7.40 (3H, m), 7.70 (1H, d, J=7.3 Hz), 8.09 (1H, t, J=7.5 Hz), 9.73 (1H, s)

Preparation 17

Under nitrogen, to a suspension of (S)-6-[4-(2-oxo-oxazolidin-4-ylmethyl)phenoxy]pyridine-2-carbaldehyde (0.43 g) in methanol (14 ml) was added sodium borohydride (54 mg) at 5° C., and the mixture was stirred at the same temperature for 5 minutes. The mixture was evaporated in vacuo. To the residue were added water and ethyl acetate. After separation, the organic layer was washed with brine, dried over sodium sulfate, and evaporated in vacuo to give (S)-4-[4-(6-hydroxymethylpyridin-2-yloxy)benzyl]oxazolidin-2-one (0.43 g).

NMR (DMSO-d₆, δ): 2.7-2.9 (2H, m), 3.95-4.15 (2H, m), 4.25-4.40 (1H, m), 4.39 (2H, d, J=5.8 Hz), 6.77 (1H, d, J=8.1 Hz), 7.04 (2H, d, J=8.5 Hz), 7.15-7.30 (3H, m), 7.84 (1H, t, J=7.8 Hz).

Preparation 18

The following compound was obtained according to a similar manner to that of Preparation 17.

(S)-4-[4-(5-Hydroxymethylpyridin-2-yloxy)benzyl]-oxazolidin-2-one

NMR (CDCl₃, δ): 2.8-3.0 (2H, m), 4.0-4.25 (2H, m), 4.50-4.60 (1H, m), 4.67 (2H, d, J=5.7 Hz), 6.95 (1H, d, J=8.5 Hz), 7.11 (2H, d, J=8.6 Hz), 7.22 (2H, d, J=8.6 Hz), 7.77 (1H, dd, J=2.4, 8.4 Hz), 8.13 (1H, d, J=1.9 Hz)

Preparation 19

Under nitrogen, a solution of (S)-4-[4-(6-hydroxymethylpyridin-2-yloxy)benzyl]oxazolidin-2-one (0.46 g), (R)-3-chlorostyrene oxide (0.46 g) and potassium carbonate (0.41 g) in N,N-dimethylformamide (4.6 ml) was stirred at 80° C. for 72 hours. The mixture was diluted with ethyl acetate and insoluble materials were filtered off. The filtrate was evaporated in vacuo. The residue was dissolved in ethyl acetate, washed successively with aqueous saturated sodium bicarbonate and brine, dried over sodium sulfate, and evaporated in vacuo. The residue was purified by column chromatography on silica gel (dichloromethane:methanol=50:1) to give (4S)-3-[(2R)-2-(3-chlorophenyl)-2-hydroxyethyl]-4-[4-(6-hydroxymethylpyridin-2-yloxy)benzyl]-oxazolidin-2-one (0.39 g) and (5R)-5-(3-chlorophenyl)-3-{(1S)-2-hydroxy-1-[4-(6-hydroxymethylpyridin-2-yloxy)benzyl]-ethyl}oxazolidin-2-one (76 mg).

(1) (4S)-3-[(2R)-2-(3-Chlorophenyl)-2-hydroxyethyl]-4-[4-(6-hydroxymethylpyridin-2-yloxy)benzyl]oxazolidin-2-one

NMR (DMSO-d₆, δ): 2.68 (1H, dd, J=7.9, 13.4 Hz), 3.05-3.15 (1H, m), 3.2-3.35 (1H, m), 3.47 (1H, dd, J=4.4, 14.2 Hz), 3.95-4.10 (1H, m), 4.1-4.4 (2H, m), 4.38 (2H, d, J=5.8 Hz), 4.75-4.90 (1H, m), 6.77 (1H, d, J=7.8 Hz), 7.07 (2H, d, J=8.5 Hz), 7.20-7.50 (8H, m), 7.82 (1H, t, J=7.8 Hz)

(2) (5R)-5-(3-Chlorophenyl)-3{(1S)-2-hydroxy-1-[4-(6-hydroxymethylpyridin-2-yloxy)benzyl]ethyl}oxazolidin-2-one

NMR (DMSO-d₆, δ): 2.6-2.9 (2H, m), 3.3-3.4 (1H, m), 3.5-3.6 (2H, m), 3.9-4.15 (2H, m), 4.38 (2H, d, J=5.8 Hz), 5.53 (1H, d, J=5.5, 8.8 Hz), 6.76 (1H, d, J=8.1 Hz), 6.96 (2H d, J=8.3 Hz), 7.0-7.5 (7H, m), 7.83 (1H, t, J=7.8 Hz)

Preparation 20

The following compounds were obtained according to a similar manner to that of Preparation 19.

(1) (4S)-3-[(2R)-2-(3-Chlorophenyl)-2-hydroxyethyl]-4-[4-(5-hydroxymethylpyridin-2-yloxy)benzyl]oxazolidin-2-one

NMR (CDCl₃, δ): 1.90-2.05 (1H, m), 2.55-2.75 (1H, m), 3.14 (1H, dd, J=4.0, 13.6 Hz), 3.2-3.4 (1H, m), 3.28 (1H, dd, J=8.1, 14.8 Hz), 3.67 (1H, dd, J=2.8, 14.8 Hz), 4.05-4.30 (3H, m), 4.66 (2H, d, J=5.6 Hz), 4.95-5.05 (1H, m), 6.92 (1H, d, J=8.4 Hz), 7.08 (2H, d, J=8.8 Hz), 7.15 (2H, d, J=8.8 Hz), 7.2-7.4 (3H, m), 7.41 (1H, s), 7.75 (1H, dd, J=2.4, 8.4 Hz), 8.12 (1H, d, J=2.1 Hz)

(2) (5R)-5-(3-Chlorophenyl)-3-{(1S)-2-hydroxy-1-[4-(5-hydroxymethylpyridin-2-yloxy)benzyl]ethyl}oxazolidin-2-one

NMR (DMSO-d₆, δ): 2.6-2.9 (2H, m), 3.3-3.45 (1H, m), 3.5-3.6 (2H, m), 3.9-4.15 (2H, m), 4.47 (2H, d, J=5.6 Hz), 5.5-5.6 (1H, m), 6.9-7.2 (6H, m), 7.27 (1H, s), 7.3-7.45 (2H, m), 7.79 (1H, dd, J=2.4, 8.4 Hz), 8.05-8.1 (1H, m)

Preparation 21

The following compounds were obtained according to a similar manner to that of Preparation 19.

(1) (4S)-3-[(2R)-2-(3-Chlorophenyl)-2-hydroxyethyl]-4-[4-(pyridin-2-yloxy)benzyl]oxazolidin-2-one

NMR (DMSO-d₆, δ): 2.55-2.8 (1H, m), 3.05-3.15 (1H, m), 3.2-3.6 (2H, m), 3.95-4.1 (1H, m), 4.1-4.4 (2H, m), 4.8-4.9 (1H, m), 6.9-7.5 (10H, m), 7.8-7.9 (1H, m), 8.1-8.2 (1H, m)

(2) (5R)-5-(3-Chlorophenyl)-3-{(1S)-2-hydroxy-1-[4-(pyridin-2-yloxy)benzyl]ethyl}oxazolidin-2-one

NMR (DMSO-d₆, δ): 2.65-2.9 (2H, m), 3.3-3.4 (1H, m), 3.5-3.6 (2H, m), 3.9-4.15 (2H, m), 5.5-5.6 (1H, m), 6.95-7.3 (8H, m), 7.35-7.4 (2H, m), 7.8-7.9 (1H, m), 8.15-8.2 (1H, m)

Preparation 22

A mixture of (S)-4-[4-(6-chloropyridin-2-yloxy)benzyl]-oxazolidin-2-one (4.1 g), 10% palladium on activated carbon (50% wet, 0.82 g) and N,N-diisopropylethylamine (2.3 ml) in ethanol (41 ml) was stirred at room temperature in the presence of hydrogen at an atmospheric pressure for 2 hours, and filtered. The filtrate was evaporated in vacuo. The residue was dissolved in ethyl acetate, washed successively with water and brine, dried over sodium sulfate, and evaporated in vacuo. The residue was purified by column chromatography on silica gel (dichloromethane:methanol=25:1) to give (S)-4-[4-(pyridin-2-yloxy)benzyl]oxazolidin-2-one (2.8 g).

NMR (CDCl₃, δ): 2.8-2.95 (2H, m), 4.0-4.25 (2H, m), 4.45-4.55 (1H, m), 6.9-7.3 (6H, m), 7.65-7.75 (1H, m), 8.15-8.20 (1H, m)

Preparation 23

Under nitrogen, to a solution of (S)-{2-[4-(5-formylpyridin-2-yloxy)phenyl]-1-hydroxymethylethyl}carbamic acid tert-butyl ester (1.4 g) in dichloromethane (37 ml) was added thionyl chloride (0.30 ml) at 5° C., and the mixture was stirred at room temperature overnight. The mixture was evaporated in vacuo, and the residue was vigorously stirred in a mixture of ethyl acetate and aqueous saturated sodium bicarbonate at 50° C. for 30 minutes. After separation, the organic layer was washed with brine, dried over sodium sulfate, and evaporated in vacuo. The residue was purified by column chromatography on silica gel (dichloromethane:ethyl acetate=3:2), followed by trituration with ethyl acetate to give (S)-6-[4-(2-oxo-oxazolidin-4-ylmethyl)phenoxy]pyridine-3-carbaldehyde (0.52 g).

NMR (CDCl₃, δ): 2.80-3.05 (2H, m), 4.05-4.25 (2H, m), 4.45-4.60 (1H, m), 7.05-7.35 (5H, m), 8.21 (1H, d, J=2.3, 8.6 Hz), 8.62 (1H, d, J=2.2 Hz), 9.99 (1H, s)

Preparation 24

Under nitrogen, to a solution of m-fluorophenol (1.74 ml) and sodium hydride (772 mg) in dimethylformamide (25 ml) was added (2S)-(+)-glycidyl nosylate (5.0 g) at 0° C. and the mixtue was stirred at the same temperature for 0.5 hour. The mixture was allowed to warm to room temperature and stirred for 2.5 hours at this temperature. The resulting mixture was poured into 10% aqueous ammonium chloride solution, and extracted with ethyl acetate. The organic layer was washed with brine, dried over magnesium sulfate, and evaporated in vacuo. The residue was chromatographed (hexane-ethyl acetate) over silica gel to afford (2S)-3-(3-fluorophenoxy)-1,2-epoxypropane (2.82 g) as a colorless powder.

NMR (CDCl₃, δ): 2.75 (1H, dd, J=3.0, 4.8 Hz) 2.90 (1H, t, J=4.8 Hz), 3.35 (1H, m), 3.90 (1H, dd, J=5.7, 11 Hz), 4.20 (1H, dd, J=3.0, 11 Hz), 6.50-6.70 (3H, m), 7.20-7.25 (1H, m)

MS (m/z): 169 (M+1)

Preparation 25

The following compounds were synthesized according to a similar manner to that of Preparation 24.

(1) (2S)-3-(4-Chlorophenoxy)-1,2-epoxypropane (2.79 g) as a Colorless Powder

NMR (DMSO-d₆, δ): 2.70 (1H, dd, J=2.6, 6 Hz), 2.85 (1H, t, J=4.3 Hz), 3.28-3.36 (1H, m), 3.80 (1H, dd, J=6, 11 Hz), 4.30 (1H, dd, J=2.6, 11 Hz), 6.70-6.80 (2H, m), 7.30-7.40 (2H, m)

(2) (2S)-3-(2-Chlorophenoxy)-1,2-epoxypropane (1.6 g) as a Colorless Powder

NMR (CDCl₃, δ): 2.80-3.00 (2H, m), 3.35-3.40 (1H, m), 4.05 (1H, dd, J=5.2, 11 Hz), 4.30 (1H, dd, J=3.1, 11 Hz), 6.80-7.00 (2H, m), 7.10-7.2.0 (1H, m), 7.30-7.40 (1H, m)

Preparation 26

Under nitrogen, a mixture of (S)-2-[4-(2-tert-butoxycarbonylamino-3-hydroxy-propyl)phenoxy]nicotinic acid (1.0 g), ethyl iodide (0.2 ml), potassium carbonate (425 mg) and N,N-dimethylformamide (10 ml) was stirred at room temperature for 3 hours. The mixture was diluted with ethyl acetate and insoluble materials were filtered off. The filtrate was evaporated in vacuo. The residue was dissolved in ethyl acetate, washed with aqueous saturated sodium bicarbonate, dried over sodium sulfate, and evaporated in vacuo. The residue was purified by column chromatography on silica gel (hexane:ethyl acetate=1:1) to give (S)-2-[4-(2-tert-butoxycarbonylamino-3-hydroxypropyl)phenoxy]nicotinic acid ethyl ester (1.01 g) as a colorless form.

MALDI-MS (m/z): 439 (M+Na)

Preparation 27

The following compound was synthesized according to a similar manner to that of Preparation 32.

(S)-2-[4-(2-Amino-3-hydroxypropyl)phenoxy]nicotinic acid ethyl ester hydrochloride (5.06 g) as a Colorless Powder

MS (m/z): 317 (M+1)

Preparation 28

Thionyl chloride (30.2 ml) was added dropwise to a solution of (R)-2-amino-3-(4-hydroxyphenyl)propionic acid hydrochloride (25.0 g) in methanol(250 ml) under ice water cooling over 10 minutes and the mixture was stirred at room temperature for 3 hours. The mixture was evaporated in vacuo and the residue was triturated with diisopropyl ether to give (R)-2-amino-3-(4-hydroxyphenyl)propionic acid methyl ester hydrochloride (33.9 g).

NMR (DMSO-d₆, δ): 2.90-3.10 (2H, m), 3.66 (3H, s), 4.17 (1H, t, J=6.1 Hz), 6.70 (2H, d, J=9 Hz), 7.02 (2H, d, J=9 Hz), 9.47 (1H, br s)

Preparation 29

A solution of (S)-2-amino-3-(4-hydroxyphenyl)propionic acid methyl ester hydrochloride (33.9 g), di-tert-butyl dicarbonate (30.5 g) and triethylamine (50.9 ml) in tetrahydrofuran (500 ml) was stirred at room temperature for 3 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate and evaporated in vacuo to give (S)-2-tert-butoxycarbonylamino-3-(4-hydroxyphenyl)propionic acid methyl ester (42.28 g) as a colorless powder.

NMR (DMSO-d₆, δ): 1.30 (9H, s), 2.70-2.80 (2H, m), 3.58 (3H, s), 4.10-4.20 (1H, m), 6.60 (2H, d, J=8.3 Hz), 7.20 (2H, d, J=8.3 Hz), 9.21 (1H, br s)

Preparation 30

Under nitrogen, to a solution of (R)-2-tert-butoxycarbonylamino-3-(4-hydroxyphenyl)propionic acid methyl ester (42.28 g) in tetrahydrofuran (400 ml) was added lithium borohydride (7.16 g) at 5° C., and the mixture was stirred at the same temperature for 5 hours. The mixture was evaporated in vacuo. To the residue was added water and extracted with ethyl acetate. The organic layer was washed with brine, dried over sodium sulfate, and evaporated in vacuo. The residue was triturated with diisopropyl ether to give (R)-(1-(4-hydroxybenzyl)-2-hydroxyethyl]carbamic acid tert-butyl ester (4.63 g) as a colorless powder.

NMR (DMSO-d₆, δ): 1.32 (9H, s), 2.30-2.70 (2H, m), 3.20-3.50 (3H, m), 6.60 (2H, d, J=8.3 Hz), 7.00 (2H, d, J=8.3 Hz), 9.10 (1H, br s)

MS (m/z): 290 (M+1)

Preparation 31

Under nitrogen, a mixture of (R)-[1-hydroxymethyl-2-(4-hydroxyphenyl)ethyl]carbamic acid tert-butyl ester (9.75 g), 2-chloro-3-cyanopyridine (5.06 g) and potassium carbonate (6.04 g) in N,N-dimethylformamide (100 ml) was stirred at 60° C. for 72 hours. The mixture was diluted with ethyl acetate, and insoluble materials were filtered off. The filtrate was evaporated in vacuo. The residue was dissolved in ethyl acetate, washed with aqueous saturated sodium bicarbonate and brine, dried over sodium sulfate, and evaporated in vacuo. The residue was triturated with diisopropyl ether to give (R)-{2-[4-(3-cyanopyridin-2-yloxy)phenyl]-1-hydroxymethylethyl}carbamic acid tert-butyl ester (9.13 g) as a colorless powder.

MALDI-MS (m/z): 392 (M+Na)

Preparation 32

To a solution of (R)-{2-[4-(3-cyanopyridin-2-yloxy)-phenyl]-1-hydroxymethylethyl]carbamic acid tert-butyl ester (9.13 g) in dioxane (20 ml) was added 4N hydrogen chloride in dioxane (10 ml) at room temperature, and the solution was stirred at the same temperature overnight. The mixture was evaporated in vacuo, and the residue was triturated with ethyl acetate to give (2R)-2-amino-3-[4-(3-cyanopyridin-2-yloxy)phenyl]propanol dihydrochloride (7.69 g).

MS (m/z): 392 (M+1)

Preparation 33

Thionyl chloride (32.2 ml) was added dropwise to a solution of (R,S)-2-amino-3-(3-hydroxyphenyl)propionic acid hydrochloride (20.0 g) in methanol (200 ml) under ice water cooling over 10 minutes and the mixture was stirred at room temperature for 3 hours. The mixture was evaporated in vacuo and the residue was triturated with diisopropyl ether to give (R,S)-2-amino-3-(3-hydroxyphenyl)propionic acid methyl ester hydrochloride (25.57 g).

NMR (DMSO-d₆, δ): 2.90-3.15 (2H, m), 3.68 (3H, s), 4.18 (1H, t, J=6 Hz), 6.60-6.70 (3H, m), 7.10 (1H, t, J=8 Hz), 8.60-8.70 (1H, br s), 9.50-9.60 (1H, br s)

MS (m/z): 196 (M+1)

Preparation 34

A solution of (R,S)-2-amino-3-(3-hydroxyphenyl)propionic acid methyl ester hydrochloride (25.57 g), di-tert-butyl dicarbonate (21.15 g) and triethylamine (50.9 ml) in dioxane (500 ml) was stirred at room temperature for 3 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate and evaporated in vacuo to give (R,S)-2-tert-butoxycarbonylamino-3-(3-hydroxyphenyl)propionic acid methyl ester (28.14 g) as a colorless powder.

NMR (DMSO-d₆, δ): 1.33 (9H, s), 2.70-2.95 (2H, m), 3.60 (3H, s), 3.97-4.17 (1H, m), 6.60-6.70 (3H, m), 7.05-7.33 (1H, m), 9.28 (1H, br s)

Preparation 35

Under nitrogen, to a solution of (R,S)-2-tert-butoxycarbonylamino-3-(4-hydroxyphenyl)propionic acid methyl ester (28.14 g) in tetrahydrofuran (300 ml) was added lithium borohydride (5.19 g) at 5° C., and the mixture was stirred at the same temperature for 5 hours. The mixture was evaporated in vacuo. To the residue was added water and extracted with ethyl acetate. The organic layer was washed with brine, dried over sodium sulfate, and evaporated in vacuo. The residue was triturated with diisopropyl ether to give (R,S)-[1-(4-hydroxybenzyl)-2-hydroxyethyl]carbamic acid tert-butyl ester (26.73 g) as a colorless powder.

NMR (CD₃Cl, δ): 1.41 (9H, s), 2.70-2.80 (2H, d, J=7 Hz), 3.50-3.80 (3H, m), 6.60-6.75 (3H, m), 7.05-7.23 (1H, m)

Preparation 36

The following compound was synthesized according to a similar manner to that of Preparation 31.

(R,S)-{2-[3-(3-Cyanopyridin-2-yloxy)phenyl]-1-hydroxymethylethyl}carbamic acid tert-butyl ester as a Colorless Form

NMR (DMSO-d₆, δ): 2.50-2.60 (1H, m), 2.80-3.00 (1H, m), 3.30-3.40 (1H, m), 3.50-3.60 (1H, m), 4.60-4.70 (1H, m), 7.00-7.15 (3H, m), 7.30-7.40 (2H, m), 8.30-8.40 (2H, m)

Preparation 37

Under nitrogen, a mixture of (S)-[1-hydroxymethyl-2-(4-hydroxyphenyl)ethyl]carbamic acid tert-butyl ester (5.0 g), 2-chloro-3-nitropyridine (1.52 g), potassium carbonate (1.56 g) and N,N-dimethylformamide (50 ml) was stirred at 60° C. for 72 hours. The mixture was diluted with ethyl acetate, and insoluble materials were filtered off. The filtrate was evaporated in vacuo. The residue was dissolved in ethyl acetate, washed with aqueous saturated sodium bicarbonate and brine, dried over sodium sulfate, and evaporated in vacuo. The residue was triturated with diisopropyl ether to give (S)-{2-[4-(3-nitropyridin-2-yloxy)phenyl]-1-hydroxymethyl-ethyl}carbamic acid tert-butyl ester (4.11 g) as a yellow form.

MS (m/z): 389 (M+1)

Preparation 38

To a solution of (1S)-(1-hydroxymethyl-2-[4-(3-nitropyridin-2-yloxy)phenyl]ethyl)carbamic acid tert-butyl ester (4.11 g) in methanol (20 ml) was added 4N hydrogen chloride in dioxane (20 ml) at room temperature, and the solution was stirred at the same temperature overnight. The mixture was evaporated in vacuo, and the residue was triturated with diisopropyl ether to give (2S)-2-amino-3-[4-(3-nitropyridin-2-yloxy)phenyl]propanol hydrochloride (3.2 g) as a yellow powder.

MS (m/z): 290 (M+1)

Preparation 39

Under nitrogen, a solution of (2S)-2-((2S)-2-hydroxy-3-phenoxypropylamino)-3-[4-(3-nitropyridin-2-yloxy)phenyl]-propanol (1.18 g) and di-tert-butyl dicarbonate (0.52 g) in N,N-dimethylformamide (10 ml) was stirred at room temperature for 9 hours. The mixture was diluted with ethyl acetate and poured into water. The organic layer was washed with aqueous 10% potassium hydrogensulfate and brine, dried over sodium sulfate and evaporated in vacuo. The residue was purified by column chromatography on silica gel (hexane:ethyl acetate=3:2) to give {(1S)-1-hydroxymethyl-2-[4-(3-nitropyridin-2-yloxy)phenyl]ethyl}-(2S)-(2-hydroxy-3-phenoxypropyl)carbamic acid tert-butyl ester (1.34 g).

MALDI-MS (m/z): 562 (M+Na)

Preparation 40

Under nitrogen, to a suspension of (S)-{2-[4-(3-formylpyridin-2-yloxy)phenyl]-1-hydroxymethylethyl}carbamic acid tert-butyl ester (11.32 g) in methanol (100 ml) was added sodium borohydride (1.15 g) at 5° C., and the mixture was stirred at the same temperature for 1 hour. The mixture was evaporated in vacuo. To the residue were added water and ethyl acetate. After separation, the organic layer was washed with brine, dried over sodium sulfate, and evaporated in vacuo to give (S)-{2-[4-(3-hydroxymethylpyridin-2-yloxy)-phenyl]-1-hydroxymethylethyl}carbamic acid tert-butyl ester (9.88 g) as a colorless powder.

MS (m/z): 375 (M+1)

Preparation 41

The following compounds were synthesized according to a similar manner to that of Preparation 32.

(1) (2S)-2-Amino-3-[4-(3-hydroxymethylpyridin-2-yloxy)-phenyl]propanol hydrochloride (6.88 g) as a colorless powder

MS (m/z): 275 (M+1)

(2) 2-[4-(2-Amino-3-hydroxypropyl)phenoxy]nicotinamide dihydrochloride (13.37 g) as a Colorless Powder

NMR (CD₃OD, δ): 2.80-3.10 (2H, m), 3.50-3.80 (3H, m), 7.10-7.42 (5H, m), 8.10-8.20 (1H, m), 8.30-8.40 (1H, m)

MS (m/z): 288 (M+1)

Preparation 42

A solution of (S)-}2-[4-(3-cyanopyridin-2-yloxy)phenyl]-1-hydroxymethylethyl}carbamic acid tert-butyl ester in methyl sulfoxide (540 ml) was added dropwise 30% hydrogen peroxide (54 ml) under ice cooling, and the solution was stirred at the same temperature for 30 minutes. The mixture was added 5N sodium hydroxide. (54 ml) and stirred at room temperature for 1 hour. The resulting mixture was acidified with hydrochloric acid to pH 3 and partitioned between water and ethyl acetate. The organic layer was washed with brine, dried over sodium sulfate, and evaporated in vacuo. The residue was triturated with diisopropyl ether to give (S)-2-[4-(2-tert-butoxycarbonylamino-3-hydroxypropyl)phenoxy]-nicotinamide (17.99 g) as a colorless powder.

NMR (CDCl₃, δ): 1.50 (9H, s), 2.90 (2H, d, J=7 Hz), 3.50-3.90 (3H, m), 7.00-7.32 (5H, m), 8.20-8.30 (1H, m), 8.50-8.60 (1H, m)

Preparation 43

To a solution of (S)-{1-hydroxymethyl-2-[4-(3-hydroxymethylpyridin-2-yloxy)phenyl]ethyl}carbamic acid tert-butyl ester (3.4 g) in a mixture of 1,4-dioxane (4 ml) and tetrahydrofuran (6 ml) was added 4N hydrogen chloride in 1,4-dioxane (10 ml) at room temperature, and the mixture was stirred at room temperature for 1 hour. After evaporation in vacuo, the residue was triturated with hexane and dried in vacuo to give. (S)-2-amino-3-[4-(3-hydroxymethylpyridin-2-yloxy)phenyl]propan-1-ol dihydrochloride (4.1 g).

NMR (DMSO-d₆, δ): 2.6-3.05 (2H, m), 3.1-3.8 (3H, m), 4.62 (2H, s), 7.05 (2H, d, J=8.5 Hz), 7.15 (1H, ABq, J=4.9, 7.3 Hz), 7.30 (2H, d, J=8.5 Hz), 7.8-8.1 (2H, m)

Preparation 44

Under nitrogen, powdered potassium hydroxide (74 g) was added to dimethylsulfoxide (5 ml) at room temperature, and the mixture was stirred at the same temperature for 1.5 hours. To this one were added (S)-[1-hydroxymethyl-2-(4-hydroxyphenyl)ethyl]carbamic acid tert-butyl ester (300 mg) and 2-chloropyrimidine (129 mg), and the mixture was stirred at room temperature for 24 hours. The resulting mixture was poured into saturated aqueous sodium hydrogencarbonate and the aqueous mixture was extracted with ethyl acetate. The organic layer was washed with brine, dried over anhydrous magnesium sulfate, and evaporated in vacuo. The residue was purified by column chromatography on silica gel (hexane:ethyl acetate=1:1 to 1:2) to give (S)-{1-hydroxymethyl-2-[4-(pyrimidin-2-yloxy)phenyl]ethyl carbamic acid tert-butyl ester (300 mg).

NMR (CDCl₃, δ): 1.42 (9H, s), 2.87 (2H, d, J=7.1 Hz), 3.5-4.0 (3H, m), 7.04 (1H, t, J=4.8 Hz), 7.13 (2H, d, J=80.5 Hz), 7.29 (2H, d, J=8.5 Hz), 8.56 (2H, d, J=4.8 Hz)

Preparation 45

The following compounds were obtained according to a similar manner to that of Preparation 43.

(1) (S)-2-[4-(2-Amino-3-hydroxypropyl)phenoxy]nicotinic acid ethyl ester dihydrochloride (460 mg)

NMR (DMSO-d₆, δ): 1.3 (3H, t, J=7.1 Hz), 2.75-3.15 (2H, m), 3.25-3.85 (2H, m), 4.1-4.5 (3H, m), 6.77 (1H, d, J=8.4 Hz), 7.05-7.4 (4H, m), 8.2-8.4 (2H, m)

(2) (S)-2-Amino-3-[4-(pyrimidin-2-yloxy)phenyl]propan-1-ol dihydrochloride (230 mg)

NMR (DMSO-d₆, δ): 2.75-3.05 (2H, m), 3.25-3.65 (3H, m), 7.16 (2H, d, J=8.5 Hz), 7.27 (1H, t, J=4.8 Hz), 7.34 (2H, d, J=8.5 Hz), 8.65 (2H, d, J=4.8 Hz)

(3) (S)-2-Amino-3-[4-(pyrazin-2-yloxy)phenyl]propan-1-ol dihydrochloride (190 mg)

NMR (DMSO-d₆, δ): 2.75-3.1 (2H, m), 3.2-3.7 (3H, m), 7.17 (2H, d, J=8.5 Hz), 7.36 (2H, d, J=8.5 Hz), 8.1-8.3 (1H, m), 8.38 (1H, d, J=2.7 Hz), 8.53 (1H, m)

Preparation 46

The following compound was obtained according to a similar manner to that of Preparation 44.

(S)-{1-Hydroxymethyl-2-[4-(pyrazin-2-yloxy)phenyl]ethyl}carbamic acid tert-butyl ester (190 mg)

NMR (CDCl₃, δ): 1.43 (9H, s), 3.5-3.75 (2H, m), 3.8-3.95 (1H, m), 7.05-7.15 (2H, m), 7.2-7.35 (2H, m), 8.08-8.10 (1H, m), 8.26 (1H, d, J=2.7 Hz), 8.14 (1H, m)

Preparation 47

Under nitrogen, to a solution of (S)-[1-hydroxymethyl-2-(4-hydroxyphenyl)ethyl]carbamic acid tert-butyl ester (24 g) in dichloromethane (500 ml) were added 2,2-dimethoxypropane (34 ml) and p-toluenesulfonic acid monohydrate (1.7 g) at room temperature, and the mixture was stirred at the same temperature for 60 hours. The resulting mixture was poured into saturated aqueous sodium hydrogencarbonate and the aqueous mixture was extracted with ethyl acetate. The organic layer was washed with brine, dried over anhydrous magnesium sulfate, and evaporated in vacuo to get a solid. To the solid was added hexane so as to triturate and then the slurry was stirred for 12 hours, followed by filtration and dryness in vacuo to give (S)-4-(4-hydroxybenzyl)-2,2-dimethyloxazolidine-3-carboxylic acid tert-butyl ester (22 g).

NMR (DMSO-d₆, δ): 1.3-1.55 (15H, m), 2.4-2.6 (1H, m), 2.8-2.95 (1H, m), 3.6-4.0 (3H, m), 6.69 (2H, d, J=8.2 Hz), 6.98 (2H, d, J=8.4 Hz)

Preparation 48

Under nitrogen, to a suspension of 2-chloroisonicotinic acid (2.0 g) in methanol (50 ml) was added a catalytic amount of concentrated sulfuric acid at room temperature, and the mixture was refluxed for 11 hours. The resulting mixture was poured into saturated aqueous sodium hydrogencarbonate and the aqueous mixture was extracted with ethyl acetate. The organic layer was washed with brine, dried over anhydrous magnesium sulfate, and evaporated in vacuo to give methyl 2-chloroisonicotinate (2.0 g).

NMR (CDCl₃, δ): 3.98 (3H, s), 7.75-7.8 (1H, m), 7.89 (1H, m), 8.55 (1H, d, J=5.1 Hz)

Preparation 49

A solution of methyl 2-chloroisonicotinate (1.9 g) and 28% ammonium hydroxide in water (4 ml) in methanol (20 ml) was sealed with stirring for 24 hours to result in the formation of precipitates. They were collected by filtration and dried in vacuo to give 2-chloroisonicotinamide (1.1 g).

NMR (DMSO-d₆, δ): 7.75-7.85 (1H, m), 7.87 (1H, s), 8.56 (1H, d, J=5.1 Hz)

Preparation 50

Under nitrogen, powdered potassium hydroxide (210 mg) was added to dimethylsulfoxide (20 ml) at room temperature, and the mixture was stirred at the same temperature for 1 hour. To this one were added (S)-4-(4-hydroxybenzyl)-2,2-dimethyloxazolidine-3-carboxylic acid tert-butyl ester (980 mg) and 2-chloroisonicotinamide (500 mg), and the mixture was stirred at 100° C. for 17 hours. The resulting mixture was poured into water and the aqueous mixture was extracted with ethyl acetate. The organic layer was washed with brine, dried over anhydrous magnesium sulfate, and evaporated in vacuo. The residue was purified by column chromatography on silica gel (chloroform:methanol=100:1 to 50:1) to give (S)-4-[4-(4-carbamoylpyridin-2-yloxy)benzyl]-2,2-dimethyloxazolidine-3-carboxylic acid tert-butyl ester (1.0 g).

NMR (CDCl₃, δ): 1.5-1.7 (15H, m), 2.65-2.75 (1H, m), 3.05-3.3 (1H, m), 3.75-3.85 (2H, m), 3.95-4.2 (1H, m), 7.0-7.15 (2H, m), 7.2-7.35 (4H, m), 8.28 (1H, d, J=5.2 Hz)

Preparation 51

To a solution of (S)-4-[4-(4-carbamoylpyridin-2-yloxy)-benzyl]-2,2-dimethyloxazolidine-3-carboxylic acid tert-butyl ester (1.0 g) in a mixture of 1,4-dioxane (2.5 ml) and methanol (2.5 ml) was added 4N hydrogen chloride in 1,4-dioxane (5 ml) at room temperature, and the mixture was stirred at room temperature for 6 hours. After evaporation in vacuo, the residue was triturated with hexane and dried in vacuo to give (S)-2-[4-(2-amino-3-hydroxypropyl)phenoxy]-isonicotinamide hydrochloride (800 mg).

NMR (DMSO-d₆, δ): 2.8-3.1 (2H, m), 3.3-3.65 (3H, m), 7.12 (2H, d, J=8.4 Hz), 7.3-7.45 (3H, m), 7.5-7.6 (1H, m), 8.27 (1H, d, J=5.2 Hz)

Preparation 52

The following compounds were obtained according to a similar manner to that of Preparation 50.

(1) (S)-4-[4-(5-Carbamoylpyridin-2-yloxy)benzyl]-2,2-dimethyloxazolidine-3-carboxylic acid tert-butyl ester (1.4 g)

NMR (CDCl₃, δ): 1.3-1.7 (15H, m), 2.65-2.8 (1H, m), 3.05-3.3 (1H, m), 3.7-4.2 (3H, m), 6.9-7.35 (5H, m), 8.18 (1H, ABq, J=2.5, 8.6 Hz), 8.60 (1H, d, J=2.4 Hz)

(2) (S)-4-[4-(6-Carbamoylpyridin-2-yloxy)benzyl]-2,2-dimethyloxazolidine-3-carboxylic acid tert-butyl ester (930 mg)

NMR (CDCl₃, δ): 1.4-1.7 (15H, m), 2.55-2.85 (1H, m), 3.1-3.3 (1H, m), 3.75-4.2 (3H, m), 7.05-7.15 (3H, m), 7.2-7.4 (2H, m), 7.8-7.95 (2H, m)

(3) (S)-4-[4-(2-Carbamoylpyridin-4-yloxy)benzyl]-2,2-dimethyloxazolidine-3-carboxylic acid tert-butyl ester (1.4 g)

NMR (CDCl₃, δ): 1.45-1.7 (15H, m), 2.65-2.8 (1H, m), 3.05-3.3 (1H, m), 3.75-4.2 (3H, m), 6.95-7.1 (3H, m), 7.2-7.4 (2H, m), 7.8-7.9 (1H, m), 8.4-8.45 (1H, m)

(4) (S)-2,2-Dimethyl-4-[4-(3-methylcarbamoylpyridin-2-yloxy)benzyl]oxazolidine-3-carboxylic acid tert-butyl ester (970 mg)

NMR (CDCl₃, δ): 1.45-1.75 (15H, m), 2.65-2.8 (1H, m), 3.0-3.3 (4H, m), 3.75-3.9 (2H, m), 3.95-4.2 (1H, m), 7.05-7.4 (5H, m), 8.20 (1H, ABq, J=2.0, 4.8 Hz), 8.64 (1H, ABq, J=1.9, 7.6 Hz)

(5) (S)-2,2-Dimethyl-4-[4-(quinolin-2-yloxy)benzyl]-oxazolidine-3-carboxylic acid tert-butyl ester (650 mg)

NMR (CDCl₃, δ): 1.5-1.7 (15H, m), 2.65-2.8 (1H, m), 3.1-3.3 (1H, m), 3.8-4.25 (3H, m), 7.08 (1H, d, J=8.8 Hz), 7.15-7.5 (5H, m), 7.55-7.7 (1H, m), 7.75-7.85 (2H, m), 8.12 (1H, d, J=8.8 Hz)

Preparation 53

The following compound was obtained according to a similar manner to that of Preparation 48.

The obtained crude methyl 6-bromopicolinate was used successively in next step.

Preparation 54

A solution of methyl 6-bromopicolinate obtained the previous step, and 28% ammonium hydroxide in water (6 ml) in methanol (40 ml) was sealed with stirring for 12 hours. The resulting mixture was evaporated in vacuo and dried to give 6-bromopicolinamide (2.8 g).

(+) APCI-MS (m/z): 201, 203 (M+1)⁺

Preparation 55

The following compounds were obtained according to a similar manner to that of Preparation 51.

(1) (S)-6-[4-(2-Amino-3-hydroxypropyl)phenoxy]nicotinamide hydrochloride (1.1 g)

NMR (DMSO-d₆, δ): 2.8-3.05 (2H, m), 3.3-3.7 (3H, m), 7.05-7.2 (2H, d, J=8.5 Hz), 8.28 (1H, ABq, J=2.5, 8.6 Hz), 8.63 (1H, d, J=2.4 Hz)

(2) (S)-6-[4-(2-Amino-3-hydroxypropyl)phenoxy]pyridine-2-carboxylic acid amide hydrochloride (690 mg)

NMR (DMSO-d₆, δ): 2.8-3.1 (2H, m), 3.3-3.7 (3H, m), 7.1-7.55 (5H, m), 7.7-7.9 (1H, m), 8.0-8.15 (1H, m)

(3) (S)-4-[4-(2-Amino-3-hydroxypropyl)phenoxy]pyridine-2-carboxylic acid amide hydrochloride (1.1 g)

NMR (DMSO-d₆, δ): 2.85-3.15 (2H, m), 3.3-3.8 (4H, m), 7.25 (2H, d, J=8.4 Hz), 7.36 (1H, ABq, J=2.6, 6.0 Hz), 7.48 (2H, d, J=8.4 Hz), 7.87 (1H, d, J=2.3 Hz), 8.64 (1H, d, J=6.2 Hz)

(4) (S)-2-[4-(2-Amino-3-hydroxypropyl)phenoxy]-N-methylnicotinamide hydrochloride (730 mg)

NMR (DMSO-d₆, δ): 2.7-3.0 (5H, m), 3.3-3.7 (3H, m), 7.05-7.4 (5H, m), 8.1-8.2 (2H, m)

(5) (S)-2-Amino-3-(4-phenoxyphenyl)propan-1-ol hydrochloride (730 mg)

NMR (DMSO-d₆, δ): 2.75-3.0 (2H, m), 3.25-3.7 (3H, m), 6.9-7.1 (4H, m), 7.14 (1H, t, J=7.2 Hz), 7.25-7.5 (4H, m)

(6) (S)-2-Amino-3-[4-(4-chlorophenoxy)phenyl]propan-1-ol hydrochloride (980 mg)

NMR (DMSO-d₆, δ): 2.75-3.05 (2H, m), 3.2-3.75 (3H, m), 6.95-7.1 (4H, m), 7.25-7.5 (4H, m)

(7) (S)-2-Amino-3-[4-(naphthalen-1-yloxy)phenyl]propan-1-ol hydrochloride (630 mg)

NMR (DMSO-d₆, δ): 2.75-3.0 (2H, m), 3.2-3.7 (3H, m), 6.9-7.1 (3H, m), 7.30 (2H, d, J=8.5 Hz), 7.45-7.65 (3H, m), 7.74 (1H, d, J=8.2 Hz), 7.9-8.3 (2H, m)

(8) (S)-2-Amino-3-[4-(quinolin-2-yloxy)phenyl]propan-1-ol dihydrochloride (550 mg)

NMR (DMSO-d₆, δ): 2.8-3.1 (2H, m), 3.3-3.7 (3H, m), 7.2-7.55 (6H, m), 7.6-7.7 (1H, m), 7.97 (1H, d, J=7.9 Hz), 8.43 (1H, d, J=8.8 Hz)

(9) (S)-2-Amino-3-[4-(quinolin-3-yloxy)phenyl]propan-1-ol dihydrochloride (1.3 g)

NMR (DMSO-d₆, δ): 2.8-3.15 (2H, m), 3.25-3.7 (3H, m), 7.18 (2H, d, J=8.4 Hz), 7.40 (2H, d, J=8.5 Hz), 7.65-8.5 (5H, m), 8.9-9.0 (1H, m)

(10) (S)-2-Amino-3-(4-phenylsulfanylphenyl)propan-1-ol hydrochloride (560 mg)

NMR (DMSO-d₆, δ): 2.75-3.0 (2H, m), 3.25-3.6 (3H, m), 7.25-7.5 (9H, m)

Preparation 56

Under nitrogen, a mixture of picolinic acid (5 g) and thionyl chloride (12.5 ml) was refluxed for 180 hours. The reaction mixture was diluted with chloroform, and cooled in an ice bath. To this one was added methanol (30 ml) dropwise and the mixture was stirred at the same temperature for 1 hour, followed by evaporation in vacuo. The residue was dissolved in a mixture of saturated aqueous sodium hydrogencarbonate and ethyl acetate. After separation, the organic layer was washed with brine, dried over anhydrous magnesium sulfate, and evaporated in vacuo. The residue was purified by column chromatography on silica gel (toluene:chloroform=5:1 to 1:5) to give methyl 4-chloropicolinate (3.3 g).

NMR (CDCl₃, δ): 4.03 (3H, s), 7.52 (1H, ABq, J=2.0, 5.2 Hz), 8.15 (1H, d, J=2.0 Hz), 8.66 (1H, d, J=5.2 Hz)

Preparation 57

The following compound was obtained according to a similar manner to that of Preparation 49.

4-Chloropicolinamide (2.4 g)

NMR (DMSO-d₆, δ): 7.77 (1H, ABq, J=2.1, 5.3 Hz), 8.04 (1H, d, J=2.1 Hz), 8.63 (1H, d, J=5.3 Hz)

Preparation 58

Under nitrogen, a solution of 2-chloronicotinic acid (4.0 g) in N,N-dimethylformamide (40 ml) were added methylamine hydrochloride (1.9 g), 1-(3-dimethylaminopropyl)-3-ethylcarbodiimide (5.1 ml) and 1-hydroxybenzotriazole (3.8 g) at 5° C., and the mixture was stirred at room temperature for 12 hours. The resulting mixture was poured into 1N aqueous sodium hydroxide and the aqueous mixture was extracted with ethyl acetate 7 times. The organic layer was dried over anhydrous magnesium sulfate and evaporated in vacuo. The residue was purified by column chromatograpy on silica gel (chloroform:methanol=50:1) to give N-methyl-2-chloronicotinamide (2.9 g).

NMR (CDCl₃, δ): 2.95-3.05 (3H, m), 7.32 (1H, ABq, J=7.7 Hz), 8.00 (1H, ABq, J=2.0, 7.6 Hz), 8.41 (1H, ABq, J=2.0, 4.8 Hz)

Preparation 59

To a mixture of (S)-4-(4-hydroxybenzyl)-2,2-dimethyloxazolidine-3-carboxylic acid tert-butyl ester (1.0 g), phenylboronic acid (790 mg), copper(II) acetate (590 mg) and powdered molecular sieves 4A (1.0 g) were added dichloromethane (33 ml) and pyridine (1.3 ml) at room temperature, and the mixture was stirred at the same temperature for 48 hours. After removal of insoluble materials by filtration, the filtrate was poured into 0.1N hydrochloric acid and the aqueous mixture was extracted with ethyl acetate. The organic layer was washed successively with saturated aqueous sodium hydrogencarbonate and brine, dried over anhydrous magnesium sulfate, and evaporated in vacuo. The residue was purified by column chromatography on silica gel (hexane:chloroform=1:1 to only chloroform) to give (S)-2,2-dimethyl-4-(4-phenoxybenzyl)oxazolidine-3-carboxylic acid tert-butyl ester (1.0 g).

NMR (CDCl₃, δ): 1.4-1.75 (15H, m), 2.6-2.75 (1H, m), 3.0-3.3 (1H, m), 3.7-4.2 (4H, m), 6.9-7.4 (9H, m)

Preparation 60

The following compound was obtained according to a similar manner to that of Preparation 59.

(1) (S)-4-[4-(4-Chlorophenoxy)benzyl]-2,2-dimethyloxazolidine-3-carboxylic acid tert-butyl ester (1.4 g).

NMR (CDCl₃, δ): 1.45-1.7 (15H, m), 2.6-2.75 (1H, m), 3.05-3.25 (1H, m), 3.7-4.2 (3H, m) 6.9-7.0 (4H, m), 7.1-7.3 (4H, m)

(2) (S)-2,2-Dimethyl-4-[4-(naphthalen-1-yloxy)benzyl]-oxazolidine-3-carboxylic acid tert-butyl ester (870 mg)

NMR (CDCl₃, δ): 1.45-1.7 (15H, m), 2.6-2.75 (1H, m), 3.0-3.25 (1H, m), 3.75-4.2 (3H, m), 6.85-7.05 (3H, m), 7.1-7.65 (6H, m), 7.85-7.9 (1H, m), 8.15-8.25 (1H, m)

(3) (S)-2,2-Dimethyl-4-[4-(quinolin-3-yloxy)benzyl]-oxazolidine-3-carboxylic acid tert-butyl ester (1.6 g)

NMR (CDCl₃, δ): 1.45-1.7 (15H, m), 2.65-2.8 (1H, m), 3.05-3.3 (1H, m), 3.75-4.2 (3H, m), 7.04 (2H, d J=7.9 Hz), 7.15-7.35 (2H, m), 7.5-7.8 (4H, m), 8.1-8.2 (1H, m), 8.80 (1H, d, J=2.8 Hz)

Preparation 61

Under nitrogen, to a solution of 3-bromoquinoline (3.3 ml) and triisopropyl borate (7.8 ml) in tetrahydrofuran (50 ml) was added n-butyl lithium (1.52M in hexane, 22 ml) dropwise in acetone-dry ice bath, and the mixture was stirred at the same temperature for 1 hour and then allowed to warm to room temperature by removal of the bath. The mixture was poured into 2N hydrochloric acid and adjusted to pH 5 with 5N aqueous sodium hydroxide. After separation, the organic layer was washed with brine, dried over magnesium sulfate, and evaporated in vacuo. The residue was triturated with hexane and dried in vacuo to give 3-quinolinylboronic acid (4.4 g).

NMR (CDCl₃, δ): 7.55-7.7 (1H, m), 7.75-7.85 (1H, m), 8.00 (2H, t, J=7.3 Hz), 8.73 (1H, s), 9.19 (1H, d, J=1.6 Hz)

Preparation 62

To a solution of 4-chloroquinoline-7-carboxylic acid (2.6 g) was added potassium hydroxide (870 mg) at room temperature, and the mixture was stirred at the same temperature for 12 hours. The mixture was evaporated and dried in vacuo. Under nitrogen, to a solution of the potassium salts in N,N-dimethylformamide (60 ml) was added iodoethane (1.0 ml) at room temperature, and the mixture was stirred at 80° C. for 1.5 hours. The mixture was poured into ice-cold water with stirring to generate a precipitate. After stirred for 20 minutes, the precipitate was collected by filtration and immediately the filter cake was dissolved in ethyl acetate. The solution was dried over anhydrous magnesium sulfate and evaporated in vacuo. The residue was purified by column chromatography on silica gel (toluene:ethyl acetate=20:1 to 5:1) to give ethyl 4-chloroquinoline-7-carboxylate (2.3 g).

NMR (CDCl₃, δ): 1.46 (3H, t, J=7.1 Hz), 4.48 (2H, q, J=7.1 Hz), 7.59 (1H, d, J=4.7 Hz), 8.2-8.35 (2H, m), 8.85-8.9 (2H, m)

Preparation 63

A mixture of ethyl 4-chloroquinoline-7-carboxylate (470 mg) and 2M ammonium hydroxide in methanol (30 ml) was sealed with stirring at 100° C. for 60 hours. The reaction mixture was evaporated and dried in vacuo to give 4-chloroquinoline-7-carboxylic acid amide (420 mg).

NMR (DMSO-d₆, δ): 7.86 (1H, d, J=4.7 Hz), 8.15-8.845 (2H, m), 8.65 (1H, d, J=1.3 Hz), 8.94 (1H, d, J=4.7 Hz)

Preparation 64

Under nitrogen, to a solution of (S)-4-(4-hydroxybenzyl)-2,2-dimethyloxazolidine-3-carboxylic acid tert-butyl ester (10 g) in dichloromethane (100 ml) were added 2,6-lutidine (4.2 ml) and trifluoromethanesulfonic anhydride (6.0 ml) at 5° C., and the mixture was stirred at the same temperature for 80 minutes. The reaction mixture was poured into ice-cold 0.1N hydrochloric acid and the aqueous mixture was extracted with ethyl acetate. The organic layer was washed successively with saturated aqueous sodium hydrogencarbonate, water and brine, dried over anhydrous magnesium sulfate, and evaporated in vacuo. The residue was purified by column chromatography on silica gel (hexane:ethyl acetate=10:1) to give trifluoromethanesulfonic acid (S)-4-(3-benzyl-2,2-dimethyloxazolidin-4-ylmethyl)phenyl ester (13 g).

NMR (CDCl₃, δ): 1.35-1.7 (15H, m), 2.65-2.85 (1H, m), 3.05-3.3 (1H, m), 3.7-4.2 (3H, m), 7.15-7.4 (4H, m)

Preparation 65

Under nitrogen, to a solution of benzenethiol (0.94 ml) in tetrahydrofuran (30 ml) was added dropwise n-butyl lithium (1.52M in hexane, 6.0 ml) in acetone-dry ice bath, and the mixture was stirred at the same temperature for 20 minutes. Under nitrogen, to a solution of trifluoromethanesulfonic acid (S)-4-(3-benzyl-2,2-dimethyloxazolidin-4-ylmethyl)phenyl ester (3.6 g), lithium chloride (770 mg) and tetrakis(triphenylphosphine)palladium(0) (1.9 g) in tetrahydrofuran (40 ml) was added the above prepared solution at room temperature, and the mixture was refluxed for 40 minutes. The mixture was poured into water and the aqueous mixture was extracted with ethyl acetate. The organic layer was washed with brine, dried over anhydrous magnesium sulfate, and evaporated in vacuo. The residue was purified by column chromatography on silica gel (hexane:ethyl acetate=20:1 to 10:1) to give (S)-2,2-dimethyl-4-(4-phenylsulfanyl-benzyl)oxazolidine-3-carboxylic acid tert-butyl ester (1.8 g).

NMR (CDCl₃, δ): 1.4-1.7 (15H, m), 2.55-2.75 (1H, m), 3.0-3.25 (1H, m), 3.7-4.2 (3H, m), 7.1-7.4 (9H, m)

Preparation 66

Under nitrogen, to a solution of (S)-4-(2-amino-3-hydroxypropyl)phenol hydrochloride (5.0 g) in methanol (50 ml) was added 28% sodium methoxide in methanol (4.7 ml) at 5° C., and the mixture was stirred at the same temperature for 10 minutes. After removal of insoluble materials by filtration, the filtrate was evaporated and dried in vacuo. A mixture of the residue and benzaldehyde (2.5 ml) in toluene (50 ml) in the presence of a catalytic amount of p-toluenesulfonic acid monohydrate was refluxed for 2 hours to remove water as the toluene azeotrope, and then the mixture was evaporated in vacuo. To a solution of the residue in methanol (50 ml) was added sodium borohydrite (930 mg) under nitrogen at 5° C., and the mixture was stirred at the same temperature for 1 hour. The reaction mixture was poured into ice-cold water with stirring. After 20 minutes, ethyl acetate and brine were added, followed by separation. The organic layer was washed with brine, dried over anhydrous magnesium sulfate and evaporated in vacuo. The residue was purified by column chromatography on silica gel (chloroform:methanol=20:1 to 10:1) to give (S)-4-(2-benzylamino-3-hydroxypropyl)phenol (6.3 g).

NMR (DMSO-d₆, δ): 2.45-2.75 (3H, m), 3.15-3.45 (2H, m), 3.73 (2H, s), 6.6-6.7 (2H, m), 6.9-7.0 (2H, m), 7.15-7.35 (5H, m)

Preparation 67

The following compounds were obtained according to a similar manner to that of Example 57.

(1) 4-((2S)-2-{Benzyl[(2R)-2-(3-chlorophenyl)-2-hydroxyethyl]amino}-3-hydroxypropyl)phenol (4.3 g)

NMR (CDCl₃, δ): 2.4-2.95 (4H, m), 3.0-3.2 (1H, m), 3.45-3.9 (4H, m), 4.3-4.45 (1H, m), 6.66 (2H, d, J=8.4 Hz), 6.85 (2H, d, J=8.4 Hz), 6.95-7.4 (9H, m)

(2) 4-((2S)-2-{Benzyl[(2R)-2-(4-benzyloxy-3-nitrophenyl)-2-hydroxyethyl]amino}-3-hydroxypropyl)phenol (1.4 g)

NMR (CDCl₃, δ): 2.5-2.95 (4H, m), 3.1-3.25 (1H, m), 3.5-3.9 (4H, m), 4.3-4.4 (1H, m), 5.16 (2H, s), 6.71 (2H, d, J=8.4 Hz), 6.9-7.0 (3H, m), 7.1-7.5 (11H, m), 7.62 (1H, d, J=2.1 Hz).

Preparation 68

Potassium hydroxide powder (85% purity, 236 mg, 3.58 mmol) was added to 10 ml of dimethyl sulfoxide and the mixture was stirred at room temperature for 20 minutes. To the resulting solution was added (S)-4-(4-hydroxybenzyl)-2,2-dimethyloxazolidine-3-carboxylic acid tert-butyl ester (1.00 g, 3.25 mmol) and the whole was stirred for additional 10 minutes. Then a solution of 4-chloroquinoline (585 mg, 3.58 mmol) in dimethyl sulfoxide (1 ml) was added and the whole was heated to 100° C., stirred for 5 hours. After cooling to room temperature, the mixture was quenched by the addition of water (20 ml) and extracted with ethyl acetate (20 ml×1). The extract was washed with water (20 ml×1) and brine (20 ml×1), dried (magnesium sulfate), and then evaporated to give a yellow solid (1.38 g). The crude solid was chromatographed on a 40 g of silica gel (eluent: hexane/ethyl acetate=4/1 to 2/1) to give (S)-2,2-dimethyl-4-[4-(quinolin-4-yloxy)benzyl]oxazolidin-3-carboxylic acid tert-butyl ester (1.29 g, 92%) as a white solid.

NMR (CDCl₃, δ): 1.49-1.65 (15H, m), 2.70-2.81 (1H, m), 3.11-3.19 (1H, m), 3.78-3.91 (2H, m), 3.99-4.13 (1H, m), 6.55 (1H, d, J=5.2 Hz), 7.11-7.15 (2H, m), 7.54-7.62 (1H, m), 7.73-7.80 (1H, m), 8.10 (1H, d, J=8.4 Hz), 8.36 (1H, d, J=7.2 Hz), 8.67 (1H, d, J=5.2 Hz)

MS: 435 (M+1)

Preparation 69

To a solution of (S)-2,2-dimethyl-4-[4-(quinolin-4-yloxy)benzyl]oxazolidin-3-carboxylic acid tert-butyl ester (679 mg, 1.56 mmol) in dioxane (6.0 ml) was added 4N hydrogen chloride in dioxane (6.0 ml, 24 mmol) at room temperature and the mixture was stirred at the same temperature for 90 minutes. The solvent was removed by evaporation to give (S)-2-amino-3-[4-(quinolin-4-yloxy)phenyl]propan-1-ol dihydrochloride (920 mg, 161 mmol) as a pale yellow solid.

MS: 295 (M−HCl—Cl⁻)

Preparation 70

Potassium hydroxide powder (85% purity, 236 mg, 3.58 mmol) was added to 10 ml of dimethyl sulfoxide and the mixture was stirred at room temperature for 1 hour. To the resulting solution was added (S)-4-(4-hydroxybenzyl)-2,2-dimethyloxazolidine-3-carboxylic acid tert-butyl ester (1.00 g, 3.25 mmol) and the whole was stirred for additional 10 minutes. A solution of 5-chloro-imidazo[1,2-α]pyridine (546 mg, 3.58 mmol) in dimethyl sulfoxide (1 ml) was added and the whole was warmed to 100° C. then stirred at the same temperature for 2 hours. After cooling to room temperature, the mixture was quenched by the addition of water (20 ml) and extracted with ethyl acetate (20 ml×1). The extract was washed with water (20 ml×2) brine (20 ml×1), dried (MgSO₄), and evaporated to give a crude oil (1.32 g). The crude oil was chromatographed on a 26 g of silica gel (eluent: hexane/ethyl acetate=1/1) to give 4-[4-(imidazo[1,2-α]pyridine-5-yloxy)benzyl]-2,2-dimethyloxazolidine-3-carboxylic acid tert-butyl ester (1.05 g, 76%) as a yellow paste.

MS: 446 (M+1)

Preparation 71

To a solution of 4-[4-(imidazo[1,2-α]pyridine-5-yloxy)-benzyl]-2,2-dimethyl-oxazolidine-3-carboxylic acid tert-butyl ester (994 mg, 2.35 mmol) in dioxane (10 ml) was added 4N hydrogen chloride in dioxane (10 ml) at room temperature and the solution was stirred at the same temperature for 1 hour. The solvent was removed under reduced pressure to give a pale yellow solid. The solid was dissolved in saturated aqueous solution of sodium hydrogencarbonate (15 ml) was extracted with dichloromethane (15 ml×5). The extracts were combined and dried (magnesium sulfate), then evaporated to give (S)-2-amino-3-[4-(imidazo[1,2-α]pyridine-5-yloxy)phenyl]propan-1-ol (589 mg, 88%) as a pale yellow paste.

MS: 284 (M+1)

Preparation 72

To a solution of (S)-2-amino-3-[4-(imidazo[1,2-a]-pyridin-5-yloxy)phenyl]propan-1-ol (208 mg, 0.734 mmol) in 1,3-dimethyl-2-imidazolidinone (2.0 ml) were successively added (S)-N-[2-benzyloxy-5-[2-iodo-1-(triethylsilyloxy)-ethyl]phenyl]methanesulfonamide (495 mg, 0.881 mmol) and diisopropylethylamine (192 μl, 1.10 mmol). The solution was warmed to 100° C. and stirred for 5 hours. After cooling to room temperature, 10 ml of water was added and the mixture was extracted with ethyl acetate (100 ml×1). The extract was washed with water (10 ml×2), brine (10 ml×1), dried (magnesium sulfate), and evaporated to give a crude oil (545 mg). The oil was chromatographed on a 50 g of silica gel (eluent: chloroform/methanol=95/5) to give N-[2-benzyloxy-5-[(1R)-2-[(1S)-2-hydroxy-1-[4-(imidazo[1,2-a]pyridin-5-yloxy)benzyl]ethylamino]-1-(triethylsilyloxy)ethyl]phenyl}-methanesulfonamide (160 mg, 30%) as a colorless oil.

NMR (CDCl₃, δ): 0.53 (6H, q, J=7.7 Hz), 0.88 (9H, t, J=7.7 Hz), 1.65 (2H, br s), 2.69-2.85 (5H, m), 2.89 (3H, s), 3.21-3.27 (1H, m), 3.53 (1H, dd, J=3.5, 10.7 Hz), 4.74 (1H, t, J=5.0 Hz), 5.08 (2H, s), 5.96 (1H, d, J=7.4 Hz), 6.78 (1H, br s), 6.89-7.78 (16H, m, ArH)

MS: 717 (M+1)

Preparation 73

Potassium hydroxide powder (85% purity, 236 mg, 3.58 mmol) was added to 10 ml of dimethyl sulfoxide and the mixture was stirred at the room temperature for 45 minutes. To the mixture was added (S)-4-(4-hydroxybenzyl)-2,2-dimethyloxazolidine-3-carboxylic acid tert-butyl ester (1.00 g, 3.25 mmol) and the whole was stirred for additional 5 minutes. Further, a solution of 2-chloro-N,N-dimethylnicotinamide (546 mg, 3.58 mmol) in dimethyl sulfoxide (1 ml) was added and the whole was warmed to 100° C. The mixture was stirred at the same temperature for 3 hours then at 120° C. for 8 hours. After cooling to room temperature, the reaction mixture was quenched by the addition of water (20 ml) and extracted with ethyl acetate (20 ml×1). The extract was washed with water (20 ml×2), brine (20 ml×1), dried (magnesium sulfate), then evaporated to give a pale yellow foam (1.34 g). The crude product was chromatographed on a 40 g of silica gel (eluent: hexane/ethyl acetate=2/1 to 1/1, then 1/2) to give (S)-4-[4-[3-(dimethyl carbamoyl)pyridin-2-yloxy]benzyl]-2,2-dimethyloxazolidine-3-carboxylic acid tert-butyl ester (878 mg, 59%) as a white foam.

IR (KBr): 1695, 1643, 1419 1390 cm⁻¹

NMR (CDCl₃, δ): 1.48-1.57 (15H, m), 2.61-2.72 (1H, m), 3.03 (3H, s), 3.06-3.29 (1H, m), 3.14 (3H, s), 3.79 (2H, d, J=3.1 Hz), 3.89-4.18 (1H, m), 7.05-7.09 (3H, m), 7.21-7.27 (2H, m), 7.75 (1H, dd, J=1.9, 7.3 Hz), 8.17 (1H, dd, J=1.9, 5.0 Hz)

MS: 478 (M+Na⁺)

Preparation 74

To a solution of (S)-4-[4-[3-(dimethylcarbamoyl)pyridin-2-yloxy]benzyl]-2,2-dimethyloxazolidine-3-carboxylic acid tert-butyl ester (866 mg, 1.90 mmol) in a mixed solvent of dioxane (4.0 ml) and methanol (4.0 ml) was added 4N hydrogen chloride in dioxane (8.0 ml) at room temperature. After stirring for 4 hours, the solvent was removed by evaporation to give (S)-2-[4-(2-amino-3-hydroxypropyl)phenoxy]-N,N-dimethylnicotinamide hydrochloride (809 mg, 110%) as a pale yellow solid.

NMR (DMSO-d₆, δ): 2.77-2.89 (2H, m), 2.92 (3H, s), 3.01 (3H, s), 3.23-3.70 (3H, m), 4.77 (1H, br), 7.09 (2H, d, J=8.4 Hz), 7.21 (1H, dd, J=4.9, 7.3 Hz), 7.31 (2H, d, J=8.4 Hz), 7.83 (1H, dd, J=1.8, 7.3 Hz), 8.16 (1H, dd, J=1.8, 4.8 Hz), 8.17 (3H, br)

MS: 316 (M−Cl⁻)

Preparation 75

To a suspension of (S)-tyrosine methyl ester hydrochloride (20.0 g, 86.3 mmol) in acetonitrile (200 ml) was added dropwise triethylamine (48.1 ml, 345 mmol) at room temperature. After the addition, to the mixture was added dropwise benzyl bromide (30.8 ml, 259 mmol) over 10 minutes at room temperature. The mixture was warmed to 60° C. and stirred for 20 hours. Then, additional benzyl bromide (10.3 ml, 86.6 mmol) was added and the mixture was stirred at 60° C. for 12 hours. After cooling to room temperature, the mixture was quenched by the addition of water (400 ml) and extracted with ethyl acetate (400 ml). The extract was washed with water (400 ml×2), brine (400 ml×1), dried (magnesium sulfate), and evaporated to give an orange oil (21.3 g). The crude oil was chromatographed on a 500 g of silica gel (eluent: hexane/ethyl acetate=9/1 to 7/1) to give (S)-2-dibenzylamino-3-(4-hydroxyphenyl)propionic acid methyl ester (15.9 g, 49.1%) as a colorless oil.

NMR (CDCl₃, δ): 2.91 (1H, dd, J=8.2, 14.0 Hz), 3.05 (1H, dd, J=7.3, 14.0 Hz), 3.53 (2H, d, J=14.0 Hz), 3.63 (1H, d, J=7.7 Hz), 3.72 (3H, s, OMe), 3.95 (2H, d, J=14.0 Hz), 4.86 (1H, br s), 6.69 (2H, d, J=8.5 Hz), 6.87 (2H, d, J=8.5 Hz), 7.21-7.31 (10H, m, ArH)

MS: 376 (M+1)

Preparation 76

To a solution of (S)-2-dibenzylamino-3-(4-hydroxyphenyl)propionic acid methyl ester (15.9 g, 42.3 mmol) in acetonitrile (160 ml) was added portionwise potassium carbonate (23.4 g, 169 mmol) at room temperature. To the mixture was added chloromethyl methyl ether (12.9 ml, 170 mmol) and the whole was stirred at room temperature for 5 days. The mixture was quenched by the addition of water (160 ml) and the organic solvent was removed by evaporation. The residue was extracted with ethyl acetate (160 ml×1) and washed with water (160 ml×2), brine (160 ml×1), dried (magnesium sulfate), and evaporated to give (S)-2-dibenzylamino-3-[4-(methoxymethoxy)phenyl]propionic acid methyl ester (17.0 g, 96%) as a colorless oil.

IR (Neat): 2951, 1730, 1512, 1232, 1155, 1009 cm⁻¹

NMR (CDCl₃, δ): 2.92 (1H, dd, J=8.0, 14.0 Hz), 3.07 (1H, dd, J=7.4, 14.0 Hz), 3.41 (3H, s), 3.52 (2H, d, J=14.0 Hz), 3.63 (1H, d, J=7.7 Hz), 3.73 (3H, s), 3.94 (2H, d, J=14.0 Hz), 5.17 (2H, s), 6.91 (4H, s), 7.21-7.29 (10H, m)

MS: 420 (M+1)

Preparation 77

To a solution of (S)-2-dibenzylamino-3-[4-(methoxymethoxy)phenyl]propionic acid methyl ester (591 mg, 1.41 mmol) in tetrahydrofuran (6.0 ml) were successively added sodium borohydride (53.3 mg, 1.41 mmol) and lithium iodide (189 mg, 1.41 mmol) at room temperature. After stirring for 1 hour, the reaction mixture was warmed to 60° C. and stirred for 3 hours. Furthermore, additional sodium borohydride (53.3 mg, 1.41 mmol) and lithium iodide (189 mg, 1.41 mmol) were added and the mixture was refluxed for 9 hours. After cooling to room temperature, the mixture was quenched by the addition of aqueous saturated ammonium chloride solution (20 ml). The mixture was extracted with ethyl acetate (20 ml×2), brine (20 ml×1), dried (magnesium sulfate), and evaporated to give a colorless oil (568 mg). The crude oil was chromatographed on a 50 g of silica gel (eluent: hexane/ethyl acetate=9/1 to 7/1) to give (S)-2-dibenzylamino-3-[4-(methoxymethoxy)phenyl]propan-1-ol (366 mg, 66%) as a white solid.

IR (KBr): 3423 (br), 2925, 1510, 1234, 1153, 1074, 1005 cm⁻¹

NMR (CDCl₃, δ):2.32-2.44 (1H, m), 2.90-3.11 (3H, m), 3.35-3.55 (2H, m), 3.47 (3H, s), 3.48 (2H, d, J=13.3 Hz), 3.93 (2H, d, J=13.3 Hz), 5.15 (2H, s), 6.94 (2H, d, J=8.8 Hz), 7.01 (2H, d, J=8.8 Hz), 7.21-7.37 (10H, m)

MS: 392 (M+1)

Preparation 78

To a solution of (S)-2-dibenzylamino-3-[4-(methoxymethoxy)phenyl]propan-1-ol (319 mg, 0.815 mmol) in dimethylformamide (3.0 ml) was added sodium hydride (60% dispersion in oil, 48.9 mg, 1.22 mmol) at room temperature and the mixture was stirred at the same temperature for 80 minutes. To the mixture was added methyl iodide (76 μl, 1.22 mmol) and the mixture was stirred at room temperature for 24 hours. The mixture was quenched by the addition of water (10 ml) and extracted with ethyl acetate (10 ml×1). The extract was washed with brine (10 ml×1), dried (magnesium sulfate), and evaporated to give a pale yellow oil (360 mg). The crude oil was chromatographed on a 30 g of silica gel (eluent: hexane/ethyl acetate=4/1) to give (S)-dibenzyl-[2-[4-(methoxymethoxy)phenyl]-1-(methoxymethyl)ethyl)amine (257 mg, 78%) as a colorless oil.

IR (Neat): 2925, 1612, 1510, 1495, 1454, 1232, 1176 cm⁻¹

NMR (CDCl₃, δ): 2.73 (1H, dd, J=7.8, 13.6 Hz), 2.84 (1H, dd, J=6.7, 13.6 Hz), 3.00-3.0.5 (1H, m), 3.28 (3H, s), 3.38-3.56 (2H, m), 3.49 (3H, s, OMe), 3.75 (3H, s, OMe), 5.17 (2H, s), 6.90 (2H, d, J=8.8 Hz), 6.97 (2H, d, J=8.8 Hz), 7.17-7.25 (10H, m)

MS: 406 (M+1)

Preparation 79

A mixture of (S)-dibenzyl[2-[4-(methoxymethoxy)phenyl]-1-(methoxymethyl)ethyl]amine (214 mg, 0.510 mmol), palladium (10% on activated carbon, 50% wet, 100 mg) and methanol (2.0 ml) was hydrogenated (1 atm) for 2 hours. The catalyst was removed by filtration using Celite and washed with methanol. The filtrate was concentrated in vacuo to give (S)-2-[4-(methoxymethoxy)phenyl]-1-(methoxymethyl)ethylamine (113 mg, 98%) as a colorless oil.

NMR (CDCl₃, δ): 1.88 (2H, br s), 2.54 (1H, dd, J=7.5, 13.5 Hz), 2.74 (1H, dd, J=4.9, 13.5 Hz), 3.21-3.40 (3H, m), 3.37 (3H, s, OMe), 3.48 (3H, s, OMe), 5.16 (2H, s), 6.98 (2H, d, J=8.6 Hz), 7.12 (2H, d, J=8.6 Hz)

MS: 226 (M+1)

Preparation 80

To a solution of (S)-2-dibenzylamino-3-[4-(methoxymethoxy)phenyl]propionic acid methyl ester (5.00 g, 11.9 mmol) in tetrahydrofuran (50 ml) was added methylmagnesium bromide (2.0M in ether, 18.0 ml, 36.0 mmol) at room temperature and the mixture was stirred for 20 minutes. The mixture was poured into a saturated aqueous solution of ammonium chloride (100 ml) and extracted with ethyl acetate (100 ml×1). The extract was washed with water (100 ml×2), brine (100 ml×1), dried (magnesium sulfate), and evaporated to give (S)-3-dibenzylamino-4-[4-(methoxymethoxy)phenyl]-2-methyl-butan-2-ol (5.03 g, 101%) as a yellow oil.

IR (Neat): 2968, 1608, 1510, 1234, 1153, 1005 cm⁻¹

NMR (CDCl₃, δ): 1.00 (3H, s), 1.26 (3H, s), 2.79-3.13 (3H, m), 3.34 (2H, br), 3.51 (3H, s), 3.91 (2H, br), 4.20 (1H, br), 5.20 (2H, s), 7.03-7.08 (2H, m), 7.22-7.36 (12H, m)

MS (m/z): 420 (M+1)

Preparation 81

To a solution of (S)-3-dibenzylamino-4-[4-(methoxymethoxy)phenyl]-2-methyl-butan-2-ol (1.00 g, 2.38 mmol) in methanol (10 ml) was added palladium (10% on activated carbon, 50% wet, 500 mg) and the mixture was hydrogenated (1 atm) for 6 hours. The catalyst was removed by filtration using Celite and washed with methanol. The filtrate was concentrated in vacuo to give (S)-3-amino-4-[4-(methoxymethoxy)phenyl]-2-methyl-butan-2-ol (555 mg, 97%) as a yellow solid.

MS (m/z): 240 (M+1)

Preparation 82

Potassium powder (85% purity, 236 mg, 3.58 mmol) was added to dimethyl sulfoxide (10 ml) at room temperature and the mixture was stirred for 1 hour. To the mixture was added (S)-4-(4-hydroxybenzyl)-2,2-dimethyloxazolidine-3-carboxylic acid tert-butyl ester (1.00 g, 3.25 mmol) and stirred for 10 minutes. Further, 4,7-dichloroquinoline (708 mg, 3.57 mmol) was added and the mixture was stirred at 100° C. for 5.5 hours. After cooling to room temperature, the mixture was quenched by the addition of water (20 ml) and extracted with ethyl acetate (20 ml×2). The combined extracts were washed with water (40 ml×2), brine (40 ml×1), dried (magnesium sulfate), and evaporated to give crude paste (1.44 g) as a yellow paste. The paste was purified by a recycling preparative HPLC equipped with a GPC column (eluent: chloroform) to give (S)-4-[4-(7-chloroquinolin-4-yloxy)-benzyl]-2,2-dimethyloxazolidine-3-carboxylic acid tert-butyl ester (1.42 g, 93%) as a pale yellow solid.

IR (KBr): 2978, 1697 (C═O), 1566, 1389, 1211 cm⁻¹

NMR (CDCl₃, δ): 1.49-1.67 (15H, m), 2.70-2.82 (1H, m), 3.11-3.28 (1H, m), 3.78-4.13 (3H, m), 6.54 (1H, d, J=5.2 Hz), 7.10-7.14 (2H, m), 7.33-7.38 (2H, m), 7.53 (1H, dd, J=2.0, 8.9 Hz), 8.08 (1H, d, J=2.0 Hz), 8.30 (1H, d, J=8.9 Hz), 8.66 (1H, d, J=5.2 Hz)

MS (m/z): 469 (M+1)

Preparation 83

To a solution of (S)-4-[4-(7-chloroquinolin-4-yloxy)-benzyl]-2,2-dimethyloxazolidine-3-carboxylic acid tert-butyl ester (1.31 g, 2.79 mmol) in a mixed solvent of dioxane (6.5 ml) and methanol (6.5 ml) was added 4N hydrogen chloride in dioxane (13 ml) at room temperature and the solution was stirred at the same temperature for 3 hours. The solvent was removed by evaporation to give, (S)-2-amino-3-[4-(7-chloroquinolin-4-yloxy)phenyl]propan-1-ol hydrochloride (1.47 g, 144%) as a pale yellow solid.

NMR (DMSO-d₆, δ): 2.95-2.99 (2H, m), 3.43-3.63 (3H, m), 4.77 (1H, br), 6.94 (1H, d, J=6.1 Hz), 7.37 (2H, d, J=8.5 Hz), 7.52 (2H, d, J=8.5 Hz), 7.93 (1H, dd, J=1.9, 9.0 Hz), 8.18 (3H, br s), 8.39 (1H, d, J=1.9 Hz), 8.53 (1H, d, J=9.0 Hz), 9.00 (1H, d, J=6.1 Hz)

MS (m/z): 329 (M−Cl⁻)

Preparation 84

To a solution of (S)-4-(2-benzylamino-3-hydroxypropyl)-phenol (4.00 g, 15.5 mmol) in ethanol (80 ml) was added (S)-3-phenoxy-1,2-epoxypropane (2.56 g, 17.0 mmol) and the solution was refluxed for 7 hours. After cooling to room temperature, the solvent was removed by evaporation and the residue was chromatographed on a 350 g of silica gel (eluent: chloroform/methanol=9/1) to give 4-[(2S)-2-[benzyl[(2S)-2-hydroxy-3-phenoxypropyl]amino]-3-hydroxypropyl]phenol (4.89 g, 77%) as a white foam.

NMR (CDCl₃, δ): 1.67 (2H, br), 2.46 (1H, dd, J=8.9, 13.7 Hz), 2.75-2.97 (4H, m), 3.04-3.16 (1H, m), 3.45-3.57 (2H, m), 3.66 (0.1H, d, J=13.5 Hz), 3.74-3.90 (3H, m), 3.92 (1H, d, J=13.5 Hz), 6.68 (2H, d, J=8.4 Hz), 6.81 (2H, d, J=7.8 Hz), 6.92-6.98 (1H, m), 6.94 (2H, d, J=8.4 Hz), 7.20-7.34 (7H, m)

MS (m/z): 408 (M+1)

Preparation 85

A mixture of (S)-[1-hydroxymethyl-2-(4-hydroxyphenyl)-ethyl]carbamic acid tert-butyl ester (1078 mg), 2,6-dibromopyridine (1110 mg), sodium tert-butoxide (0.7 g) and N,N-dimethylformamide (10 ml) was heated at 120° C. for 2 hours. To the reaction mixture, water (50 ml) and ethyl acetate (50 ml) was added. The organic layer was washed with water (50 ml×2 times) followed by brine (50 ml×1 time), dried over magnesium sulfate and evaporated to give (S)-4-[4-(6-bromo-2-pyridinyloxy)benzyl]-2-oxazolidinone as a crude product. To the product, methanol (10 ml), ammonium formate (4 g) and palladium on charcoal (0.2 g) were added, then the resulting mixture was heated under reflux for 30 minutes, filtered and evaporated to afford (S)-4-[4-(2-pyridinyloxy)-benzyl]-2-oxazolidinone as a crude product. The mixture of the product, ethanol (10 ml) and aqueous sodium hydroxide solution (3N, 7 ml) was heated at 90° C. for 1.5 hours, cooled to room temperature, added to hydrochloric acid solution (3N, 6 ml), and followed by the addition of ethyl acetate (50 ml). The organic layer was separated, washed with water (50 ml×2 times) followed by washing with brine (50 ml×1), dried over sodium sulfate and evaporated. The crude residue was purified by column chromatography (silica gel, dichloromethane:methanol:concentrated ammonia solution=7:1:0.1) to afford (S)-2-amino-3-[4-(2-pyridinyloxy)phenyl]-propanol (471 mg).

MS (m/z): 245 (M⁺+1)

Preparation 86

To a mixture of (S)-2-amino-3-[4-(2-pyridinyloxy)-phenyl]propanol (227 mg), dichloromethane (5 ml), benzaldehyde (108 mg) and acetic acid (0.11 ml), sodium triacetoxyborohydride (300 mg) was added and the mixture was stirred at room temperature overnight. The reaction mixture was poured into saturated aqueous sodium bicarbonate solution (30 ml), and extracted with ethyl acetate (20 ml×2 times). The organic layer was washed with water (20 ml×2 times) followed by washing with brine (20 ml×1 time), dried over sodium sulfate and evaporated to afford (S)-2-benzylamino-3-[4-(2-pyridinyloxy)phenyl]propanol (320 mg).

MS (m/z): 335 (M⁺+1)

Preparation 87

(S)-2-Amino-3-[4-(3-hydroxymethyl-2-pyridinyloxy)-phenyl]propanol dihydrochloride (350 mg) was hydrogenated by a similar manner to that described in Example 92 followed by free basing to afford (S)-2-amino-3-[4-(3-methyl-2-pyridinyloxy)phenyl]propanol (187 mg).

MS (m/z): 259 (M⁺+1)

Preparation 88

The following compound was synthesized according to a similar manner to that of Preparation 31.

(S)-{2-[4-(3-Formylquinolin-2-yloxy)phenyl]-1-hydroxymethylethyl}carbamic acid tert-butyl ester (11.74 g) as a Colorless Form

MALDI-MS (m/z): 425 (M+Na)

Preparation 89

To a mixture of (S)-{2-[4-(3-formylquinolin-2-yloxy)phenyl]-1-hydroxymethylethyl}carbamic acid tert-butyl ester (10.7 g), 35% hydrogen peroxide (5.5 ml) and potassium dihydrogenphosphate (13.85 g) in a mixture of acetonitrile (120 ml) and water (30 ml) was dropwise added sodium chlorite (80% purity, 8.63 g) at room temperature, and the mixture was stirred at the same temperature for 1 hour. While cooling in ice-water bath, to the mixture was added sodium sulfite (3.5 g). After removal of the bath, to this was added aqueous 1M citric acid to make it acidic, and extracted with ethyl acetate. The organic layer was washed with water and brine, dried over sodium sulfate, and evaporated in vacuo. The crude product was triturated with diisopropyl ether to give (S)-2-[4-(2-tert-butoxycarbonylamino-3-hydroxypropyl)-phenoxy]quinoline-3-carboxylic acid (4.8 g) as a colorless form.

MS (m/z): 439 (M+1)

Preparation 90

The following compound was synthesized according to a similar manner to that of Preparation 26.

(S)-2-[4-(2-tert-Butoxycarbonylamino-3-hydroxypropyl)-phenoxy]quinoline-3-carboxylic acid methyl ester (10.25 g) as a Colorless Form

MS (m/z): 453 (M+1)

Preparation 91

The following compound was synthesized according to a similar manner to that of Preparation 32.

(S)-2-[4-(2-Amino-3-hydroxypropyl)phenoxy]quinoline-3-carboxylic acid methyl ester hydrochloride (13.37 g) as a Colorless Powder

MS (m/z): 353 (M+1)

Preparation 92

The following compound was synthesized according to a similar manner to that of Example 41.

2-14-[(2S)-3-hydroxy-2-((2S)-2-hydroxy-3-phenoxy-propylamino)propyl]phenoxy}quinoline-3-carboxylic acid methyl ester (500 mg) as a Colorless Powder

MS (m/z): 503 (M+1)

Preparation 93

A mixture of {(1S)-1-hydroxymethyl-2-[4-(3-nitropyridin-2-yloxy)phenyl]ethyl}-(2S)-(2-hydroxy-3-phenoxypropyl)-carbamic acid tert-butyl ester (100 mg), 10% palladium on activated carbon (50% wet, 20 mg) and methanol (2.0 ml) was stirred at room temperature in the presence of hydrogen at an atmospheric pressure for 1 hour, and filtered. The filtrate was evaporated in vacuo to give {(1S)-1-hydroxymethyl-2-[4-(3-aminopyridin-2-yloxy)phenyl]ethyl}-(2S)-(2-hydroxy-3-phenoxypropyl)carbamic acid tert-butyl ester (1.1 g) as a brown oil.

MS (m/z): 510 (M+1)

### EXAMPLE 1

Under nitrogen, a solution of (S)-2-[4-(2-amino-3-hydroxypropyl)phenoxy]nicotinic acid methyl ester dihydrochloride (4.9 g), (R)-3-chlorostyrene oxide (5.0 g) and N,N-diisopropylethylamine (4.5 ml) in a mixture of methanol (10 ml) and 1,4-dioxane (10 ml) was refluxed for 28 hours. The mixture was evaporated in vacuo. The residue was purified by column chromatography on silica gel (dichloromethane:methanol=20:1), followed by treatment with oxalic acid in methanol and crystallization from methanol-ethyl acetate to give 2-[4-[(2S)-2-[(2R)-2-(3-chlorophenyl)-2-hydroxyethylamino]-3-hydroxypropyl]phenoxy]nicotinic acid methyl ester oxalate (1:1) (1.5 g).

NMR (DMSO-d₆, δ): 2.75-2.95 (1H, m), 3.05-3.20 (2H, m), 3.25-3.53 (3H, m), 3.55-3.70 (1H, m), 3.85 (3H, s), 4.95-5.05 (1H, m), 7.08 (2H, d, J=8.5 Hz), 7.20-7.50 (7H, m), 8.25-8.32 (2H, m)

### EXAMPLE 2

A mixture of 2-[4-[(2S)-2-[(2R)-2-(3-chlorophenyl)-2-hydroxyethylamino]-3-hydroxypropyl]phenoxy]nicotinic acid methyl ester oxalate (1:1) (0.65 g) and aqueous 28% ammonium hydroxide (26 ml) in 1,4-dioxane (13 ml) was stirred at room temperature for 2 days. The mixture was evaporated in vacuo, followed by partition between ethyl acetate and water. The organic layer was washed with brine, dried over sodium sulfate and evaporated in vacuo. The residue was treated with oxalic acid in ethanol followed by crystallization from methanol-ethyl acetate to give 2-[4-[(2S)-2-[(2R)-2-(3-chlorophenyl)-2-hydroxyethylamino]-3-hydroxypropyl]phenoxy]-nicotinamide oxalate (1:1) (0.41 g).

NMR (DMSO-d₆, δ): 2.75-2.95 (1H, m), 3.05-3.55 (5H, m), 3.55-3.70 (1H, m), 4.95-5.05 (1H, m), 7.1-7.6 (9H, m), 8.1-8.2 (2H, m)

### EXAMPLE 3

To a solution of 2-[4-[(2S)-2-[(2R)-2-(3-chlorophenyl)-2-hydroxyethylamino]-3-hydroxypropyl]phenoxy]nicotinic acid methyl ester oxalate (1:1) (0.71 g) in methanol (7.1 ml) was added with aqueous 1N sodium hydroxide (5.2 ml), and the mixture was stirred at room temperature for 2.5 hours. The mixture was evaporated in vacuo. The residue was dissolved in water followed by making acid at about pH 4 with aqueous 1N hydrogen chloride. After stirred for 8 hours, the precipitate was collected and recrystallized from ethyl acetate-methanol to give 2-[4-[(2S)-2-[(2R)-2-(3-chlorophenyl)-2-hydroxyethylamino]-3-hydroxypropyl]phenoxy]-nicotinic acid (0.52 g).

NMR (DMSO-d₆, δ): 2.55-2.75 (1H, m), 2.8.0-3.15 (4H, m), 3.20-3.35 (1H, m), 3.45-3.55 (1H, m), 4.85-4.95 (1H, m), 6.90 (2H, d, J=8.5 Hz), 7.10-7.20 (3H, m), 7.30-7.50 (4H, m), 8.05-8.20 (2H, m)

### EXAMPLE 4

Under nitrogen, a solution of (S)-2-[4-(2-amino-3-hydroxypropyl)phenoxy]nicotinonitrile hydrochloride (1.5 g), (R)-3-chlorostyrene oxide (1.5 g) and N,N-diisopropylethylamine (1.6 ml) in ethanol (9.6 ml) was refluxed for 4 hours. The mixture was evaporated in vacuo. The residue was purified by column chromatography on silica gel (dichloromethane:methanol=25:1), followed by treatment with 4N hydrogen chloride in ethyl acetate and trituration with ethyl acetate to give 2-[4-[(2S)-2-[(2R)-2-(3-chlorophenyl)-2-hydroxyethylamino]-3-hydroxypropyl]phenoxy]-nicotinitrile hydrochloride (0.96 g).

NMR (DMSO-d₆, δ): 2.8-3.8 (7H, m), 5.05-5.15 (1H, m), 7.22 (2H, d, J=8.4 Hz), 7.25-7.50 (7H, m), 8.36-8.45 (2H, m)

### EXAMPLE 5

Under nitrogen, a solution of 2-[4-[(2S)-2-[(2R)-2-(3-chlorophenyl)-2-hydroxyethylamino]-3-hydroxypropyl]phenoxy]-nicotinonitrile hydrochloride (1.0 g), di-tert-butyl dicarbonate (0.69 g) and triethylamine (0.89 ml) in N,N-dimethylformamide (10 ml) was stirred at room temperature for 9 hours. The mixture was diluted with ethyl acetate and poured into water. The organic layer was washed successively with aqueous 10% potassium hydrogen sulfate and brine, dried over sodium sulfate and evaporated in vacuo. The residue was purified by column chromatography on silica gel (hexane:ethyl acetate=3:2) to give [(2R)-2-(3-chlorophenyl)-2-hydroxyethyl][(1S)-1-[4-(3-cyanopyridin-2-yloxy)benzyl]-2-hydroxyethyl]carbamic acid tert-butyl ester (1.1 g).

NMR (DMSO-d₆, δ): 1.3-1.5 (9H, m)., 2.5-3.1 (3H, m), 3.2-3.9 (4H, m), 4.6-4.9 (1H, m), 7.13 (2H, d, J=8.5 Hz), 7.15-7.20 (7H, m), 8.3-8.35 (1H, m), 8.40 (1H, dd, J=1.6, 7.6 Hz)

### EXAMPLE 6

Under nitrogen, to a solution of a [(2R)-2-(3-chlorophenyl)-2-hydroxyethyl][(1S)-1-[4-(3-cyanopyridin-2-yloxy)benzyl]-2-hydroxyethyl]carbamic acid tert-butyl ester (0.50 g) in toluene (5 ml) was added diisopropylaluminum hydride (1M in hexane, 3.0 ml) at −78° C., and the mixture was stirred at the same temperature for 20 minutes. After quenched with aqueous 1M Rochelle salt, the mixture was extracted with ethyl acetate. The organic layer was washed with brine, dried over sodium sulfate, and evaporated in vacuo. Because of the occurrence of an unfavorable deprotection in most of the obtained products, the residue was treated with di-tert-butyl dicarbonate (0.61 g) and triethylamine (0.39 ml) in N,N-dimethylformamide (6 ml). After stirred at room temperature for 3 hours, the mixture was poured into aqueous 10% potassium hydrogen sulfate and extracted with ethyl acetate. The organic layer was washed with brine, dried over sodium sulfate, and evaporated in vacuo. The residue was purified by column chromatography on silica gel (hexane:ethyl acetate=3:2) to give [(2R)-2-(3-chlorophenyl)-2-hydroxyethyl][(1S)-1-[4-(3-formylpyridin-2-yloxy)benzyl]-2-hydroxyethyl]carbamic acid tert-butyl ester (0.26 g).

NMR (CDCl₃, δ): 1.54 (9H, s), 2.3-3.1 (3H, m), 3.1-3.9 (4H, m), 4.6-4.9 (1H, m), 6.9-7.4 (9H, m), 8.19-8.23 (2H, m), 10.53 (1H, s)

### EXAMPLE 7

Under nitrogen, to a solution of [(2R)-2-(3-chlorophenyl)-2-hydroxyethyl][(1S)-1-[4-(3-formylpyridin-2-yloxy)benzyl]-2-hydroxyethyl]carbamic acid tert-butyl ester (0.24 g) in methanol (4.5 ml) was added sodium borohydride (17 mg) at 5° C., and the mixture was stirred at the same temperature for 10 minutes. The mixture was evaporated in vacuo. To the residue was added water and extracted with ethyl acetate. The organic layer was washed with brine, dried over sodium sulfate, and evaporated in vacuo. The residue was purified by column chromatography on silica gel (hexane:ethyl acetate=3:2) to give [(2R)-2-(3-chlorophenyl)-2-hydroxyethyl][(1S)-2-hydroxy-1-[4-(3-hydroxymethylpyridin-2-yloxy)benzyl]ethyl]carbamic acid tert-butyl ester (0.15 g).

NMR (CDCl₃, δ): 1.53 (9H, s), 2.3-3.05 (3H, m), 3.2-3.9 (4H, m), 4.75-4.90 (3H, m), 6.94-7.25 (9H, m), 7.70-7.80 (1H, m), 7.90-7.95 (1H, m)

### EXAMPLE 8

To a solution of [(2R)-2-(3-chlorophenyl)-2-hydroxyethyl][(1S)-2-hydroxy-1-[4-(3-hydroxymethylpyridin-2-yloxy)benzyl]ethyl]carbamic acid tert-butyl ester (0.14 g) in ethyl acetate (5.2 ml) was added 4N hydrogen chloride in ethyl acetate (1.3 ml) and the resulting mixture was allowed to stand at room temperature for 2 hours. The mixture was evaporated in vacuo. To the residue was added aqueous saturated sodium bicarbonate and extracted with ethyl acetate. The organic layer was washed with brine, dried over sodium sulfate, and evaporated in vacuo. The residue was purified by column chromatography on silica gel (dichloromethane:methanol=10:1), followed by treatment with oxalic acid in ethanol and trituration with ethyl acetate to give (2S)-2-[(2R)-2-(3-chlorophenyl)-2-hydroxyethylamino]-3-[4-(3-hydroxymethylpyridin-2-yloxy)phenyl]propan-1-ol oxalate (1:1) (83 mg).

NMR (DMSO-d₆, δ): 2.75-2.90 (1H, m), 3.0-3.5 (5H, m), 3.55-3.70 (1H, m), 4.62 (2H, s), 4.90-5.05 (1H, m), 7.04-7.17 (3H, m), 7.27-7.48 (6H, m), 7.87-7.98 (2H, m)

### EXAMPLE 9

Under nitrogen, a solution of (S)-6-[4-(2-amino-3-hydroxypropyl)phenoxy]nicotinic acid methyl ester dihydrochloride (1.3 g), (R)-3-chlorostyrene oxide (1.7 g) and N,N-diisopropylethylamine (1.2 ml) in a mixture of methanol (2.7 ml) and 1,4-dioxane (2.7 ml) was refluxed for 16 hours. The mixture was evaporated in vacuo. The residue was dissolved in ethyl acetate, washed successively with water and brine, dried over sodium sulfate, and evaporated in vacuo. The residue was purified by column chromatography on silica gel (dichloromethane:methanol=25:1) to give 6-[4-[(2S)-2-[(2R)-2-(3-chlorophenyl)-2-hydroxyethylamino]-3-hydroxypropyl]phenoxy]nicotinic acid methyl ester (0.83 g). The crude product was used in the next step.

### EXAMPLE 10

A solution of 6-[4-[(2S)-2-[(2R)-2-(3-chlorophenyl)-2-hydroxyethylamino]-3-hydroxypropyl]phenoxy]nicotinic acid methyl ester (0.60 g) in methanol (6 ml) was treated with 4N hydrogen chloride in 1,4-dioxane (1 ml), and evaporated in vacuo. The crude product was crystallized from methanol-ethyl acetate and collected to give 6-[4-[(2S)-2-[(2R)-2-(3-chlorophenyl)-2-hydroxyethylamino]-3-hydroxypropyl]phenoxy]-nicotinic acid methyl ester dihydrochloride (0.33 g).

NMR (DMSO-d₆, δ): 2.8-3.0 (1H, m), 3.1-3.75 (6H, m), 3.86 (3H, s), 5.05-5.1 (1H, m), 7.1-7.2 (3H, m), 7.35-7.55 (6H, m), 8.32 (1H, dd, J=2.4, 8.7 Hz), 8.69 (1H, d, J=1.9 Hz)

### EXAMPLE 11

A mixture of 6-[4-[(2S)-2-[(2R)-2-(3-chlorophenyl)-2-hydroxyethylamino]-3-hydroxypropyl]phenoxy]nicotinic acid methyl ester dihydrochloride (0.26 g) and aqueous 28% ammonium hydroxide (26 ml) in 1,4-dioxane (5.2 ml) was stirred at room temperature for 1 day. The mixture was evaporated in vacuo, followed by partition between ethyl acetate and water. The organic layer was washed with brine, dried over sodium sulfate and evaporated in vacuo to give 6-[4-[(2S)-2-[(2R)-2-(3-chlorophenyl)-2-hydroxyethylamino]-3-hydroxypropyl]phenoxy]nicotinamide (0.21 g).

NMR (DMSO-d₆, δ): 2.5-2.9 (5H, m), 3.1-3.4 (2H, m), 4.55-4.65 (1H, m), 7.00-7.06 (3H, m), 7.20-7.50 (6H; m), 8.25 (1H, dd, J=2.5, 8.6 Hz), 8.62 (1H, d, J=2.2 Hz)

### EXAMPLE 12

Crude 6-[4-[(2S)-2-[(2R)-2-(3-chlorophenyl)-2-hydroxyethylamino]-3-hydroxypropyl]phenoxy]nicotinamide (0.31 g) was purified by column chromatography on silica gel (dichloromethane:methanol=10:1), followed by crystallization from ethanol-ethyl acetate to pure 6-[4-[(2S)-2-[(2R)-2-(3-chlorophenyl)-2-hydroxyethylamino]-3-hydroxypropyl]phenoxy]nicotinamide (90 mg). 6-[4-[(2S)-2-((2R)-2-(3-Chlorophenyl)-2-hydroxyethylamino]-3-hydroxypropyl]phenoxy]nicotinamide was treated with 4N hydrogen chloride in ethyl acetate, followed by crystallization from methanol-ethyl acetate to give 6-[4-[(2S)-2-[(2R)-2-(3-chlorophenyl)-2-hydroxyethylamino]-3-hydroxypropyl]phenoxy]nicotinamide dihydrochloride (57 mg).

NMR (DMSO-d₆, δ): 2.8-3.7 (7H, m), 5.0-5.1 (1H, m), 7.06-7.20 (3H, m), 7.30-7.50 (6H, m), 8.27 (1H, dd, J=2.5, 8.6 Hz), 8.61 (1H, d, J=2.4 Hz)

### EXAMPLE 13

To a solution of 6-[4-[(2S)-2-[(2R)-2-(3-chlorophenyl)-2-hydroxyethylamino]-3-hydroxypropyl]phenoxy]nicotinic acid methyl ester dihydrochloride (0.18 g) in methanol (3.6 ml) was added with aqueous 1N sodium hydroxide (1.4 ml), and the mixture was stirred at room temperature for 4.5 hours. To the mixture was added aqueous 1N hydrogen chloride (1.2 ml) and evaporated in vacuo. After partition between water and ethyl acetate contained a little amount of methanol, the organic layer was washed with brine, dried over sodium sulfate, and evaporated in vacuo. The residue was treated with 4N hydrogen chloride in 1,4-dioxane, followed by evaporation and trituration with ethyl acetate to give 6-[4-[(2S)-2-[(2R)-2-(3-chlorophenyl)-2-hydroxyethylamino]-3-hydroxypropyl]phenoxy]nicotinic acid dihydrochloride (0.1 g).

NMR (DMSO-d₆, δ): 2.8-3.8 (7H, m), 5.05-5.15 (1H, m), 7.08-7.20 (3H, m), 7.35-7.50 (6H, m), 8.29 (1H, dd, J=2.1, 8.5 Hz), 8.66 (1H, d, J=2.3 Hz)

### EXAMPLE 14

Under nitrogen, a solution of (S)-2-amino-3-[4-(3,5-dichloropyridin-4-yloxy)phenyl]propan-1-ol hydrochloride (0.39 g), (R)-3-chlorostyrene oxide (0.50 g) and N,N-diisopropylethylamine (0.19 ml) in a mixture of methanol (1 ml) and 1,4-dioxane (1 ml) was refluxed for 14.5 hours. The mixture was evaporated in vacuo. The residue was dissolved in ethyl acetate, washed successively with water and brine, dried over sodium sulfate, and evaporated in vacuo. The residue was purified by column chromatography on silica gel (dichloromethane:methanol=15:1), followed by treatment with 4N hydrogen chloride in ethyl acetate and crystallization from methanol-ethyl acetate to give (2S)-2-[(2R)-2-(3-chlorophenyl)-2-hydroxyethylamino]-3-[4-(3,5-dichloropyridin-4-yloxy)phenyl]propan-1-ol dihydrochloride (0.13 g).

NMR (DMSO-d₆, δ): 2.75-2.9 (1H, m), 2.9-3.5 (5H, m), 3.5-3.7 (1H, m), 5.0-5.1 (1H, m), 6.91 (2H, d, J=8.6 Hz), 7.3-7.5 (6H, m), 8.79 (2H, s)

### EXAMPLE 15

The following compound was obtained according to a similar manner to that of Example 1.

(2S)-2-[(2R)-2-(3-Chlorophenyl)-2-hydroxyethylamino]-3-[4-(6-fluoropyridin-2-yloxy)phenyl]propan-1-ol oxalate (1:1)

NMR (DMSO-d₆, δ): 2.75-2.95 (1H, m), 3.0-3.5 (5H, m), 3.55-3.70 (1H, m), 4.9-5.1 (1H, m), 6.85-6.95 (2H, m), 7.15 (2H, d, J=8.5 Hz), 7.3-7.5 (6H, m), 8.02 (1H, q, J=8.0 Hz)

### EXAMPLE 16

Under nitrogen, a suspension of (S)-4-[4-(6-chloropyridin-2-yloxy)benzyl]oxazolidin-2-one (0.49 g), (R)-3-chlorostyrene oxide (0.49 g) and potassium carbonate (0.44 g) in N,N-dimethylformamide (4.9 ml) was stirred at 80° C. for 96 hours. The mixture was diluted with ethyl acetate and insoluble materials were filtered off. The filtrate was evaporated in vacuo. The residue was dissolved in a mixture of ethanol (8 ml) and water (7 ml). To it was aqueous 4N sodium hydroxide (5.6 ml), and the mixture was refluxed for 3 hours. After evaporation in vacuo and partition between ethyl acetate and water, the organic layer was washed with brine, dried over sodium sulfate, and evaporated in vacuo. The residue was purified by column chromatography on silica gel (dichloromethane:methanol=25:1), followed by treatment with oxalic acid in ethanol and trituration with ethyl acetate to give (2S)-2-[(2R)-2-(3-chlorophenyl)-2-hydroxyethylamino]-3-[4-(6-chloropyridin-2-yloxy)phenyl]-propan-1-ol oxalate (2:1) (62 mg).

NMR (DMSO-d₆, δ): 2.7-3.6 (7H, m), 4.8-4.9 (1H, m), 6.98 (1H, d, J=8.1 Hz), 7.11 (2H, d, J=8.4 Hz), 7.2-7.5 (7H, m), 7.90 (1H, t, J=8.0 Hz)

### EXAMPLE 17

To a solution of (5R)-5-(3-chlorophenyl)-3-{(1S)-2-hydroxy-1-[4-(pyridin-2-yloxy)benzyl]ethyl}oxazolidin-2-one (0.22 g) in ethanol (5.1 ml) was added aqueous 4N sodium hydroxide (1.5 ml), and the mixture was refluxed for 2 hours. The mixture was evaporated in vacuo. After partition between ethyl acetate and water, the organic layer was washed with brine, dried over sodium sulfate, and evaporated in vacuo. The residue was purified by column chromatography on silica gel (chloroform:methanol=25:1), followed by treatment with 4N hydrogen chloride in ethyl acetate and trituration with ethyl acetate to give (2S)-2-[(2R)-2-(3-chlorophenyl)-2-hydroxyethylamino]-3-[4-(pyridin-2-yloxy)phenyl]propan-1-ol dihydrochloride (0.15 g).

NMR (DMSO-d₆, δ): 2.75-3.0 (1H, m), 3.05-3.7 (6H, m), 5.0-5.2 (1H, m), 7.0-7.2 (4H, m), 7.25-7.55 (6H, n), 7.8-7.9 (1H, m), 8.1-8.2 (1H, m)

### EXAMPLE 18

To a solution of (4S)-3-[(2R)-2-(3-chlorophenyl)-2-hydroxyethyl]-4-[4-(pyridin-2-yloxy)benzyl]oxazolidin-2-one (0.67 g) in ethanol (6.7 ml) was added aqueous 4N sodium hydroxide (4 ml), and the mixture was refluxed for 2 hours. After evaporation in vacuo and partition between water and ethyl acetate, the organic layer was washed with brine, dried over sodium sulfate, and evaporated in vacuo. The residue was dissolved in N,N-dimethylformamide (9 ml), and to it were added triethylamine (0.48 ml)) and a solution of di-tert-butyl dicarbonate (0.63 g) in N,N-dimethylformamide (3 ml). After stirred for 5 hours, the mixture was poured into aqueous 10% potassium hydrogen sulfate, and extracted with ethyl acetate. The organic layer was washed with brine, dried over sodium sulfate, and evaporated in vacuo. The residue was purified by column chromatography on silica gel (dichloromethane:methanol=50:1) to give [(2R)-2-(3-chlorophenyl)-2-hydroxyethyl][(2S)-2-hydroxy-1-[4-(pyridin-2-yloxy)benzyl)ethyl]carbamic acid tert-butyl ester (0.85 g).

NMR (CDCl₃, δ): 1.53 (9H, s), 2.3-4.5 (7H, m), 4.8-4.9 (1H, m), 6.83 (1H, d, J=8.2 Hz), 6.9-7.5 (8H, m), 7.55-7.7 (1H, m), 8.01 (1H, s), 8.05-8.15 (1H, m)

### EXAMPLE 19

Under nitrogen, to a solution of [(2R)-2-(3-chlorophenyl)-2-hydroxyethyl][(2S)-2-hydroxy-1-[4-(pyridin-2-yloxy)benzyl]ethyl]carbamic acid tert-butyl ester (0.82 g) in dichloromethane (8.2 ml) was added 3-chloroperbenzoic acid (1.0 g), and the mixture was stirred at room temperature for 1 day. The mixture was diluted with ethyl acetate, washed with aqueous 1N sodium hydroxide and brine, dried over sodium sulfate, and evaporated in vacuo. The residue was purified by column chromatography on silica gel (dichloromethane:methanol=25:1) to give [(2R)-2-(3-chlorophenyl)-2-hydroxyethyl][(2S)-2-hydroxy-1-[4-(1-oxypyridin-2-yloxy)benzyl]ethyl]carbamic acid tert-butyl ester (0.64 g).

NMR (CDCl₃, δ): 1.53 (9H, s), 2.1-4.6 (7H, m), 4.8-5.2 (1H, m), 6.65-6.8 (1H, m), 6.95-7.4 (10H, m), 8.25-8.35 (1H, m)

### EXAMPLE 20

The following compound was obtained according to a similar manner to that of Example 8.

(2R)-2-[(2S)-2-(3-Chlorophenyl)-2-hydroxyethylamino]-3-[4-(1-oxypyridin-2-yloxy)phenyl]propan-1-ol oxalate (1:1)

NMR (DMSO-d₆, δ): 2.8-3.7 (7H, m), 4.9-5.1 (1H, m), 6.94 (2H, d, J=8.3 Hz), 7.15-7.55 (9H, m), 8.30-8.40 (1H, m)

### EXAMPLE 21

To a solution of (4S)-3-[(2R)-2-(3-chlorophenyl)-2-hydroxyethyl]-4-[4-(6-hydroxymethylpyridin-2-yloxy)benzyl]-oxazolidin-2-one (0.37 g) and (5R)-5-(3-chlorophenyl)-3-{(1S)-2-hydroxy-1-[4-(6-hydroxymethylpyridin-2-yloxy)benzyl]-ethyl}oxazolidin-2-one (62 mg) in ethanol (2.8 ml) was added aqueous 2N sodium hydroxide (5.6 ml), and the mixture was refluxed for 3 hours. The mixture was evaporated in vacuo. After partition between ethyl acetate and water, the organic layer was washed with brine, dried over sodium sulfate, and evaporated in vacuo. The residue was purified by column chromatography on silica gel (dichloromethane:methanol=10:1), followed by treatment with oxalic acid in ethanol and trituration with hexane to give (2S)-2-[(2R)-2-(3-chlorophenyl)-2-hydroxyethylamino]-3-[4-(6-hydroxymethylpyridin-2-yloxy)phenyl]propan-1-ol oxalate (1:1) (0.38 g).

NMR (DMSO-d₆, δ): 2.7-2.95 (1H, m), 3.0-3.7 (6H, m), 4.38 (2H, s), 5.01 (1H, d, J=8.0 Hz), 6.80 (1H, d, J=8.1 Hz), 7.08 (2H, d, J=8.4 Hz), 7.2-7.5 (7H, m), 7.84 (1H, t, J=7.6 Hz)

### EXAMPLE 22

The following compound was obtained according to a similar manner to that of Example 21 starting from (4S)-3-[(2R)-2-(3-chlorophenyl)-2-hydroxyethyl]-4-4-(5-hydroxymethylpyridin-2-yloxy)benzyl]oxazolidin-2-one.

(2S)-2-[(2R)-2-(3-Chlorophenyl)-2-hydroxyethylamino]-3-[4-(5-hydroxymethylpyridin-2-yloxy)phenyl]propan-1-ol oxalate (1:1)

NMR (DMSO-d₆, δ): 2.75-2.90 (1H, m), 3.0-3.55 (5H, m), 3.55-3.70 (1H, m), 4.48 (2H, s), 4.9-5.1 (1H, m), 6.98 (1H, d, J=8.4 Hz), 7.07 (2H, d, J=8.5 Hz), 7.31 (2H, d, J=8.5 Hz), 7.3-7.5 (4H, m), 7.80 (1H, dd, J=2.4, 8.4 Hz), 8.07 (1H, d, J=2.2 Hz)

### EXAMPLE 23

Under nitrogen, a solution of (S)-2-[4-(2-amino-3-hydroxypropyl)phenoxy]nicotinonitrile hydrochloride (5.0 g), (2S)-1,2-epoxy-3-phenoxypropane (2.45 g) and N,N-diisopropylethylamine (5.5 ml) in ethanol (50 ml) was refluxed for 7 hours. The mixture was evaporated in vacuo. The residue was purified by column chromatography on silica gel (chloroform:methanol=100:1) to give 2-[4-[(2S)-2-[(2S)-2-hydroxy-3-phenoxypropylamino]-3-hydroxypropyl]phenoxy]-nicotinonitrile (4.92 g).

IR (NaCl): 3380, 2240, 1590, 1427 cm⁻¹

NMR (CDCl₃, δ): 2.60-3.14 (7H, m), 3.50-3.80 (3H, m), 3.95 (2H, d, J=4.9 Hz), 4.00-4.17 (1H, m), 6.80-7.32 (10H, m), 8.00 (1H, dd, J=2.0, 7.6 Hz), 8.07 (1H, dd, J=2.0, 5.0 Hz)

### EXAMPLE 24

2-[4-[(2S)-2-[(2S)-2-Hydroxy-3-phenoxypropylamino]-3-hydroxypropyl]phenoxy]nicotinonitrile (73 mg) was treated with 4N hydrogen chloride in ethyl acetate (3 ml) and triturated with diisopropyl ether to give 2-[4-[(2S)-2-[(2S)-2-hydroxy-3-phenoxypropylamino]-3-hydroxypropyl]phenoxy]-nicotinonitrile hydrochloride (70 mg).

NMR (DMSO-d₆, δ): 2.60-3.14 (7H, m), 3.50-3.80 (3H, m), 3.95 (2H, d, J=4.9 Hz), 4.00-4.17 (1H, m), 6.80-7.32 (10H, m), 8.00 (1H, dd, J=2.0, 7.6 Hz), 8.07 (1H, dd, J=2.0, 5 Hz)

### EXAMPLE 25

Under nitrogen, a solution of 2-[4-[(2S)-2-[(2S)-2-hydroxy-3-phenoxypropylamino]-3-hydroxypropyl]phenoxy]-nicotinonitrile (3.0 g), di-tert-butyl dicarbonate (1.8 g) in tetrahydrofuran (20 ml) was stirred at room temperature for 9 hours. The mixture was evaporated in vacuo to give [(1S)-1-[4-(3-cyanopyridin-2-yloxy)benzyl]-2-hydroxyethyl][(2S)-2-hydroxy-3-phenoxypropyl]carbamic acid tert-butyl ester (3.0 g).

NMR (CDCl₃, δ): 1.46 (9H, s), 2.40-3.95 (7H, m), 4.00-4.40 (3H, m), 6.80-7.40 (10H, m), 8.00 (1H, dd, J=2.0, 7.5 Hz), 8.20 (1H, dd, J=2.0, 5.0 Hz)

### EXAMPLE 26

Under nitrogen, to a solution of a [(1S)-1-[4-(3-cyanopyridin-2-yloxy)benzyl]-2-hydroxyethyl][(2S)-2-hydroxy-3-phenoxypropyl]carbamic acid tert-butyl ester (1.50 g) in toluene (20 ml) was added diisopropylaluminum hydride (1M in hexane, 17.3 ml) at −78° C., and the mixture was stirred at the same temperature for 20 minutes. After quenched with aqueous 1M Rochelle salt, the mixture was extracted with ethyl acetate. The organic layer was washed with brine, dried over sodium sulfate, and evaporated in vacuo. Because of the occurrence of an unfavorable deprotection in most of the obtained products, the residue was treated with di-tert-butyl dicarbonate (0.6 g) and triethylamine (0.39 ml) in N,N-dimethylformamide (6 ml). After stirred at room temperature for 3 hours, the mixture was poured into aqueous 10% potassium hydrogen sulfate and extracted with ethyl acetate. The organic layer was washed with brine, dried over sodium sulfate, and evaporated in vacuo. The residue was purified by column chromatography on silica gel (hexane:ethyl acetate=1:2) to give [(1S)-2-[4-(3-formylpyridin-2-yloxy)benzyl]-2-hydroxyethyl][(2S)-2-hydroxy-3-phenoxypropyl]carbamic acid tert-butyl ester (0.80 g).

NMR (CDCl₃, δ): 1.46 (9H s), 2.70-3.90 (7H, m), 4.05-4.40 (3H, m), 6.80-7.40 (10H, m), 8.16-8.32 (2H, m), 10.54 (1H, s)

### EXAMPLE 27

Under nitrogen, a solution of [(1S)-1-[4-(3-cyanopyridin-2-yloxy)benzyl]-2-hydroxyethyl][(2S)-2-hydroxy-3-phenoxypropyl]carbamic acid tert-butyl ester (150 mg) in dimethyl sulfoxide (7.2 ml) was added 30% hydrogen peroxide (0.72 ml) and aqueous 5N sodium hydroxide (0.72 ml) at 0° C., and the mixture was stirred at the same temperature for 1 hour. The mixture was extracted with ethyl acetate. The organic layer was washed with brine, dried over sodium sulfate, and evaporated in vacuo to give [(1S)-1-[4-(3-carbamoylpyridin-2-yloxy)benzyl]-2-hydroxyethyl][(2S)-2-hydroxy-3-phenoxypropyl]carbamic acid tert-butyl ester (150 mg).

NMR (CDCl₃, δ): 1.42 (9H, s), 2.50-4.00 (7H, m), 4.05-4.40 (3H, m), 5.90 (1H, br s), 6.85-7.35 (10H, m), 7.73 (1H, br s), 8.18 (1H, dd, J=2.0, 4.8 Hz), 8.61 (1H, dd, J=2.0, 7.6 Hz)

### EXAMPLE 28

Under nitrogen, to a solution of [(1S)-1-[4-(3-formylpyridin-2-yloxy)benzyl]-1-hydroxyethyl][(2S)-2-hydroxy-3-phenoxypropyl]carbamic acid tert-butyl ester (273 mg) in methanol (5 ml) was added sodium borohydride (20 mg) at 5° C., and the mixture was stirred at the same temperature for 30 minutes. The mixture was evapoated in vacuo. To the residue was added water and extracted with ethyl aceate. The organic layer was washed with brine, dried over sodium sulfate, and evaporated in vacuo. The residue was purified by column chromatography on silica gel (chloroform:methanol=100:1) to give [(1S)-1-[4-(3-hydroxymethylpyridin-2-yloxy)-benzyl]-2-hydroxyethyl][(2S)-2-hydroxy-3-phenoxypropyl]-carbamic acid tert-butyl ester (260 mg).

NMR (DMSO-d₆, δ): 1.43 (9H, s), 2.60-3.05 (4H, m), 3.40-4.05 (6H, m), 4.60 (1H, d, J=5.5 Hz), 5.0 (1H, br s), 5.2 (1H, br s), 5.33 (1H, t, J=5.5 Hz), 6.86-7.01 (5H, m), 7.11-7.32 (5H, m), 7.87 (1H, dd, J=1.0, 7.3 Hz), 7.94 (1H, dd, J=1.9, 4.9 Hz)

### EXAMPLE 29

To a solution of [(1S)-1-[4-(3-carbamoylpyridin-2-yloxy)benzyl]-2-hydroxyethyl][(2R)-2-hydroxy-3-phenoxypropyl]carbamic acid tert-butyl ester (150 mg) in ethyl acetate (5 ml) was added 4N hydrogen chloride in ethyl acetate (5 ml) at room temperature, and the solution was stirred at the same temperature for 4 hours. The mixture was evaporated in vacuo, and the residue was triturated with diisopropyl ether to give 2-[4-[(2S)-2-[(2R)-2-hydroxy-3-phenoxypropylamino]-3-hydroxypropyl]phenoxy]nicotinamide hydrochloride (130 mg).

NMR (DMSO-d₆, δ): 2.80-3.80 (7H, m), 3.90-4.10 (2H, m), 4.20-4.30 (1H, m), 6.91-7.00 (3H, m), 7.18-7.40 (7H, m), 7.78 (2H, br s), 8.14-8.18 (2H, m), 8.53 (1H, br s), 9.15 (1H, br s)

### EXAMPLE 30

To a solution of [(1S)-1-[4-(3-hydroxymethylpyridin-2-yloxy)benzyl]-2-hydroxyethyl][(2S)-2-hydroxy-3-phenoxypropyl]carbamic acid tert-butyl ester (260 mg) in ethyl acetate (5 ml) was added 4N hydrogen chloride in ethyl acetate (5 ml) at room temperature, and the solution was stirred at the same temperature for 4 hours. The mixture was evaporated in vacuo, and the residue was triturated with diisopropyl ether to give (2S)-3-[4-(3-hydroxymethylpyridin-2-yloxy)phenyl]-2-[(2S)-2-hydroxy-3-phenoxypropylamino]-propan-1-ol hydrochloride (130 mg).

NMR (DMSO-d₆, δ): 2.85-3.85 (7H, m), 4.00 (2H, d, J=4.6 Hz), 4.20-4.35 (1H, m), 4.62 (2H, s), 6.90-7.20 (6H, m), 7.23-7.42 (4H, m), 7.90 (1H, d, J=7.5 Hz), 7.97 (1H, d, J=4.7 Hz), 8.51 (1H, br s), 9.10 (1H, br s)

### EXAMPLE 31

Under nitrogen, a solution of 2-[4-(2S)-(2-amino-3-hydroxypropyl)phenoxy]nicotinamide dihydrochloride (200 mg), (2S)-1,2-epoxy-3-(3-fluorophenoxy)propane (93 mg) and N,N-diisopropylethylamine (0.29 ml) in ethanol (10 ml) was refluxed for 7 hours. The mixture was evaporated in vacuo. The residue was purified by column chromatography on silica gel (chloroform:methanol=100:1), followed by treatment with 4N hydrogen chloride in dioxane to give 2-(4-{3-hydroxy-(2S)-2-[(2S)-2-hydroxy-3-(3-fluoro)phenoxypropylamino]propyl}-phenoxy)nicotinamide dihydrochloride (40 mg) as a colorless powder.

NMR (DMSO-d₆, δ): 2.80-2.95 (1H, m), 3.10-3.70 (6H, m), 4.03 (2H, d, J=4.9 Hz), 4.20-4.30 (1H, m), 6.70-6.90 (3H, m), 7.10-7.40 (6H, m), 7.78 (1H, br s), 8.10-8.20 (2H, m), 8.52 (1H, br s), 9.08 (1H, br s)

MS (m/z): 456 (M+1)

### EXAMPLE 32

The following compounds were synthesized according to a similar manner to that of Example 31.

(1) 2-(4-{3-Hydroxy-(2S)-2-[(2S)-2-hydroxy-3-(4-chloro)-phenoxypropylamino]propyl}phenoxy)nicotinamide dihydrochloride as a Colorless Powder

IR (KBr): 3560-3,300, 1695, 1668, 1652, 1419, 1241 cm⁻¹

NMR (DMSO-d₆, δ): 2.80-3.00 (1H, m), 3.10-3.80 (6H, m), 4.00 (2H, d, J=5.0 Hz), 4.20-4.30 (1H, m), 6.90-7.40 (9H, m), 7.78 (1H, br s), 8.10-8.20 (2H, m), 8.52 (1H, br s), 9.08 (1H, br s)

MS (m/z): 472 (M+1)

(2) 2-(4-{3-Hydroxy-(2S)-2-[(2S)-2-hydroxy-3-(2-fluoro)-phenoxypropylamino]propyl}phenoxy)nicotinamide dihydrochloride as a Colorless Powder

IR (KBr): 3760-3330, 1670, 1652, 1590, 1508, 1419 cm⁻¹

NMR (DMSO-d₆, δ): 2.90-3.05 (1H, m), 3.10-3.90 (7H, m), 4.05 (1H, d, J=4.9 Hz), 4.20-4.30 (1H, m), 6.90-7.07 (3H, m), 7.10-7.40 (6H, m), 7.78 (1H, br s), 8.10-8.20 (2H, m), 8.53 (1H, br s), 9.10 (1H, br s)

MS (m/z): 472 (M+1)

(3) (2S)-2-(4-{3-Hydroxy-2-[(2S)-2-hydroxy-3-(4-fluoro)-phenoxypropylamino]propyl}phenoxy)nicotinamide dihydrochloride (40 mg) as a Colorless Powder

IR (KBr): 3360-3050, 1683, 1650, 1650, 1508, 1419 cm⁻¹

NMR (DMSO-d₆, δ): 2.80-3.00 (1H, m), 3.10-3.80 (6H, m), 4.00 (2H, d, J=4.9 Hz), 4.20-4.40 (1H, m), 6.90-7.10 (2H, m), 7.15-7.30 (5H, m), 7.35-7.40 (2H, m), 7.78 (1H, br s), 8.10-8.20 (2H, m), 8.50-8.55 (2H, m), 8.78 (1H, br s), 9.07 (1H, br s)

MS (m/z): 456 (M+1)

(4) (2S)-3-[4-(3-Chloroquinoxalin-2-yloxy)phenyl]-2-((2S)-2-hydroxy-3-phenoxypropylamino)propan-1-ol (60 mg)

NMR (DMSO-d₆, δ): 2.55-2.85 (5H, m), 3.2-3.5 (2H, m), 3.75-4.0 (3H, m), 6.85-6.95 (3H, m), 7.2-7.4 (6H, m), 7.65-7.8 (3H, m), 7.95-8.05 (1H, m)

(5) (2S)-3-[4-(Benzothiazol-2-yloxy)phenyl]-2-((2S)-2-hydroxy-3-phenoxypropylamino)propan-1-ol (90 mg)

NMR (DMSO-d₆, δ): 2.55-2.9 (5H, m), 3.15-3.45 (2H, m), 3.75-4.0 (3H, m), 6.85-7.0 (3H, m), 7.2-7.5 (8H, m), 7.65-7.7 (1H, m), 7.85-7.95 (1H, m)

(6) (2S)-2-((2S)-2-Hydroxy-3-phenoxypropylamino)-3-[4-([1,7]naphthyridin-8-yloxy)phenyl]propan-1-ol (57 mg)

NMR (DMSO-d₆, δ): 2.5-2.9 (5H, m), 3.15-3.6 (2H, m), 3.8-4.05 (3H, m), 6.85-7.0 (3H, m), 7.12 (2H, d, J=8.5 Hz), 7.2-7.35 (4H, m), 7.57 (1H, d, J=5.7 Hz), 7.85 (1H, ABq, J=4.2, 8.3 Hz), 8.00 (1H, d, J=5.6 Hz), 8.44 (1H, ABq, J=1.7, 8.4 Hz), 9.05 (1H, ABq, J=1.7, 4.2 Hz)

(7) (2S)-2-((2S)-2-hydroxy-3-phenoxypropylamino)-3-[4-(6-nit roquinolin-4-yloxy)phenyl]propan-1-ol (80 mg).

NMR (DMSO-d₆, δ): 2.55-2.85 (5H, m), 3.2-3.5 (2H, m), 3.7-4.0 (3H, m), 6.71 (1H, d, J=5.3 Hz), 6.85-7.0 (3H, m), 7.2-7.35 (4H, m), 7.40 (2H, d, J=8.5 Hz), 8.25 (1H, d, J=9.3 Hz), 8.55 (1H, ABq, J=2.6, 9.3 Hz), 8.85 (1H, d, J=5.3 Hz), 9.15 (1H, d, J=2.6 Hz)

(8) 2(S)-2-[(2S)-2-hydroxy-3-(phenoxy)propylamino]-3-[4-(iso quinolin-1-yloxy)phenyl]propan-1-ol (67.2 mg, 49%) as a yellow crystalline solid.

IR (KBr): 3408, 1628, 1597, 1570, 1496, 1375, 1246, 1221 cm⁻¹

NMR (CDCl₃, δ): 2.17, 2.70-2.89 (5H, m), 3.48 (1H, dd, J=4.2, 10.8 Hz), 3.70 (1H, dd, J=3.5, 10.8 Hz), 3.82-3.92 (3H, m), 6.84-6.94 (3H, m), 7.16-7.32 (7H, m), 7.61-7.86 (3H, m)., 7.91 (1H, d, J=5.9 Hz), 8.43-8.48 (1H, m)

MS (m/z): 445 (M+1)

(9) 2(S)-3-[4-(6-chloro-2-methoxyacridin-9-yloxy)phenyl]-2-[(2S)-2-hydroxy-3-(phenoxy)propylamino]propan-1-ol (51.6 mg, 29%) as a yellow solid.

IR (KBr): 3413, 1631, 1502, 1471, 1417, 1232 cm⁻¹

NMR (CDCl₃, δ): 1.98 (3H, br), 2.75-2.98 (5H, m), 3.40-3.65 (2H, m), 3.81 (3H, s), 3.97 (3H, m), 6.77 (1H, d, J=8.5 Hz), 6.86-6.98 (3H, m), 7.10 (1H, d, J=8.5 Hz), 7.16 (1H, d, J=2.7 Hz), 7.22-7.30 (2H, m), 7.36 (1H, dd, J=1.9, 9.2 Hz), 7.47 (1H, dd, J=2.7, 9.5 Hz), 7.96 (1H, d, J=9.2 Hz), 8.12 (1H, d, J=9.5 Hz), 8.22 (1H, d, J=1.9 Hz)

MS (m/z): 559 (M+1)

(10) 2(S)-3-[4-(5-bromoisoquinolin-1-yloxy)-phenyl]-2-[(2S)-2-hydroxy-3-(phenoxy)propylamino]propan-1-ol (26.0 mg, 24%) as a Yellow Solid.

IR (KBr): 3419, 1620, 1595, 1585, 1500, 1481, 1358, 1244, 1217 cm⁻¹

MS (m/z): 523, 525 (M+1)

(11). (3S)-3-[(2S)-2-hydroxy-3-(phenoxy)propylamino]-2-methyl-4-[4-(quinolin-4-yloxy)phenyl]butan-2-ol (26.7 mg, 43.1%) as a White Solid.

IR (KBr): 3423, 1595, 1498, 1248, 1211 cm⁻¹

NMR (CDCl₃, δ): 1.23 (3H, s), 1.32 (3H, s), 1.73 (3H, br), 2.44-3.11 (5H, r), 3.83-3.93 (9H, i), 6.53 (1H, d, J=5.2 Hz), 6.87 (2H, d, J=7.8 Hz), 6.96 (1H, t-like, J=7.4 Hz), 7.15 (2H, d, J=8.4 Hz), 7.23-7.31 (2H, m), 7.36 (2H, d, J=8.4 Hz), 7.55-7.62 (1H, m), 7.73-7.80 (1H, m), 8.10 (1H, d, J=8.4 Hz), 8.37 (1H, d, J=8.3 Hz), 8.61 (1H, d, J=5.2 Hz)

MS (m/z): 473 (M+1)

(12) (3S)-4-[4-(3-carbamoylpyridin-2-yloxy)phenyl]-3-[(2S)-2-hydroxy-3-(phenoxy)propylamino]-2-(methyl)butan-2-ol (109 mg, 67%) as a White Solid.

IR (KBr): 3469, 1670 (C═O), 1587, 1419, 1242 cm⁻¹

NMR (CDCl₃, δ): 1.24 (3H, s), 1.30 (3H, s), 1.73 (3H, br), 2.37-12.61 (4H, m), 3.04-3.11 (1H, m), 3.50 (1H, m), 3.73-3.76 (2H, m), 6.00 (1H, br), 6.73 (2H, d, J=7.8 Hz), 6.93 (1H, t-like, J=7.4 Hz), 7.10 (2H, d, J=8.5 Hz), 7.19-7.26 (2H, m), 7.35 (2H, d, J=8.5 Hz), 7.76 (1H, br), 8.13 (1H, dd, J=2.0, 4.9 Hz), 8.63 (1H, dd, J=2.0, 7.6 Hz)

MS (m/z): 466 (M+1)

(13) (2S)-1-[(1S)-2-methoxy-1-[4-(quinolin-4-yloxy)benzyl]eth ylamino]-3-phenoxypropan-2-ol (46.5 mg, 47%) as a yellow solid.

IR (KBr): 3408, 1593, 1579, 1510, 1311, 1244, 1123 cm⁻¹

NMR (CDCl₃, δ): 2.48-2.77 (2H, m), 2.93-3.00 (1H, m), 3.20 (2H, d, J=5.3 Hz), 3.31-3.35 (2H, m), 3.33 (3H, s, OMe), 4.25 (2H, d, J=5.1 Hz), 4.92-4.97 (1H, m), 6.54 (1H, d, J=8.4 Hz), 6.80-6.99 (6H, m), 7.23-7.31 (2H, m), 7.45-7.52 (1H, m), 7.64-7.72 (1H, m), 8.01-8.08 (2H, m), 8.69 (1H, d, J=5.3 Hz)

MS (m/z): 459 (M+1)

### EXAMPLE 33

Under nitrogen, a solution of 2-[4-(2S)-(2-amino-3-hydroxypropyl)phenoxy]nicotinamide dihydrochloride (350 mg), (2S)-1,2-epoxy-3-(2-chlorophenoxy)propane (132 mg) and N,N-diisopropylethylamine (0.17 ml) in ethanol (10 ml) was refluxed for 7 hours. The mixture was evaporated in vacuo. The residue was purified by column chromatography on silica gel (chloroform:methanol=100:1) to give 2-(4-{3-hydroxy-(2S)-2-[(2S)-2-hydroxy-3-(2-chlorophenoxy)propylamino]-propyl}phenoxy)nicotinamide as a colorless powder.

IR (KBr): 3760-3330, 1747, 1698, 1652, 1540, 1511, 1421 cm⁻¹

NMR (DMSO-d₆, δ): 2.60-3.30 (7H, m), 3.80-3.90 (1H, br s), 3.90-4.00 (2H, m), 4.50-4.60 (1H, m), 4.50-4.60 (1H, m), 5.05-5.10 (1H, br s), 6.80-7.40 (9H, m), 7.70-7.85 (2H, br s), 8.10-8.15 (2H, m)

MS (m/z): 472 (M+1)

### EXAMPLE 34

The following compound was synthesized according to a similar manner to that of Example 33.

2-(4-{(2S)-2-7[(2S)-3-(3-chlorophenoxy)-2-hydroxy-propylamino]-3-hydroxypropyl}phenoxy)nicotinic acid ethyl ester (450 mg) as a Colorless Form

NMR (CDCl₃, δ): 1.40 (3H, t, J=7 Hz), 2.60-2.90 (5H, m), 3.45 (1H, dd, J=4.5, 11 Hz), 3.70 (1H, dd, J=3.5, 11 Hz), 3.80-3.90 (3H, m), 4.40 (2H, t, J=7 Hz), 6.70-7.27 (9H, m), 8.10-8.28 (2H, m)

MALDI-MS (m/z): 501 (M+1)

### EXAMPLE 35

To a solution of 2-(4-{(2S)-2-[(2S)-3-(3-chlorophenoxy)-2-hydroxypropylamino]-3-hydroxypropyl)phenoxy)nicotinic acid ethyl ester (32.3 mg) in methanol (1.0 ml) was added with aqueous 1N sodium hydroxide (0.064 ml), and the mixture was stirred at room temperature for 2.5 hours. The mixture was evaporated in vacuo and the residue was triturated with diisopropyl ether to give sodium 2-(4-((2S)-2-[(2S)-3-(3-chlorophenoxy)-2-hydroxypropylamino]-3-hydroxypropyl}-phenoxy)nicotinic acid (0.52 g) as a colorless powder.

IR (KBr): 3360-3380, 1690, 1619, 1380 cm⁻¹

NMR (D₂O, δ): 2.60-3.00 (5H, m), 3.45-3.60 (2H, m), 3.80-4.10 (3H, m), 6.80-6.92 (1H, m), 7.00-7.09 (4H, m), 7.10-7.40 (4H, m), 7.90-8.05 (2H, m)

MS (m/z): 473 (M+1)

### EXAMPLE 36

Under nitrogen, a solution of (2R)-2-amino-3-[4-(3-cyanopyridin-2-yloxy)phenyl]propanol dihydrochloride (4.9 g), (2S)-3-phenoxy-1,2-epoxypropane (5.0 g) and N,N-diisopropylethylamine (4.5 ml) in a mixture of methanol (10 ml) and 1,4-dioxane (10 ml) was refluxed for 28 hours. The mixture was evaporated in vacuo. The residue was purified by column chromatography on silica gel (dichloromethane:methanol=20:1), followed by treatment with 4N hydrogen chloride in dioxane to give (2R)-3-[4-(3-cyanopyridin-2-yloxy)phenyl]-2-((2S)-2-hydroxy-3-phenoxypropylamino)propanol hydrochloride (1.5 g).

IR (KBr): 3560-3330, 2240, 1648, 1592, 1492 cm⁻¹

NMR (DMSO-d₆, δ): 2.90-3.00 (1H, m), 3.05-3.80. (6H, m), 4.00 (1H, d, J=5.0 Hz), 4.20-4.30 (1H., m), 5.48 (1H, br s), 5.90 (1H, d, J=5.0 Hz), 6.90-7.05 (3H, m), 7.10-7.50 (7H, m), 8.30-8.45 (2H, m), 8.70-8.80 (1H, br s)

MS (m/z): 420 (M+1)

### EXAMPLE 37

To a solution of (R,S)-{2-[3-(3-cyanopyridin-2-yloxy)-phenyl]-1-hydroxymethylethyl}carbamic acid tert-butyl ester (500 mg) in dioxane (10 ml) was added 4N hydrogen chloride in dioxane (10 ml) at room temperature, and the solution was stirred at the same temperature for 3 hours. The mixture was evaporated in vacuo, and the residue was triturated with ethyl acetate to give (R,S)-2-amino-3-[3-(3-cyanopyridin-2-yloxy)phenyl]propanol dihydrochloride (415 mg). Under nitrogen, a solution of (R,S)-2-amino-3-[3-(3-cyanopyridin-2-yloxy)phenyl]propanol dihydrochloride (415 mg), (2S)-3-phenoxy-1,2-epoxypropane (192 mg) and N,N-diisopropyl-ethylamine (0.43 ml) in ethanol (10 ml) was refluxed for 7 hours. The mixture was evaporated in vacuo. The residue was purified by column chromatography on silica gel (chloroform:methanol=100:1), followed by treatment with 4N hydrogen chloride in dioxane to give (R,S)-3-[3-(3-cyanopyridin-2-yloxy)phenyl]-2-(2-hydroxy-3-phenoxypropylamino)propanol hydrochloride (174.7 mg) as a colorless powder.

IR (KBr): 3360-3330, 2227, 1690, 1590, 1425, 1240 cm⁻¹

NMR (DMSO-d₆, δ): 2.80-3.80 (7H, m), 4.00 (1H, d, J=5.2 Hz), 4.20-4.30 (1H, m), 5.44 (1H, br s), 6.90-7.00 (3H, m), 7.10-7.45 (7H, m), 8.30-8.46 (2H, m), 8.70-8.80 (1H, br s)

MS (m/z): 420 (M+1)

### EXAMPLE 38

Under nitrogen, a solution of (2S)-2-amino-3-[4-(3-nitropyridin-2-yloxy)phenyl]propanol hydrochloride (3.1 g), (2S)-1,2-epoxy-3-phenoxypropane (1.43 g) and N,N-diisopropylethylamine (3.2 ml) in ethanol (30 ml) was refluxed for 4 hours. The mixture was evaporated in vacuo. The residue was purified by column chromatography on silica gel (dichloromethane:methanol=25:1), followed by treatment with 4N hydrogen chloride in ethyl acetate and trituration with ethyl acetate to give (2S)-2-((2S)-2-hydroxy-3-phenoxypropylamino)-3-[4-(3-nitropyridin-2-yloxy)phenyl]-propanol (0.96 g).

MS (m/z): 440 (M+1)

### EXAMPLE 39

To a solution of (2S)-2-((2S)-2-hydroxy-3-phenoxypropylamino)-3-[4-(3-nitropyridin-2-yloxy)phenyl]propanol (100 mg) in dioxane (3 ml) was added 4N hydrogen chloride in dioxane (3 ml) at room temperature, and the solution was stirred at the same temperature for 3 hours. The mixture was evaporated in vacuo, and the residue was triturated with diisopropyl ether to give (2S)-2-((2S)-2-hydroxy-3-phenoxypropylamino)-3-[4-(3-nitropyridin-2-yloxy)phenyl]propanol hydrochloride (80 mg).

NMR (DMSO-d₆, δ): 2.70-3.70 (7H, m), 4.00-4.10 (2H, m), 4.20-4.30 (1H, m), 5.50 (1H, br s), 5.80 (1H, br s), 6.90-7.00 (6H, m), 7.10-7.40 (8H, m), 8.50-8.70 (2H, m)

MS (m/z): 440 (M+1)

### EXAMPLE 40

A mixture of {(1S)-1-hydroxymethyl-2-[4-(3-nitropyridin-2-yloxy)phenyl]ethyl}-(2S)-(2-hydroxy-3-phenoxypropyl)-carbamic acid tert-butyl ester (100 mg), 10% palladium on activated carbon (50% wet, 20 mg) and methanol (2.0 ml) was stirred at room temperature in the presence of hydrogen at an atmospheric pressure for 1 hour, and filtered. The filtrate was evaporated in vacuo. To a solution of the residue in dioxane (3 ml) was added 4N hydrogen chloride in dioxane (3 ml) at room temperature, and the solution was stirred at the same temperature for 3 hours. The mixture was evaporated in vacuo, and the residue was triturated with diisopropyl ether to give 3-[4-(3-aminopyridin-2-yloxy)phenyl]-(2S)-2-((2S)-2-hydroxy-3-phenoxypropylamino)propanol dihydrochloride (85 mg) as a pale yellow powder.

NMR (DMSO-d₆, δ): 2.80-2.90 (1H, m), 3.10-3.80 (6H, m); 3.90-4.00 (2H, m), 4.25-4.30 (1H, m), 5.10-5.20 (3H, br s), 6.90-7.10 (6H, m), 7.20-7.40 (5H, m), 7.60-7.70 (1H, m), 8.55-8.75 (1H, br s), 9.23-9.50 (1H, br s)

MS (m/z) 410 (M+1)

### EXAMPLE 41

Under nitrogen, a solution of 2-[4-((2S)-2-amino-3-hydroxypropyl)phenoxy]nicotinamide dihydrochloride (400 mg), (2S)-1,2-epoxy-3-(4-benzyloxyphenoxy)propane (284 mg) and N,N-diisopropylethylamine (0.57 ml) in ethanol (10 ml) was refluxed for 7 hours. The mixture was evaporated in vacuo. The residue was purified by column chromatography on silica gel (chloroform:methanol=10011) to give 2-(4-{3-hydroxy-(2S)-2-[(2S)-2-hydroxy-3-(4-benzyloxyphenoxy)propylamino]-propyl}phenoxy)nicotinamide (200 mg) as a colorless form.

MS (m/z): 544 (M+1)

### EXAMPLE 42

A mixture of 2-(4-{3-hydroxy-(2S)-2-[(2S)-2-hydroxy-3-(4-benzyloxyphenoxy)propylamino]propyl}phenoxy)nicotinamide (150 mg), 10% palladium on activated carbon (50% wet, 50 mg) and methanol (10.0 ml) was stirred at room temperature in the presence of hydrogen at an atmospheric pressure for 1 hour, and filtered. The filtrate was evaporated in vacuo. The residue was chromatographed (chloroform-methanol) over silica gel to afford 2-(4-{3-hydroxy-(2S)-2-[(2S)-2-hydroxy-3-(4-hydroxyphenoxy)propylamino]propyl}phenoxy)nicotinamide. To a solution of 2-(4-{3-hydroxy-(2S)-2-[(2S)-2-hydroxy-3-(4-hydroxyphenoxy)propylamino]propyl}phenoxy)nicotinamide in dioxane (20 ml) was added 4N hydrogen chloride in dioxane (10 ml) at room temperature, and the solution was stirred at the same temperature for 3 hours. The mixture was evaporated in vacuo, and the residue was triturated with diisopropyl ether to give 2-(4-{3-hydroxy-(2S)-2-[(2S)-2-hydroxy-3-(4-hydroxyphenoxy)propylamino]propyl)phenoxy)nicotinamide dihydrochloride (7.69 g) as a colorless powder.

IR (KBr): 3760-3330, 1747, 1698, 1650, 1540, 1513, 1456 cm⁻¹

NMR (DMSO-d₆, δ): 2.70-2.95 (1H, m), 3.10-3.90 (8H, m), 4.20-4.30 (1H, m), 6.67-6.82 (4H, m), 7.10-7.40 (5H, m), 7.78 (1H, br s), 8.10-8.20 (2H, m), 8.50 (1H, br s), 9.10 (1H, br s)

MS (m/z): 454 (M+1)

### EXAMPLE 43

Under nitrogen, to a solution of (S)-2-amino-3-[4-(3-hydroxymethylpyridin-2-yloxy)phenyl]propan-1-ol dihydrochloride (200 mg) in methanol (5 ml) were added N,N-diisopropylethylamine (0.25 ml) and (S)-N-(2-benzyloxy-5-oxiranylmethoxyphenyl)methanesulfonamide (199 mg) at room temperature, and the mixture was refluxed for 19 hours. After removal of the solvent in vacuo, the residue was purified by column chromatography on silica gel (chloroform:methanol: 28% ammonium hydroxide in water=80:8:1 to 20:2:1) to give N-[2-benzyloxy-5-((2S)-2-hydroxy-3-{(1S)-1-hydroxymethyl-2-[4-(3-hydroxymethylpyridin-2-yloxy)-phenyl]ethylamino}propoxy)phenyl]methanesulfonamide (22 mg).

NMR (CDCl₃, δ): 2.6-2.9 (8H, m), 3.3-4.1 (5H, m), 4.79 (2H, s), 5.02 (2H, s), 6.5-6.75 (2H, m), 6.85-7.45 (11H, m), 7.75-7.90 (1H, m), 7.95-8.00 (1H, m)

### EXAMPLE 44

A mixture of N-[2-benzyloxy-5-((2S)-2-hydroxy-3-{(1S)-1-hydroxymethyl-2-[4-(3-hydroxymethylpyridin-2-yloxy)phenyl]ethylaminopropoxy}phenyl]methanesulfonamide (20 mg) and 10% palladium on activated carbon (50% wet, 10 mg) in methanol (3 ml) was stirred at room temperature in the presence of hydrogen at an atmospheric pressure for 2 hours. After filtration, the filtrate was evaporated in vacuo, followed by treatment with 4N hydrogen chloride in 1,4-dioxane and trituration with hexane to give N-[2-hydroxy-5-((2S)-2-hydroxy-3-{(1S)-1-hydroxymethyl-2-[4-(3-hydroxymethylpyridin-2-yloxy)phenyl]ethylamino}propoxy)-phenyl]methanesulfonamide dihydrochloride (6 mg).

NMR (DMSO-d₆, δ): 2.3-4.0 (12H, m), 4.1-4.3 (1H, m), 4.62 (1H, s), 6.6-7.4 (8H, m), 7.8-8.0 (2H, m)

### EXAMPLE 45

Under nitrogen, to a solution of (S)-2-amino-3-[4-(3-hydroxymethylpyridin-2-yloxy)phenyl]propan-1-ol dihydrochloride (300 mg) in methanol (15 ml) were added N,N-diisopropylethylamine (0.38 ml) and (S)-[5-(oxiranyl)-methoxypyridin-2-yl]carbamic acid benzyl ester (260 mg) at room temperature, and the mixture was refluxed for 20 hours. After removal of the solvent in vacuo, the residue was dissolved in a mixture of saturated aqueous sodium hydrogencarbonate and ethyl acetate. After separation, the organic layer was washed with brine, dried over anhydrous magnesium sulfate, and evaporated in vacuo. The residue was purified by column chromatography on silica gel (chloroform:methanol=5:1) to give [5-((2S)-2-hydroxy-3-{(2S)-1-hydroxymethyl-2-[4-(3-hydroxymethylpyridin-2-yloxy)-phenyl]ethylamino}propoxy)pyridin-2-yl]carbamic acid benzyl ester (56 mg).

NMR (CDCl₃, δ): 2.6-2.95 (5H, m), 3.4-3.9 (5H, m), 4.84 (2H, s), 5.21 (2H, s), 6.9-7.45 (10H, m), 7.57 (1H s), 7.7-7.8 (2H, m), 7.87 (1H, d, J=9.1 Hz), 7.9-8.1 (1H, m)

### EXAMPLE 46

The following compounds were obtained according to a similar manner to that of Example 44.

(1) (2S)-2-[(2S)-3-(6-Aminopyridin-3-yloxy)-2-hydroxypropylamino]-3-[4-(3-hydroxymethylpyridin-2-yloxy)phenyl]propan-1-ol tetrahydrochloride (26 mg)

NMR (DMSO d₆, δ) 2.8-3.75 (7H, m) 3.95-4.05 (2H, m), 4.15-4.3 (1H, m), 4.61 (2H, s), 7.0-7.2 (4H, m), 7.33 (2H, d, J=8.5 Hz), 7.68 (1H, d, J=2.6 Hz), 7.7-8.0 (3H, m)

(2) N-(2-Hydroxy-5-{(1R)-1-hydroxy-2-[(1S)-1-hydroxymethyl-2-(4-phenoxyphenyl)ethylamino]ethyl}phenyl)-methanesulfonamide hydrochloride (70 mg)

NMR (DMSO-d₆, δ): 2.7-4.1 (10H, m), 4.6-4.9 (1H, m), 6.9-7.5 (12H, m)

(3) N-[5-((1R)-2-{(1S)-2-[4-(4-Chlorophenoxy)phenyl]-1-hydroxymethylethylamino}-1-hydroxyethyl)-2-hydroxyphenyl]methanesulfonamide hydrochloride (130 mg).

NMR (DMSO₆, δ): 2.8-4.1 (10H, m), 4.55-4.9 (1H, m), 6.9-7.5 (11H, m)

(4) N-[2-Hydroxy-5-((1R)-1-hydroxy-2-{(1S)-1-hydroxymethyl-2-[4-(naphthalen-1-yloxy)phenyl]ethylamino}ethyl)-phenyl]methanesulfonamide hydrochloride (53 mg)

NMR (DMSO-d₆, δ): 2.7-4.1 (10H, m), 4.55-4.9 (1H, m), 6.9-7.7 (11H, m), 7.76 (1H, d, J=8.2 Hz), 7.9-8.15 (2H, m)

### EXAMPLE 47

The following compounds were obtained according to a similar manner to that of Example 45.

(1) 2-{4-[(2S)-3-Hydroxy-2-((2S)-2-hydroxy-3-phenoxypropyl-amino)propyl]phenoxy}nicotinic acid ethyl ester (90 mg)

NMR (CDCl₃, δ): 1.40 (3H, t, J=7.1 Hz), 2.65-2.95 (5H, m), 3.4-3.5 (1H, m), 3.65-3.75 (1H, m), 3.8-4.0 (3H, m), 4.42 (2H, q, J=7.1 Hz), 6.8-7.35 (12H, m), 8.15-8.3 (2H, m)

(2) 4-{4-[(2S)-3-Hydroxy-2-((2S)-2-hydroxy-3-phenoxypropyl-amino)propyl]phenoxy}pyridine-2-carboxylic acid amide (21 mg)

NMR (DMSO-d₆, δ): 2.5-2.9 (4H, m), 3.25-3.5 (3H, m), 3.7-4.0 (3H, m), 6.85-7.45 (11H, m), 8.51 (1H, d, J=5.6 Hz)

(3) 4-(4-{(2S)-2-[(2S)-3-(3-Chlorophenoxy)-2-hydroxypropyl-amino]-3-hydroxypropyl}phenoxy)pyridine-2-carboxylic acid amide (97 mg)

NMR (DMSO-d₆, δ): 2.55-2.8 (5H, m), 3.15-3.5 (2H, m), 3.75-4.0 (3H, m), 7.85-7.4 (10H, m), 8.50 (1H, d, J=5.6 Hz)

(4) (2S)-2-((2S)-2-Hydroxy-3-phenoxypropylamino)-3-(4-phenoxyphenyl)propan-1-ol (80 mg)

NMR (DMSO-d₆, δ): 2.5-2.8 (5H, m), 3.2-3.45 (2H, m), 3.75-3.95 (3H, m), 6.85-7.0 (7H, m), 7.1-7.45 (7H, m)

(5) (2S)-3-[4-(4-Chlorophenoxy)phenyl]-2-((2S)-2-hydroxy-3-phenoxypropylamino)propan-1-ol hydrochloride (110 mg)

NMR (DMSO-d₆, δ): 2.5-2.85 (5H, m), 3.15-3.45 (2H, m), 3.75-3.95 (2H, m), 4.5-4.65 (1H, m), 6.85-7.0 (7H, m), 7.15-7.45 (6H, m)

(6) (2S)-2-((2S)-2-Hydroxy-3-phenoxypropylamino)-3-[4-(quinolin-2-yloxy)phenyl]propan-1-ol (60 mg)

NMR (DMSO-d₆, δ): 2.5-2.9 (5H, m), 3.2-3.5 (2H, m), 3.8-4.0 (3H, m), 6.85-7.0 (3H, m), 7.1-7.35 (7H, m), 7.45-7.55 (1H, m), 7.6-7.65 (2H, m), 7.94 (1H, d, J=7.9 Hz), 8.39 (1H, d, J=8.8 Hz)

(7) (2S)-2-((2S)-2-Hydroxy-3-phenoxypropylamino)-3-(4-phenylsulfanylphenyl)propan-1-ol (110 mg)

NMR (DMSO-d₆, δ): 2.5-2.8 (5H, m), 3.15-3.4 (2H, m), 3.75-3.95 (3H, m), 6.85-7.0 (3H, m), 7.2-7.4 (11H, m)

### EXAMPLE 48

To a solution of 2-{4-[(2S)-3-hydroxy-2-((2S)-2-hydroxy-3-phenoxypropylamino)propyl]phenoxy}nicotinic acid ethyl ester (40 mg) in ethanol (3 ml) was added aqueous 1N sodium hydroxide (86 μl) at 5° C., and the mixture was stirred at room temperature for 12 hours. After evaporation in vacuo, the residue was triturated with hexane and dried in vacuo to give sodium 2-{4-[(2S)-3-hydroxy-2-((2S)-2-hydroxy-3-phenoxypropylamino)propyl]phenoxy}nicotinate (32 mg).

NMR (D₂O, δ): 2.6-3.1 (5H, m), 3.5-3.75 (2H, m), 3.9-4.2 (3H, m), 6.9-7.5 (10H, m), 7.9-8.1 (2H, m)

### EXAMPLE 49

Under nitrogen, to a solution of (S)-2-amino-3-[4-(pyrimidin-2-yloxy)phenyl]propan-1-ol dihydrochloride (230 mg) in ethanol (5 ml) were added N,N-diisopropylethylamine (0.62 ml) and (S)-3-phenoxy-1,2-epoxypropane (110 mg) at room temperature, and the mixture was refluxed for 6 hours. After removal of the solvent in vacuo, the residue was dissolved in a mixture of saturated aqueous sodium hydrogencarbonate and ethyl acetate. After separation, the organic layer was washed with brine, dried over anhydrous magnesium sulfate, and evaporated in vacuo. The residue was purified by column chromatography on silica gel (chloroform:methanol=10:1 to 5:1), followed by treatment with 4N hydrogen chloride in 1,4-dioxane, trituration with hexane and dryness in vacuo to give (2S)-2-((2S)-2-hydroxy-3-phenoxypropylamino)-3-[4-(pyrimidin-2-yloxy)phenyl]propan-1-ol dihydrochloride (65 mg).

NMR (DMSO-d₆, δ): 2.85-3.0 (1H, m), 3.1-3.75 (6H, m), 3.9-4.05 (2H, m), 4.2-4.35 (1H, m), 6.9-7.05 (3H, m), 7.1-7.45 (7H, m), 8.64 (2H, d, J=4.8 Hz)

### EXAMPLE 50

The following compounds were obtained according to a similar manner to that of Example 49.

(1) (2S)-2-((2S)-2-Hydroxy-3-phenoxypropylamino)-3-[4-(pyrazin-2-yloxy)phenyl]propan-1-ol dihydrochloride (36 mg)

NMR (DMSO-d₆, δ): 2.8-3.0 (1H, m), 3.05-3.7 (4H, m), 3.95-4.05 (2H, m), 4.150-4.35 (1H, m), 6.9-7.0 (3H, m), 7.19 (2H, d, J=8.5 Hz), 7.25-7.45 (4H, m); 8.19-8.21 (1H, m), 8.38 (1H, d, J=2.7 Hz), 8.53-8.54 (1H, m)

(2) 2-{4-[(2S)-3-Hydroxy-2-((2S)-2-hydroxy-3-phenoxy-propylamino)propyl]phenoxy}isonicotinamide dihydrochloride (30 mg)

NMR (DMSO-d₆, δ): 2.8-3.8 (7H, m), 3.95-4.1 (2H, m), 4.15-4.3 (1H, m), 6.9-7.05 (3H, m), 7.13 (2H, d, J=8.4 Hz), 7.25-7.4 (5H, m), 7.5-7.55 (1H, m), 8.26 (1H, d, J=5.2 Hz)

(3) 6-{4-[(2S)-3-Hydroxy-2-((2S)-2-hydroxy-3-phenoxy-propylamino)propyl]phenoxy}nicotinamide dihydrochloride (17 mg)

NMR (DMSO-d₆, δ): 2.9-3.9 (7H, m), 4.0-4.35 (3H, m), 6.9-7.25 (6H, m), 7.25-7.5 (4H, m), 8.2-8.35 (1H, m), 8.55-8.65 (1H, m)

(4) 6-{4-[(2S)-3-Hydroxy-2-((2S)-2-hydroxy-3-phenoxy-propylamino)propyl]phenoxy}pyridine-2-carboxylic acid amide hydrochloride (7 mg)

NMR (CD₃OD, δ): 2.95-4.2 (9H, m), 4.25-4.4 (1H, m), 6.9-8.2 (12H, m)

(5) 2-(4-{(2S)-2-[(2S)-3-(3-Chlorophenoxy)-2-hydroxypropylamino]-3-hydroxypropyl}phenoxy)isonicotinamide dihydrochloride (100 mg)

NMR (DMSO-d₆, δ): 2.8-3.75 (7H, m), 4.0-4.1 (2H, m), 4.15-4.3 (1H, m), 6.95-7.2 (5H, m), 7.3-7.4 (4H, m), 7.5-7.55 (1H, m), 8.26 (1H, d, J=5.2 Hz)

(6) 6-(4-{(2S)-2-[(2S)-3-(3-Chlorophenoxy)-2-hydroxy-propylamino]-3-hydroxypropyl}phenoxy)nicotinamide dihydrochloride (30 mg)

NMR (DMSO-d₆, δ): 2.8-3.8 (7H, m), 4.0-4.1 (2H, m), 4.15-4.3 (1H, m), 6.9-7.2 (7H, m), 7.3-7.45 (3H, m), 8.25-8.35 (1H, m), 8.16 (1H, d, J=2.4 Hz)

(7) 6-(4-{(2S)-2-[(2S)-3-(3-Chlorophenoxy)-2-hydroxy-propylamino]-3-hydroxypropyl}phenoxy)pyridine-2-carboxylic acid amide hydrochloride (35 mg)

NMR (DMSO-d₆, δ): 2.8-3.75 (7H, m), 4.0-4.1 (2H, m), 4.15-4.35 (1H, m), 6.9-7.5 (10H, m), 7.77 (1H, d, J=7.3 Hz), 8.02 (1H, d, J=7.7 Hz)

(8) 2-(4-{(2S)-2-[(2S)-3-(3-Chlorophenoxy)-2-hydroxypropylamino]-3-hydroxypropyl}phenoxy)-N-methylnicotinamide hydrochloride (70 mg)

NMR (DMSO-d₆, δ): 2.75-3.75 (10H, m), 4.0-4.1 (2H, m), 4.15-4.3 (1H, m), 6.9-7.4 (9H, m), 8.1-8.2 (2H, m)

(9) (2S)-2-((2S)-2-Hydroxy-3-phenoxypropylamino)-3-[4-(naphthalen-1-yloxy)phenyl]propan-1-ol hydrochloride (140 mg)

NMR (DMSO-d₆, δ): 2.75-3.75 (7H, m), 3.9-4.05 (2H, m), 4.15-4.35 (1H, m), 6.85-7.1 (6H, m), 7.25-7.65 (7H, m), 7.74 (1H, d, J=8.3 Hz), 7.95-8.15 (2H, m)

### EXAMPLE 51

Under nitrogen, absolution of (S)-2-amino-3-(4-phenoxyphenyl)propan-1-ol hydrochloride (300 mg), (R)-N-{2-benzyloxy-5-[2-iodo-1-(triethylsilyloxy)etyhyl]phenyl}-methanesulfonamide (600 mg) and N,N-diisopropylethylamine (0.75 ml) in 1,3-dimethyl-2-imidazolidinone (5 ml) was stirred at 120° C. for 60 hours. The resulting mixture was poured into saturated aqueous sodium hydrogencarbonate and the aqueous mixture was extracted with ethyl acetate. The organic layer was washed with brine, dried over anhydrous magnesium sulfate, and evaporated in vacuo. To a mixture of the residue in 1,4-dioxane (4 ml) was added 4N hydrogen chloride in 1,4-dioxane (1 ml), and the mixture was stirred at room temperature for 1.5 hours. After evaporation in vacuo, the residue was dissolved in a mixture of saturated aqueous sodium hydrogencarbonate and ethyl acetate, followed by separation. The organic layer was washed with brine, dried over anhydrous magnesium sulfate, and evaporated in vacuo. The residue was purified by column chromatography on silica gel (chloroform:methanol=20:1 to 10:1) to give N-(2-benzyloxy-5-{(1R)-1-hydroxy-2-[(1S)-1-hydroxymethyl-2-(4-phenoxyphenyl)ethylamino]ethyl}phenyl)methanesulfonamide (170 mg).

NMR (CDCl₃, δ): 2.6-3.1 (8H, m), 3.35-3.5 (1H, m), 3.55-3.7 (1H, m), 4.55-4.7 (1H, m), 5.09 (2H, s), 6.9-7.2 (9H, m), 7.25-7.5 (8H, m)

### EXAMPLE 52

The following compounds were obtained according to a similar manner to that of Example 51.

(1) N-[2-Benzyloxy-5-((1R)-2-{(1S)-2-[4-(4-chlorophenoxy)-phenyl]-1-hydroxymethylethylamino}-1-hydroxyethyl)phenyl]methanesulfonamide (160 mg)

NMR (DMSO-d₆, δ): 2.45-2.8 (5H, m), 2.88 (3H, s), 3.15-3.45 (2H, m), 4.45-4.55 (1H, m), 5.14 (2H, s), 6.85-7.6 (16H, m)

(2) N-[2-Benzyloxy-5-((1R)-1-hydroxy-2-{(1S)-1-hydroxymethyl-2-[4-(naphthalen-1-yloxy)phenyl]ethylamino}-ethyl)phenyl]methanesulfonamide (99 mg)

NMR (DMSO-d₆, δ): 2.5-2.8 (5H, m), 2.89 (3H, s), 3.2-3.5 (2H, m), 4.5-4.6 (1H, m), 5.13 (2H, s), 6.9-7.85 (17H, m), 7.95-8.15 (2H, m)

(3) N-[2-Benzyloxy-5-((1R)-1-hydroxy-2-{(1S)-1-hydroxymethyl-2-[4-(quinolin-2-yloxy)phenyl]ethylamino}ethyl)-phenyl]methanesulfonamide (28 mg)

NMR (DMSO-d₆, δ): 2.5-2.95 (8H, m), 3.15-3.55 (2H, m), 4.45-4.6 (1H, m), 5.13 (2H, s), 7.0-7.75 (14H, m), 7.85-8.0 (2H, m), 8.3-8.45 (2H, m)

(4) N-[2-Benzyloxy-5-((1R)-1-hydroxy-2-{(1S)-1-hydroxymethyl-2-[4-(quinolin-3-yloxy)phenyl]ethylamino)ethyl)-phenyl]methanesulfonamide (23 mg)

NMR (CDCl₃, δ): 2.7-3.05 (8H, m), 3.4-3.5 (1H, m), 3.65-3.75 (1H, m), 4.55-4.7 (1H, m), 5.09 (2H, s), 6.95-7.7 (16H, m), 8.14 (1H, d, J=9.5 Hz), 8.78 (1H, d, J=2.8 Hz)

(5) N-(2-Benzyloxy-5-{(1R)-1-hydroxy-2-[(1S)-1-hydroxymethyl-2-(4-phenylsulfanylphenyl)ethylamino]ethyl}-phenyl)methanesulfonamide (96 mg)

NMR (CDCl₃, δ): 2.65-3.05 (8H, m), 3.3-3.5 (1H, m), 3.55-3.7 (1H, m), 4.55-4.7 (1H, m), 5.10 (2H, s), 6.97 (1H, d, J=8.4 Hz), 7.1-7.5 (16H, m)

### EXAMPLE 53

A mixture of N-[2-benzyloxy-5-((1R)-1-hydroxy-2-{(1S)-1-hydroxymethyl-2-[4-(quinolin-2-yloxy)phenyl]ethylamino}-ethyl)phenyl]methanesulfonamide (25 mg) and 10% palladium on activated carbon (50% wet, 10 mg) in methanol (3 ml) was stirred at room temperature in the presence of hydrogen at an atmospheric pressure for 3.5 hours. After filtration, the filtrate was evaporated in vacuo, and the residue was purified by preparative thin layer chromatography (silica gel, chloroform:methanol=5:1) to give N-[2-hydroxy-5-((1R)-1-hydroxy-2-{(1S)-1-hydroxymethyl-2-[4-(quinolin-2-yloxy)-phenyl]ethylamino}ethyl)phenyl]methanesulfonamide (7 mg).

NMR (DMSO-d₆, δ): 2.5-2.95 (5H, m), 2.93 (3H, s), 3.15-3.6 (2H, m), 4.45-4.6 (1H, m), 6.85 (1H, d, J=8.2 Hz), 6.95-7.35 (7H, m), 7.45-7.55 (1, m), 7.6-7.75 (2H, m), 7.94 (1H, d, J=7.8 Hz), 8.39 (1H, d, J=8.8 Hz)

### EXAMPLE 54

Under nitrogen, to a stirred solution of (S)-2-amino-3-[4-(quinolin-3-yloxy)phenyl]propan-1-ol dihydrochloride (200 mg) in ethanol (5 ml) was added sodium methoxide (28% in methanol, 0.21 ml) at 5° C. After 15 minutes, a solution of (S)-3-phenoxy-1,2-epoxypropane (82 mg) in ethanol (1 ml) was added and the mixture was refluxed for 7.5 hours. The reaction mixture was evaporated in vacuo. The residue was purified by column chromatography on silica gel (chloroform:methanol=20:1 to 10:1) to give (2S)-2-((2S)-2-hydroxy-3-phenoxypropylamino)-3-[4-(quinolin-3-yloxy)phenyl]-propan-1-ol (110 mg).

NMR (DMSO-d₆, δ): 2.55-2.85 (5H, m), 3.2-3.5 (2H, m), 3.75-4.0 (3H, m), 6.85-6.95 (3H, m), 7.05 (2H, d, J=8.4 Hz), 7.2-7.35 (4H, m), 7.5-7.75 (3H, m), 7.85-7.9 (1H, m), 8.02 (1H, d, J=8.3 Hz), 8.79 (1H, d, J=2.8 Hz)

### EXAMPLE 55

A mixture of N-[2-benzyloxy-5-((1R)-1-hydroxy-2-{(1S)-1-hydroxymethyl-2-[4-(quinolin-3-yloxy)phenyl]ethylamino)}-ethyl)phenyl]methanesulfonamide (20 mg) and 10% palladium on activated carbon (50% wet, 10 mg) in methanol (3 ml) was, stirred at room temperature in the presence of hydrogen at an atmospheric pressure for 2 hours. After filtration, the filtrate was evaporated in vacuo and dried in vacuo to give N-[2-hydroxy-5-((1R)-1-hydroxy-2-{(1S)-1-hydroxymethyl-2-[4-(quinolin-3-yloxy)phenyl]ethylamino}ethyl)phenyl]methanesulfonamide (11 mg).

NMR (DMSO-d₆, δ): 2.6-3.85 (10H, m), 4.85-4.9 (1H, m), 6.85-7.3 (6H, m), 7.36 (2H, d, J=8.5 Hz), 7.55-7.75 (2H, m), 7.80 (1H, d, J=2.7 Hz), 7.91 (1H, d, J=7.1 Hz), 8.04 (1H, d, J=8.2 Hz), 8.78 (1H, d, J=2.8 Hz)

### EXAMPLE 56

The following compound was obtained according to a similar manner to that of Preparation 50.

4-(4-{(2S)-2-[Benzyl-((2S)-2-hydroxy-3-phenoxypropyl)-amino]-3-hydroxypropyl}phenoxy)quinoline-7-carboxylic acid ethyl ester (39 mg)

NMR (CDCl₃, δ): 1.46 (3H, t, J=7.1 Hz), 2.6-3.3 (5H, m), 3.55-4.0 (7H, m), 4.48 (2H, q, J=7.1 Hz), 6.59 (1H, d, J=5.2 Hz), 6.84 (2H, d, J=7.8 Hz), 6.96 (1H, t, J=7.4 Hz), 7.11 (2H, d, J=8.5 Hz), 7.2-7.4 (9H, m), 8.18 (1H, ABq, J=1.6, 8.7 Hz), 8.42 (1H, d, J=8.7 Hz), 8.72 (1H, d, J=5.2 Hz), 8.82 (1H, d, J=1.4 Hz)

### EXAMPLE 57

Under nitrogen, a solution of (2S)-2-{benzyl[(2R)-2-(4-benzyloxy-3-nitrophenyl)-2-hydroxyethyl]amino}-3-[4-(pyridin-2-yloxy)phenyl]propan-1-ol (210 mg) and (R)-2-(4-benzyloxy-3-nitrophenyl)oxirane (170 mg) in ethanol (5 ml) was refluxed for 0.15 hours. After removal of the solvent in vacuo, the residue was purified by column chromatography on silica gel (chloroform:ethyl acetate=20:1 to 5:1) to give (2S)-2-{benzyl[(2R)-2-(4-benzyloxy-3-nitrophenyl)-2-hydroxyethyl]-amino}-3-[4-(pyridin-2-yloxy)phenyl]propan-1-ol (190 mg).

NMR (CDCl₃, δ): 2.55-3.0 (4H, m), 3.1-3.3 (1H, m), 3.55-3.95 (4H, m), 4.35-4.5 (1H, m), 5.20 (2H, s), 6.85-7.5 (18H, m), 7.65-7.75 (2H, m), 8.15-8.2 (1H, m)

### EXAMPLE 58

To a solution of (2S)-2-{benzyl[(2R)-2-(4-benzyloxy-3-nitrophenyl)-2-hydroxyethyl]amino}-3-[4-(pyridin-2-yloxy)-phenyl]propan-1-ol (170 mg) in a mixture of ethanol (3 ml) and water (1 ml) were added powdered iron (48 mg) and ammonium chloride (8 mg) at room temperature, and the mixture was refluxed for 80 minutes. Insoluble materials were filtered off. The filtrate was evaporated in vacuo. The residue was dissolved into a mixture of saturated aqueous sodium hydrogencarbonate and ethyl acetate. After separation, the organic layer was dried over anhydrous magnesium sulfate, evaporated in vacuo and dried in vacuo to give (2S)-2-{[(2R)-2-(3-amino-4-benzyloxyphenyl)-2-hydroxyethyl]benzylamino}-3-[4-(pyridin-2-yloxy)phenyl]propan-1-ol (160 mg).

NMR (CDCl₃, δ): 2.5-2.65 (1H, m), 2.7-3.0 (3H, m), 3.05-3.25 (1H, m), 3.5-4.0 (4H, m), 4.4-4.5 (1H, m), 5.06 (2H, s), 6.5-7.5 (19H, m), 7.6-7.7 (1H, m), 8.2-8.25 (1H, m)

### EXAMPLE 59

Under nitrogen, a solution of (2S)-2-{[(2R)-2-(3-amino-4-benzyloxyphenyl)-2-hydroxyethyl]benzylamino}-3-[4-(pyridin-2-yloxy)phenyl]propan-1-ol (76 mg), methanesulfonyl chloride (11 μl) and pyridine (16 μl) in dichloromethane (3 ml) was stirred at 5° C. for 140 minutes. The resulting mixture was poured into water and the aqueous mixture was extracted with ethyl acetate. The organic layer was washed with brine, dried over anhydrous magnesium sulfate and evaporated in vacuo. The residue was purified by column chromatography on silica gel (chloroform:methanol=20:1) to give N-{5-[(1R)-2-(benzyl-{(1S)-1-hydroxymethyl-2-[4-(pyridin-2-yloxy)phenyl]-ethyl}amino)-1-hydroxyethyl]-2-benzyloxyphenyl}-methanesulfonamide (68 mg).

NMR (CDCl₃, δ): 2.5-3.15 (8H, m), 3.4-3.5 (2H, m), 3.7-3.95 (2H, m), 4.52 (1H, d, J=6.6 Hz), 5.09 (2H, s), 6.7-7.5 (19H, m), 7.6-7.7 (1H, m), 8.15-8.2 (1H, m)

### EXAMPLE 60

The following compound was obtained according to a similar manner to that of Example 55.

N-[2-Hydroxy-5-((1R)-1-hydroxy-2-{(1S)-1-hydroxymethyl-2-[4-(pyridin-2-yloxy)phenyl]ethylamino}ethyl)phenyl]-methanesulfonamide (27 mg)

NMR (DMSO-d₆, δ): 2.7-3.7 (10H, m), 4.85-4.95 (1H, m), 6.9-7.4 (9H, m), 7.8-7.9 (1H, m), 8.1-8.2 (1H, m)

### EXAMPLE 61

The following compounds were obtained according to a similar manner to that of Example 53.

(1) 4-{4-[(2S)-3-Hydroxy-2-((2S)-2-hydroxy-3-phenoxypropylamino)propyl]phenoxy}quinoline-7-carboxylic acid ethyl ester (26 mg)

NMR (DMSO-d₆, δ): 1.40 (3H, t, J=7.1 Hz), 2.55-2.9 (5H, m), 3.1-3.5 (2H, m), 3.7-4.0 (3H, m), 4.42 (2H, q, J=7.0 Hz), 6.66 (1H, d, J=5.1 Hz), 6.9-7.0 (3H, m), 7.15-7.45 (6H, m), 8.13 (1H, ABq, J=1.5, 8.7 Hz), 8.44 (1H, d, J=8.7 Hz), 8.60 (1H, m), 8.76 (1H, d, J=5.2 Hz)

(2) 4-{4-[(2S)-3-Hydroxy-2-((2S)-2-hydroxy-3-phenoxypropylamino)propyl]phenoxy}quinoline-7-carboxylic acid amide (15 mg)

NMR (DMSO-d₆, δ): 2.55-2.85 (5H, m), 3.15-3.5 (2H, m), 3.75-4.0 (3H, m), 6.61 (1H, d, J=5.1 Hz), 6.9-7.0 (3H, m), 7.15-7.4 (5H, m), 8.09 (1H, ABq, J=1.6, 8.7 Hz), 8.25-8.4 (2H, m), 8.57 (1H, d, J=1.4 Hz), 8.72 (1H, d, J=5.2 Hz)

### EXAMPLE 62

To a solution of 4-{4-[(2S)-3-hydroxy-2-((2S)-2-hydroxy-3-phenoxypropylamino)propyl]phenoxy}quinoline-7-carboxylic acid ethyl ester (21 mg) in ethanol (3 ml) was added 1N aqueous sodium hydroxide (41 μM) at room temperature, and the mixture was stirred at the same temperature for 1 hour. The reaction mixture was evaporated in vacuo. The residue was triturated with hexane and dried in vacuo to give-sodium 4-{4-[(2S)-3-hydroxy-2-((2S)-2-hydroxy-3-phenoxypropylamino)-propyl]phenoxy}quinoline-7-carboxylate (14 mg).

NMR (DMSO-d₆, δ): 2.55-2.9 (5H, m), 3.1-3.5 (2H, m), 3.7-4.0 (3H, m), 6.5-6.55 (1H, m), 6.85-6.95 (3H, m), 7.1-7.5 (6H, m), 8.1-8.8 (4H, m)

### EXAMPLE 63

Under nitrogen, to a solution of 4-{(2S)-2-[benzyl-((2S)-2-hydroxy-3-phenoxypropyl)amino]-3-hydroxypropyl}-phenol (150 mg) in dimethyl sulfoxide (5 ml) was added potassium tert-butoxide (41 mg) at room temperature, and the mixture was stirred at the same temperature for 30 minutes. To this one was added 4-chloroquinoline-7-carboxylic acid amide (76 mg), and the mixture was stirred at 100° C. for 3.5 hours. The resulting mixture was poured into water and the aqueous mixture was extracted with ethyl acetate. The organic layer was washed with brine, dried over anhydrous magnesium sulfate, and evaporated in vacuo. The residue was purified by column chromatography on silica gel (chloroform:methanol=100:3 to 20:1) to give 4-(4-{(2S)-2-[benzyl-((2S)-2-hydroxy-3-phenoxypropyl)amino]-3-hydroxypropyl}phenoxy)quinoline-7-carboxylic acid amide (44 mg).

NMR (CDCl₃, δ): 2.6-3.35 (5H, m), 3.5-4.1 (7H, m), 6.60 (1H, d, J=5.2 Hz), 6.8-6.9 (2H, m), 6.96 (1H, d, J=7.3 Hz), 7.10 (2H, d, J=8.5 Hz), 7.2-7.4 (9H, m), 8.09 (1H, ABq, J=1.6, 8.9 Hz), 8.45-8.55 (2H, m), 8.71 (1H, d; J=5.2 Hz)

### EXAMPLE 64

Under nitrogen, to a solution of N-(2-benzyloxy-5-{(1R)-1-hydroxy-2-[(1S)-1-hydroxymethyl-2-(4-phenylsulfanylphenyl)-ethylamino]ethyl}phenyl)methanesulfonamide (90 mg) in dichloromethane (5 ml) was added dropwise boron tribromide (1M in dichloromethane, 1.2 ml) at 5° C., and the mixture was stirred at the same temperature for 20 minutes. The reaction mixture was poured into ice-cold water, and the aqueous mixture was extracted with ethyl acetate. The organic layer was dried over anhydrous magnesium sulfate and evaporated in vacuo. To the residue was added 4N hydrogen chloride in 1,4-dioxane in order to decompose the boran complexes, followed by evaporation in vacuo. The residue was dissolved into a mixture of saturated aqueous sodium hydrogencarbonate and dichloromethane. After separation, the organic layer was dried over anhydrous magnesium sulfate and evaporated in vacuo. The residue was purified by preparative thin layer chromatography (silica gel, chloroform:methanol=5:1) to give N-(2-hydroxy-5-{(1R)-1-hydroxy-2-[(1S)-1-hydroxymethyl-2-(4-phenylsulfanylphenyl)ethylamino]ethyl}phenyl)methanesulfonamide (4 mg).

NMR (CD₃OD, δ): 1.9-2.1 (2H, m), 2.65-3.1 (6H, m), 3.3-3.7 (2H, m), 4.55-4.75 (1H, m), 6.86 (1H, d, J=8.2 Hz), 7.05-7.4 (11H, m)

### EXAMPLE 65

To a solution of (S)-2-amino-3-[4-(quinolin-4-yloxy)-phenyl]propan-1-ol dihydrochloride (916 mg, 2.49 mmol) in ethanol (18 ml) was successively added (S)-3-phenoxy-1,2-epoxypropane (374 mg, 2.49 mmol) and diisopropylethylamine (2.17 ml, 12.5 mmol) at room temperature and the whole was refluxed for 11 hours. After cooling to room temperature, the solvent was evaporated and the residue was dissolved in ethyl acetate (20 ml). The solution was washed with water (20 ml×2), brine (20 ml×1), dried (magnesium sulfate), and evaporated to give a crude oil (995 mg). The crude oil was chromatographed on a 50 g of silica gel (eluent: chloroform/methanol=9/1) to give (2S)-2-[(2S)-2-hydroxy-3-phenoxypropylamino]-3-[4-(quinolin-4-yloxy)phenyl]propan-1-ol (306 mg, 28%) as a white solid.

IR (KBr): 3421 (br, OH, NH), 1500, 1250, 1213 cm⁻¹

NMR (CDCl₃, δ): 2.02 (3H, br), 2.80-3.06 (5H, m), 3.47 (1H, dd, J=5.1, 11.0 Hz), 3.71 (1H, dd, J=3.7, 11.0 Hz), 4.01 (3H, m), 6.54 (1H, d, J=5.2 Hz), 6.89-7.00 (3H, m), 7.13 (2H, d, J=8.4 Hz), 7.26-7.33 (4H, m), 7.55-7.81 (2H, m), 8.10 (1H, d, J=8.4 Hz), 8.36 (1H, d, J=7.5 Hz), 8.65 (1H, d, J=5.2 Hz)

MS: 445 (M+1)

### EXAMPLE 66

A solution of (S)-2-amino-3-[4-(imidazo[1,2-a]pyridin-5-yloxy)phenyl]propan-1-ol (313 mg, 1.10 mmol) and (S)-3-phenoxy-1,2-epoxypropane (198 mg, 1.32 mmol) in ethanol (6.0 ml) was refluxed for 4 hours. After cooling to room temperature, the solvent was evaporated and the residue was dissolved in ethyl acetate (20 ml). The solution was washed with water (20 ml×2), brine (20 ml×1), dried (magnesium sulfate), and evaporated to give an orange oil. The oil was chromatographed on a 50 g of silica gel (eluent: chloroform/methanol=95/5) to give a pale yellow oil (134 mg). Further purification was performed by a recycling preparative HPLC equipped with a GPC column (eluent: chloroform) to give 2(S)-2-[(2S)-2-hydroxy-3-phenoxypropyl-amino]-3-[4-(imidazo[1,2-a]pyridin-5-yloxy)phenyl]propan-1-ol (67.5 mg, 14%) as a pale yellow oil.

MS: 434 (M+1)

### EXAMPLE 67

To a solution of 2(S)-2-[(2S)-2-hydroxy-3-phenoxypropylamino]-3-[4-(imidazo[1,2-a]pyridin-5-yloxy)phenyl]propan-1-ol (63.6 mg, 0.147 mmol) in dioxane (1 ml) was added 4N hydrogen chloride in dioxane (1 ml) and the solution was stirred at room temperature for 1 hour. The solvent was removed by evaporation and the residue was dissolved in water (10 ml). The aqueous solution was lyophilized to give 2(S)-2-[(2S)-2-hydroxy-3-phenoxypropylamino]-3-[4-(imidazo[1,2-a]pyridin-5-yloxy)phenyl]propan-1-ol dihydrochloride (50.4 mg, 68%) as a pale yellow solid.

MS: 434 (M−CHl—Cl⁺)

### EXAMPLE 68

To a solution of N-[2-benzyloxy-5-[(1R)-2-[(1S)-2-hydroxy-1-(4-(imidazo[1,2-a]pyridin-5-yloxy)benzyl]ethylamino]-1-(triethylsilyloxy)ethyl]phenyl}methanesulfonamide (137 mg, 0.191 mmol) in tetrahydrofuran (1.4 ml) was added a solution of tetrabutylammonium fluoride in tetrahydrofuran (1.0M solution, 0.2 ml, 0.2 mmol) at room temperature and the mixture was stirred at the same temperature for 4 hours. The reaction mixture was diluted with ethyl acetate (10 ml) and washed with water (10 ml×1), brine (10 ml×1), dried (magnesium sulfate), and evaporated to give a yellow paste (119 mg). The crude oil was chromatographed on a 50 g of silica gel (eluent: chloroform/methanol=98/2 to 95/5) to give N-[2-benzyloxy-5-[(1R)-1-hydroxy-2-[(1S)-2-hydroxy-1-[4-(imidazo[1,2-a]pyridin-5-yloxy)benzyl]ethylamino]ethyl]-phenyl]methanesulfonamide (74.0 mg, 64%) as a pale yellow paste. The product was used immediately in the next step.

### EXAMPLE 69

A mixture of N-[2-benzyloxy-5-[(1R)-1-hydroxy-2-[(1S)-2-hydroxy-1-[4-(imidazo[1,2-a]pyridin-5-yloxy)benzyl]-ethylamino]ethyl]phenyl]methanesulfonamide (74.0 mg, 0.124 mmol), palladium (10% on activated carbon, 50% wet, 50 mg) and methanol (4.0 ml) was hydrogenated (1 atm) for 90 minutes. The catalyst was removed by filtration using Celite and washed with methanol. The filtrate was concentrated in vacuo to give N-[2-hydroxy-5-[(1R)-1-hydroxy-2-[(1S)-2-hydroxy-1-[4-(imidazo[1,2-a]pyridin-5-yloxy)benzyl]-ethylamino]ethyl]phenyl]methanesulfonamide (50.8 mg, 81%) as a white solid.

IR (KBr) 3423, 1502, 1153 cm⁻¹

MS: 513 (M+1)

### EXAMPLE 70

To a solution of (S)-2-[4-(2-amino-3-hydroxypropyl)-phenoxy]-N,N-dimethylnicotinamide hydrochloride (439 mg, 1.13 mmol) in ethanol (8.0 ml) were successively added diisopropylethylamine (492 μl, 2.82 mmol) and (S)-2-(3-chlorophenoxymethyl)oxirane (250 mg, 1.35 mmol) at room temperature. The solution was refluxed for 2 hours. After cooling to room temperature, the solvent was evaporated and the residue was dissolved in ethyl acetate (20 ml). The solution was washed with water (20 ml×2), brine (20 ml×1), dried (magnesium sulfate), and evaporated to give a yellow oil (460 mg). The crude oil was chromatographed on a 14 g of silica gel (eluent: chloroform/methanol=95/5 to 9/1) to give 2-[4-[2-[(2S)-3-(3-chlorophenoxy)-2-(hydroxy)-propylamino]-3-hydroxypropyl]phenoxy]-N,N-dimethylnicotinamide (160 mg, 26%) as a white foam.

IR (KBr): 3425, 1626, 1593, 1419 cm⁻¹

NMR (CDCl₃, δ): 2.06 (3H, br), 2.64-2.86 (5H, m), 3.04 (3H, s), 3.15 (3H, s), 3.46 (1H, dd, J=4.3, 10.7 Hz), 3.68 (1H, dd, J=3.5, 10.7 Hz), 3.72-3.88 (3H, m), 6.74 (1H, dd, J=2.3, 8.3 Hz), 6.85-7.26 (8H, m), 7.75 (1H, dd, J=1.8, 7.4 Hz, ArH), 8.12 (1H, dd, J=1.8, 5.0 Hz)

MS: 500 (M+1)

### EXAMPLE 71

To a solution of (S)-2-amino-3-[4-(quinolin-4-yloxy)-phenyl]propan-1-ol dihydrochloride (441 mg, 1.20 mmol) in 1,3-dimethyl-2-imidazolidinone (4.5 ml) were successively added (R)-N-[2-benzyloxy-5-[2-iodo-1-(triethylsilyloxy)-ethyl]phenyl]methanesulfonamide (809 mg, 1.44 mmol) and diisopropylethylamine (836 μl, 4.80 mmol) and the mixture was stirred at 100° C. for 28 hours. After cooling to room temperature, the mixture was diluted with ethyl acetate (20 ml) and washed with water (20 ml×2), brine (20 ml×1), dried (magnesium sulfate), and evaporated to give a yellow oil (736 mg). The oil was dissolved in tetrahydrofuran (7.0 ml), and a solution of tetrabutylammonium fluoride in tetrahydrofuran (1.0M, 1.44 ml) was added dropwise to this solution at room temperature. After stirring for 1 hour, the mixture was diluted with ethyl acetate (20 ml), washed with water (20 ml×2), brine (20 ml×1), dried (magnesium sulfate) and evaporated to give a yellow oil (636 mg). The crude oil was chromatographed on a 50 g of silica gel (eluent: chloroform/methanol=97/3 to 95/5 then 9/1) to give N-[2-benzyloxy-5-[(1R)-1-hydroxy-2-[(1S)-2-hydroxy-1-[4-(quinolin-4-yloxy)benzyl]ethylamino]ethyl]phenyl]methanesulfonamide (24.3 mg, 3.3%) as a pale yellow paste. The product was immediately used in the next step.

### EXAMPLE 72

A mixture of N-[2-benzyloxy-5-[(1R)-1-hydroxy-2-[(1S)-2-hydroxy-1-[4-(quinolin-4-yloxy)benzyl]ethylamino]ethyl]-phenyl]methanesulfonamide (24.3 mg, 0.0396 mmol), palladium (10% on activated carbon, 50% wet, 20 mg) and methanol (2.0 ml) was hydrogenated (1 atm) at room temperature for 2 hours. The catalyst was removed by filtration using Celite and washed with methanol. The filtrate was concentrated in vacuo to give N-[2-hydroxy-5-[(1R)-1-hydroxy-2-[(1S)-2-hydroxy-1-[4-(quinolin-4-yloxy)benzyl]ethylamino]ethyl]phenyl}methanesulfonamide (20.0 mg, 97%) as a colorless paste.

IR (KBr): 3423, 1618, 1591, 1504, 1306, 1151 cm⁻¹

MS: 524 (M+1)

### EXAMPLE 73

To a suspension of (S)-2-amino-3-[4-(7-chloroquinolin-4-yloxy)phenyl]propan-1-ol hydrochloride (480 mg, 1.19 mmol) in ethanol (10 ml) were successively added diisopropylamine (0.518 ml, 2.97 mmol) and (S)-3-phenoxy-1,2-epoxypropane (197 mg, 1.31 mmol) at room temperature and the solution was refluxed for 2.5 hours. After cooling to room temperature, the solvent was removed by evaporation and the residue was suspended in ethyl acetate (50 ml). The mixture was washed with water (50 ml×1), brine (50 ml×1), dried (magnesium sulfate), and evaporated to give a yellow oil (480 mg). The crude oil was chromatographed on a 50 g of silica gel (eluent: chloroform/methanol=95/5) to give 2(S)-3-[4-(7-chloroquinolin-4-yloxy)phenyl]-2-[(2S)-2-hydroxy-3-(phenoxy)-propylamino]propan-1-ol (176 mg, 31%) as a white solid.

IR (KBr): 3381, 1612, 1587, 1570, 1495, 1246, 1211 cm⁻¹

NMR (CDCl₃, δ): 2.02 (3H, br s), 2.78-3.02 (5H, m), 3.47 (1H, dd, J=5.0, 10.8 Hz), 3.71 (1H, dd, J=3.3, 10.8 Hz), 4.01-4.05 (3H, m), 6.52 (1H, d, J=5.2 Hz), 6.89-7.00 (3H, m), 7.25-7.33 (4H, m), 7.53 (1H, dd, J=2.0, 8.9 Hz), 8.09 (1H, d, J=2.0 Hz), 8.30 (1H, d, J=8.9 Hz), 8.64 (1H, d, J=5.2 Hz)

MS (m/z): 479 (M+1)

### EXAMPLE 74

To a suspension of (S)-2-amino-3-[4-(7-chloroquinolin-4-yloxy)phenyl]propan-1-ol hydrochloride (400 mg, 1.10 mmol) in ethanol (10 ml) was added successively diisopropylethylamine (0.433 ml, 2.49 mmol) and (R)-2-(3-chlorophenyl)oxirane (154 mg, 0.996 mmol) and the mixture was refluxed for 9 hours. After cooling to room temperature, the solvent was removed by evaporation and the residue was suspended in ethyl acetate (20 ml). The suspension was washed with water (20 ml×2), brine (20 ml×1), dried (magnesium sulfate), and evaporated to give a yellow oil (658 mg). The crude oil was purified by a recycling preparative HPLC equipped with a GPC column (eluent: chloroform/methanol=99.5/0.5) to give (2S)-2-((2R)-2-(3-chlorophenyl)-2-(hydroxy)ethylamino]-3-[4-(7-chloroquinolin-4-yloxy)phenyl]propan-1-ol (50.6 mg, 10%) as a white foam.

IR (KBr): 3421, 2929, 1614, 1570, 1421, 1209 cm⁻¹

NMR (CDCl₃, δ): 1.78 (3H, br), 2.45 (1H, dd, J=10.7, 12.9 Hz), 2.83 (1H, dd, J=3.0, 12.9 Hz), 2.88-2.95 (2H, m), 3.23 (1H, m), 3.71-3.92 (2H, m), 4.17-4.27 (1H, m), 6.56 (1H, d, J=5.2 Hz), 7.14-7.38 (8H, m), 7.51 (1H, dd, J=2.1 Hz), 8.06 (1H, d, J=2.1 Hz), 8.30 (1H, d, J=8.9 Hz), 8.47 (1H, d, J=5.2 Hz)

MS (m/z): 483, 485 (M+1)

### EXAMPLE 75

Potassium hydroxide powder (85% purity, 35.3 mg, 0.535 mmol) was added to dimethyl sulfoxide (5.0 ml) at room temperature and the mixture was stirred at the same temperature for 1 hour. To the mixture was added 4-[(2S)-2-[benzyl[(2R)-2-(3-chlorophenyl)-2-hydroxyethyl]amino]-3-hydroxypropyl]phenol (200 mg, 0.486 mmol) and stirred for 30 minutes. Further, a solution of 4-chloroquinoline (103 mg, 0.203 mmol) in dimethyl sulfoxide (0.5 ml) was added and the mixture was stirred at 100° C. for 5 hours. After cooling to room temperature, the mixture was diluted with ethyl acetate (20 ml) and washed with water (20 ml×2), brine (20 ml×1), dried (magnesium sulfate), then evaporated to give a yellow solid (292 mg). The crude solid was purified by a recycling preparative HPLC equipped with a GPC column (eluent: chloroform/triethylamine=99.5/0.5) to give (2S)-2-[benzyl-[(2R)-2-(3-chlorophenyl)-2-hydroxyethyl]amino]-3-[4-(quinolin-4-yloxy)phenyl]propan-1-ol (155 mg, 59%) as a white solid.

MS (m/z): 539 (M+1)

### EXAMPLE 76

To a solution of (2S)-2-[benzyl[(2R)-2-(3-chlorophenyl)-2-hydroxyethyl]amino]-3-(4-(quinolin-4-yloxy)phenyl]propan-1-ol (148 mg, 0.275 mmol) in a mixed solvent of methanol (3.0 ml) and chlorobenzene (3.0 ml) was added palladium (10% on activated carbon, 50% wet, 70 mg) and the mixture was hydrogenated (1 atm) for 90 minutes. The catalyst was filtered off using Celite and washed with methanol. The filtrate was concentrated in vacuo to give 2-[(2R)-2-(3-chlorophenyl)-2-hydroxyethylamino]-3-[4-(quinolin-4-yloxy)-phenyl]propan-1-ol (134 mg, 109%) as a pale yellow solid.

MS (m/z): 449 (M+1)

### EXAMPLE 77

Potassium hydroxide powder (85% purity, 53.4 mg, 0.809 mmol) was added to dimethyl sulfoxide (6.0 ml) at room temperature and the mixture was stirred at the same temperature for 80 minutes. To the mixture was added 4-[(2S)-2-[benzyl[(2S)-2-hydroxy-3-phenoxypropyl]amino]-3-hydroxypropyl]phenol (300 mg, 0.736 mmol) and stirred for 30 minutes. Further, 4-chloro-7-methoxyquinoline (171 mg, 0.883 mmol) was added and the mixture was stirred at 100° C. for 3.5 hours. After cooling to room temperature, the mixture was quenched by the addition of water (30 ml) and extracted with ethyl acetate (30 ml×1). The organic layer was separated and washed with water (30 ml×2), brine (30 ml×1), dried (magnesium sulfate), then evaporated to give a brown paste (437 mg). The crude paste was chromatographed on a 50 g of silica gel (eluent: hexane/ethyl acetate=1/1 to 1/2) to give (2S)-2-[benzyl[(2S)-2-hydroxy-3-phenoxypropyl]amino]-3-[4-(7-methoxyquinolin-4-yloxy)phenyl]propan-1-ol (195 mg, 47%) as a white foam.

NMR (CDCl₃, δ): 1.82 (2H, br), 2.62 (1H, dd; J=8.5, 13.6 Hz), 2.79-3.21 (4H, m), 3.53-4.00 (8H, m), 6.41 (1H, d, J=5.3 Hz), 6.82-7.42 (16H, m), 8.24 (1H, d, J=9.2 Hz), 8.57 (1H, d, J=5.3 Hz)

MS (m/z): 565 (M+1)

### EXAMPLE 78

To a solution of (2S)-2-[benzyl[(2S)-2-hydroxy-3-phenoxypropyl]amino]-3-[4-(7-methoxyquinolin-4-yloxy)phenyl]-propan-1-ol (184 mg, 0.326 mmol) in methanol (4.0 ml)) was added palladium (10% on activated carbon, 50% wet, 184 mg) and the mixture was hydrogenated (1 atm) for 2.5 hours. The catalyst was removed by filtration using Celite and washed with methanol. The filtrate was concentrated to give 2(S)-2-[(2S)-2-hydroxy-3-(phenoxy)propylamino)-3-[4-(7-methoxyquinolin-4-yloxy)phenyl]propan-1-ol (135 mg, 87%) as a white solid.

IR (KBr): 3421, 1623, 1-583, 1500, 1429, 1311, 1228 cm⁻¹

NMR (CDCl₃, δ): 2.78 (3H, br), 2.89-3.18 (5H, m), 3.57 (1H, dd, J=5.6, 11.4 Hz), 3.78 (1H, dd, J=3.4, 11.4 Hz), 3.97 (3H, s), 4.01 (2H, d, J=5.2 Hz), 4.56 (1H, m), 6.41 (1H, d, J=5.3 Hz), 6.91-6.99 (3H, m), 7.10 (2H, d, J=8.4 Hz), 7.23-7.32 (5H, m), 7.41 (1H, d, J=2.4 Hz), 8.21 (1H, d, J=9.2 Hz), 8.56 (1H, d, J=5.3 Hz)

MS (m/z): 475 (M+1)

### EXAMPLE 79

Potassium hydroxide powder (85% purity, 53.4 mg, 0.809 mmol) was added to dimethyl sulfoxide (6.0 ml) at room temperature and the mixture was stirred at the same temperature for 1 hour. To the mixture was added 4-[(2S)-2-(benzyl[(2S)-2-hydroxy-3-phenoxypropyl]amino]-3-hydroxypropyl]phenol (300 mg, 0.736 mmol) and stirred for 40 minutes. Further, 4-chloro-6-fluoroquinoline (160 mg, 0.881 mmol) was added and the mixture was stirred at 100° C. for 24 hours. After cooling to room temperature, the mixture was quenched by the addition of water (20 ml) and extracted with ethyl acetate (20 ml×1). The organic layer was separated and washed with water (20 ml×2), brine (20 ml×1), dried (magnesium sulfate), then evaporated to give a pale brown paste (424 mg). The crude paste was chromatographed on a 20 g of silica gel (eluent: hexane/ethyl acetate=2/1 to 1/1) to give (2S)-2-[benzyl[(2S)-2-hydroxy-3-phenoxypropyl]amino]-3-[4-(6-fluoroquinolin-4-yloxy)phenyl]propan-1-ol (195 mg, 48%) as a white foam.

NMR (CDCl₃, δ): 1.62 (2H, br), 2.58-3.22 (5H, m), 3.54-4.00 (5H, m), 6.54 (1H, d, J=5.2 Hz), 6.82-7.30 (14H, m), 7.47-7.57 (1H, m), 7.96 (1H, dd, J=2.9, 9.4 Hz), 8.09 (1H, dd, J=5.3, 9.4 Hz), 8.62 (1H, d, J=5.2 Hz)

MS (m/z): 553 (M+1)

### EXAMPLE 80

To a solution of (2S)-2-[benzyl[(2S)-2-hydroxy-3-phenoxypropyl]amino]-3-[4-(6-fluoroquinolin-4-yloxy)phenyl]-propan-1-ol (182 mg, 0.329 mmol) in methanol (4.0 ml) was added palladium (10% on activated carbon, 50% wet, 182 mg) and the mixture was hydrogenated (1 atm) for 6 hours. The catalyst was removed by filtration using Celite and washed with methanol. The filtrate was concentrated to give 2(S)-3-[4-(6-fluoroquinolin-4-yloxy)phenyl]-2-[(2S)-2-hydroxy-3-(phenoxy)propylamino]propan-1-ol (145 mg, 95%) as a white solid.

IR (KBr): 3381, 1599, 1502, 14.66, 1296, 1215 cm⁻¹

NMR (CDCl₃, δ): 2.91-3.26 (8H, m), 3.63 (1H, dd, J=5.6, 11.6 Hz), 3.73 (1H, dd, J=3.0, 11.6 Hz), 4.03 (1H, d, J=4.9 Hz), 4.35 (1H, m), 6.53 (1H, d, J=5.2 Hz), 6.86-6.99 (3H, m), 7.09 (2H, d, J=8.3 Hz), 7.22-7.31 (2H, m), 7.33 (2H, d, J=8.3 Hz), 7.51 (1H, dt, J=2.8, 8.7 Hz), 7.92 (1H, dd, J=2.9, 9.3 Hz), 8.09 (1H, dd, J=5.3, 9.3 Hz), 8.59 (1H, d, J=5.2 Hz)

MS (m/z): 463 (M+1)

### EXAMPLE 81

Potassium hydroxide powder (85% purity, 55.0 mg, 0.833 mmol) was added to dimethyl sulfoxide (8.0 ml) at room temperature and the mixture was stirred at the same temperature for 1 hour. To the mixture was added 4-[(2S)-2-[benzyl[(2R)-2-(4-benzyloxy-3-nitrophenyl)-2-hydroxyethyl]-amino]-3-hydroxypropyl]phenol (400 mg, 0.757 mmol) and stirred for 40 minutes. Further, 4-chloro-6-fluoroquinoline (179 mg, 0.986 mmol) was added and the mixture was stirred at 100° C. for 96 hours. After cooling to room temperature, the mixture was diluted with ethyl acetate (20 ml) and washed with water (20 ml×3), brine (20 ml×1), dried. (magnesium sulfate), then evaporated to give a brown foam (468 mg). The crude product was chromatographed on a 25 g of silica gel (eluent: hexane/ethyl acetate=1/1, then chloroform/methanol=9/1) to give (2S)-2-[benzyl[(2R)-2-(4-benzyloxy-3-nitrophenyl)-2-hydroxyethyl]amino]-3-[4-(6-fluoroquinolin-4-yloxy)phenyl]propan-1-ol (90.7 mg, 18%) as an orange foam. The product was immediately used in the next step.

### EXAMPLE 82

To a solution of (2S)-2-[benzyl[(2R)-2-(4-benzyloxy-3-nitrophenyl)-2-hydroxyethyl]amino]-3-[4-(6-fluoroquinolin-4-yloxy)phenyl]propan-1-ol (90.7 mg, 0.135 mmol) in a mixed solvent of ethanol (6.0 ml) and water (2.0 ml) were successively added iron powder (22.6 mg, 0.405 mmol) and ammonium chloride (3.6 mg, 0.067 mmol). The mixture was refluxed for 1 hour, with vigorous stirring. After cooling to room temperature, the metal was removed by filtration using Celite, and washed with ethanol. The filtrate was concentrated in vacuo to give a pale brown solid. To the solid were added aqueous saturated sodium hydrogencarbonate solution (20 ml) and ethyl acetate (20 ml), and the whole was stirred vigorously. The organic layer was separated and washed with water (20 ml×2), brine (20 ml×1), dried (magnesium sulfate), and evaporated to give (2S)-2-[[(2R)-2-(3-amino-4-benzyloxyphenyl)-2-hydroxyethyl]benzylamino]-3-[4-(6-fluoroquinolin-4-yloxy)phenyl]propan-1-ol (89.1 mg, 103%) as a yellow foam. The product was immediately used in the next step.

### EXAMPLE 83

To a solution of (2S)-2-[[(2R)-2-(3-amino-4-benzyloxyphenyl)-2-hydroxyethyl]benzylamino]-3-[4-(6-fluoroquinolin-4-yloxy)phenyl]propan-1-ol (89.1 mg, 0.138 mmol) in dichloromethane (2.0 ml) was added pyridine (33.4 μl, 0.412 mmol) and the solution was cooled to 0° C. To the solution was added methanesulfonyl chloride (25.6 μl, 0.330 mmol) at 0° C. and stirred at the same temperature for 1 hour. The reaction mixture was warmed to room temperature and stirred for 15 minutes. The reaction mixture was diluted with ethyl acetate (10 ml) and washed with water (10 ml×2), brine (10 ml×1), dried over magnesium sulfate. Evaporation of the solvent gave an orange foam (97.6 mg). The crude product was chromatographed on a 25 g of silica gel (eluent: chloroform/methanol=98/2) to give N-[5-[(1R)-2-[benzyl-[(1S)-1-[4-(6-fluoroquinolin-4-yloxy)benzyl]-2-hydroxyethyl]-amino]-1-hydroxyethyl]-2-benzyloxyphenyl]methanesulfonamide (30.0 mg, 30%) as a pale yellow foam. The product was immediately used in the next step.

### EXAMPLE 84

To a solution of N-[5-[(1R)-2-[benzyl[(1S)-1-[4-(6-fluoroquinolin-4-yloxy)benzyl]-2-hydroxyethyl]amino]-1-hydroxyethyl]-2-benzyloxyphenyl]methanesulfonamide. (30.0 mg, 0.0416 mmol) in methanol (1.0 ml) was added palladium (10% on activated carbon, 50% wet, 30 mg) and the mixture was hydrogenated (1 atm) for 1 hour. The catalyst was removed by filtration using Celite and washed with methanol. The filtrate was concentrated to give N-[2-hydroxy-5-[(1R)-1-hydroxy-2-[(1S)-1-[4-(6-fluoroquinolin-4-yloxy)benzyl]-2-hydroxyethylamino]ethyl]phenyl]methanesulfonamide (15.2 mg, 68%) as a pale yellow solid.

IR (KBr): 3419, 1599, 1510, 1468, 1294, 1151 cm⁻¹

MS (m/z): 542 (M+1)

### EXAMPLE 85

The following compound was obtained by a similar manner to that of Example 94 followed by a reduction of the nitro group as described in Example 88.

(S)-1-(3-Amino-4-benzyloxyphenoxy)-3-((S)-N-benzyl-[1-hydroxy-3-[4-(2-pyridinyloxy)phenyl]-2-propyl]amino]-2-propanol

MS (m/z): 606 (M+1)

### EXAMPLE 86

The following compound was obtained by a similar manner as described in Example 89.

(S)-1-(4-Hydroxy-3-methanesulfonylaminophenoxy)-3-[(S)-[1-hydroxy-3-[4-(pyridin-2-yloxy)phenyl]-2-propyl]amino]-2-propanol

IR (KBr): 1649 (m), 1512 (s), 1468 (m), 1429 (m) cm⁻¹

NMR (CD₃OD, δ): 2.6-2.9 (5H, m), 2.90 (3H, s), 3.3-3.6 (2H, m), 3.8-3.9 (2H, m), 3.9-4.0 (1H, m), 6.61 (1H, d, J=11.5 Hz), 6.76 (1H, d, J=11.7 Hz), 6.88 (1H, d, J=8.1 Hz), 6.9-7.2 (3H, m), 7.2-7.3 (3H, m), 7.79 (1H, t, J=7.4 Hz), 8.12 (1H, d, J=6 Hz)

MS (m/z): 504 (M+1)

### EXAMPLE 87

A mixture of (S)-2-[(4-benzyloxy-3-nitrophenoxy)methyl]-oxirane (123 mg), (S)-2-amino-3-[4-(3-methyl-2-pyridinyloxy)-phenyl]propanol (96 mg) and methanol (2 ml) was heated under reflux for 3 hours and evaporated. The residue was purified by a column chromatography (silica gel, dichloromethane:methanol:concentrated ammonia solution=20:1:0.1) to afford (S)-1-(4-benzyloxy-3-nitrophenoxy)-3-[(S)-1-hydroxy-3-[4-(3-methyl-2-pyridinyloxy)phenyl]-2-propylamino]-2-propanol (91.2 mg).

MS (m/z): 560 (M+1)

### EXAMPLE 88

To a mixture of (S)-1-(4-benzyloxy-3-nitrophenoxy)-3-[(S)-1-hydroxy-3-[4-(3-methyl-2-pyridinyloxy)phenyl]-2-propylamino]-2-propanol (86 mg), tetrahydrofuran (5 ml) and saturated aqueous sodium bicarbonate solution (5 ml), benzyloxycarbonyl chloride (26 μl) was added and the resulting mixture was stirred at room temperature for 1 hour. The reaction mixture was extracted with ethyl acetate (5 ml×2). The extract was washed with water (5 ml×2) and evaporated to afford (S)-1-(4-benzyloxy-3-nitrophenoxy)-3-[N-benzyloxycarbonyl-[(S)-1-hydroxy-3-[4-(3-methyl-2-pyridinyloxy)phenyl]-2-propylamino]-2-propanol as a crude residue. The crude residue was dissolved in ethanol (3 ml) and heated with water (0.3 ml), iron powder (about 50 mg) and ammonium chloride (about 10 mg) under reflux for 1 hour. The reaction mixture was filtered and worked up by a similar manner to that described above to afford (S)-1-(3-amino-4-benzyloxyphenoxy)-3-[N-benzyloxycarbonyl-[(S)-1-hydroxy-3-[4-(3-methyl-2-pyridinyloxy)phenyl]-2-propylamino]-2-propanol (136 mg) as a crude product, which was used without any further purification.

MS (m/z): 664 (M+1).

### EXAMPLE 89

To a mixture of (S)-1-(3-amino-4-benzyloxyphenoxy)-3-[N-benzyloxycarbonyl-[(S)-1-hydroxy-3-[4-(3-methyl-2-pyridinyloxy)phenyl]-2-propylamino]-2-propanol (43.6 mg), pyridine (0.03 ml) and dichloromethane (1 ml), methanesulfonyl chloride (7 μl) was added at 0° C. After 40 minutes, additional methanesulfonyl chloride (7 μl) was added. After 1 hour, saturated aqueous sodium bicarbonate solution (5 ml) and ethyl acetate (5 ml) were added therein and the resulting mixture was stirred at room temperature for 1 hour. The organic layer was separated, washed successively with water (5 ml×2) and brine (5 ml×1), dried over magnesium sulfate and evaporated to afford (S)-1-(4-benzyloxy-3-methanesulfonylaminophenoxy)-3-[N-benzyloxycarbonyl-[(S)-1-hydroxy-3-[4-(3-methyl-2-pyridinyloxy)phenyl]-2-propylamino]-2-propanol, which was converted to (S)-1-(4-hydroxy-3-methanesulfonylaminophenoxy)-3-[(S)-1-hydroxy-3-[4-(3-methyl-2-pyridinyloxy)phenyl]-2-propylamino]-2-propanol (19.2 mg) by catalytic hydrogenation on palladium charcoal in a usual manner followed by preparative thin-layer chromatography (dichloromethane:methanol:concentrated ammonia solution=7:1:0.1).

IR (KBr): 3420 (broad s), 1510 (m), 1415 (m), 1213 (m) cm⁻¹

NMR (CD₃OD, δ): 2.32 (3H, s), 2.6-3.0 (5H, m), 2.91 (3H, s), 3.3-3.7 (2H, m), 3.8-4.0 (3H, m), 6.64 (1H, d, J=8.6 Hz), 6.78 (1H, d, J=8.6 Hz), 6.9-7.1 (4H, m), 7.25 (2H, d, J=8.4 Hz), 7.67 (1H, d, J=6 Hz), 7.89 (1H, d, J=4 Hz)

MS (m/z): 518 (M+1)

### EXAMPLE 90

To a solution of (S)-2-amino-3-[4-(3-hydroxymethyl-2-pyridinyloxy)phenyl]propanol dihydrochloride (112 mg) in methanol, 28% sodium methoxide-methanol solution (126 mg) was added and evaporated to afford the corresponding free base. A mixture of the free base, (R)-3-pyridyloxirane (117 mg) and isopropanol (4 ml) was heated under reflux for 6 hours, evaporated and purified by preparative thin-layer chromatography (dichloromethane:methanol:concentrated ammonia solution=5:1:0.1) to afford (R)-1-(3-pyridyl)-2-[(S)-1-hydroxy-3-[4-(3-hydroxymethyl-2-pyridinyloxy)phenyl]-2-propylamino]ethanol (16.1 mg).

IR (KBr): 1585 (m), 1425 (m), 1240 (s), 1045 (s) cm⁻¹

NMR (CD₃OD, δ): 2.7-3.2 (5H, m), 3.4-3.7 (2H, m), 40.73 (2H, s), 4.7-4.8 (1H, m), 7.00 (2H, d, J=8.1 Hz), 7.11 (1H, t, J=5.6 Hz), 7.25 (2H, d, J=8.1 Hz), 7.43 (1H, dd, J=5.6, 10.4 Hz), 7.85 (1H, d, J=7.8 Hz), 7.92 (2H, d, J=6.4 Hz), 8.44 (1H, d, J=3.7 Hz), 8.55 (1H, s)

MS (m/z): 396 (M+1)

### EXAMPLE 91

The following compound was obtained by a similar manner to that of Example 87.

(S)-1-(3-Pyridyloxy)-3-[(S)-1-hydroxy-3-[4-(3-hydroxymethyl-2-pyridinyloxy)phenyl]-2-propylamino]-2-propanol

IR (KBr): 1579 (m), 1427 (s), 1240 (s) cm⁻¹

NMR (CD₃OD, δ): 2.6-3.1 (5H, m), 3.4-3.7 (2H, m), 3.9-4.3 (3H, m), 4.73 (2H, s), 7.00 (2H, d, J=8.4 Hz), 7.11 (1H, t, J=6.6 Hz), 7.27 (2H, d, J=8.5 Hz), 7.3-7.5 (3H, m), 7.93 (1H, d, J=5.9 Hz), 8.12 (1H, d, J=3.3 Hz), 8.24 (1H, s)

MS (m/z): 426 (M+1)

### EXAMPLE 92

A mixture of (S)-1-phenoxy-3-[(S)-1-hydroxy-3-[4-(3-hydroxymethyl-2-pyridinyloxy)phenyl]-2-propylamino]-2-propanol dihydrochloride (29 mg), palladium hydroxide on charcoal (5 mg) and methanol (2 ml) was stirred in the presence of hydrogen (1 atm) at room temperature for 2 hours. The reaction mixture was filtered and evaporated to afford (S)-1-phenoxy-3-[(S)-1-hydroxy-3-[4-(3-methyl-2-pyridinyloxy)phenyl]-2-propylamino]-2-propanol dihydrochloride (1×9.7 mg)

IR (KBr): 1598 (m), 1502 (m), 1244 (s), 1049 (m) cm⁻¹

NMR (CD₃OD, δ): 2.35 (3H, s), 3.0-3.2 (2H, m), 3.4-3.9 (5H, m), 4.0-4.1 (2H, m), 4.2-4.4 (1H, m), 6.9-7.4 (10H, m), 7.84 (1H, d, J=7.1 Hz), 7.95 (1H, d, J=5.3 Hz)

MS (m/z): 409 (M+1, free)

### EXAMPLE 93

The following compound was obtained by a similar manner to that of Example 90.

(R)-1-(3-Pyridyl)-2-[(S)-1-hydroxy-3-[4-(3-methyl-2-pyridinyloxy)phenyl]-2-propylamino]ethanol

IR (KBr): 2924 (m), 1579 (s), 1415 (m) cm⁻¹

NMR (CDCl₃, δ): 2.31 (3H, s), 2.7-3.0 (5H, m), 3.4-3.7 (2H, m), 4.8-4.9 (1H, m), 6.9-7.0 (3H, m), 7.23 (2H, d, J=8.5 Hz), 7.4-7.5 (1H, m), 7.68 (1H, d, J=7.8 Hz), 7.8-7.9 (2H, m), 8.44 (1H, s), 8.54 (1H, s)

MS (m/z): 380 (M+1)

### EXAMPLE 94

The following compound was obtained in a similar manner to that of Example 87.

(S)-1-Phenoxy-3-[(S)-1-hydroxy-3-[4-(2-pyridinyloxy)-phenyl]-2-propylamino]-2-propanol

IR (KBr): 1593 (m), 1429 (m), 1244 (s), 1043 (s) cm⁻¹

NMR (CD₃OD, δ): 2.7-2.9 (3H, m), 2.9-3.1 (2H, m), 3.4-3.7 (2H, m), 3.9-4.0 (2H, m), 4.0-4.2 (1H, m), 6.9-7.1 (7H, m), 7.2-7.4 (4H, m), 7.79 (1H, t, J=7.1 Hz), 8.10 (1H, d, J=7 Hz)

MS (m/z): 395 (M+1)

### EXAMPLE 95

A mixture of 2-{4-[(2S)-3-hydroxy-2-((2S)-2-hydroxy-3-phenoxypropylamino)propyl]phenoxy}quinoline-3-carboxylic acid methyl ester (270 mg) and aqueous 28% ammonium hydroxide (5.0 ml) in 1,4-dioxane (5.0 ml) was stirred at room temperature for 2 days. The mixture was evaporated in vacuo, followed by partition between ethyl acetate and water. The organic layer was washed with brine, dried over sodium sulfate and evaporated in vacuo. To a solution of the residue in dioxane (3 ml) was added-4N hydrogen chloride in dioxane (3 ml) at room temperature, and the solution was stirred at the same temperature for 3 hours. The mixture was evaporated in vacuo, and the residue was triturated with diisopropyl ether to give 2-{4-[(2S)-3-hydroxy-2-((2S)-2-hydroxy-3-phenoxypropylamino)propyl]phenoxy}quinoline-3-carboxylic acid amide (0.41 g) as a colorless powder.

NMR (DMSO-d₆, δ): 2.80-3.73 (7H, m), 3.99-4.05 (2H, m), 4.20-4.30 (1H, m), 6.95-7.10 (3H, m), 7.05-8.10 (10H, m), 8.79 (1H, s)

MS (m/z): 488 (M+1)

### EXAMPLE 96

The following compound was synthesized according to a similar manner to that of Example 97.

N-(2-{4-[(2S)-3-Hydroxy-2-((2S)-2-hydroxy-3-phenoxypropylamino)propyl]phenoxy}pyridin-3-yl)methanesulfonamide as a Brown Powder

NMR (DMSO-d₆, δ): 2.90-3.60 (10H, m), 4.00-4.10 (2H, m), 4.20-4.30 (1H, m), 6.90-7.17 (6H, m), 7.25-7.40 (4H, m), 7.75-7.95 (2H, m), 9.60 (1H, br s)

MS (m/z): 488 (M+1)

### EXAMPLE 97

{(1S)-1-Hydroxymethyl-2-[4-(3-aminopyridin-2-yloxy)-phenyl]ethyl}-(2S)-(2-hydroxy-3-phenoxypropyl)carbamic acid tert-butyl ester (110 mg) and pyridine (0.1 ml) in dichloromethane (6 ml) under ice water cooling over 10 minutes and the mixture was stirred at room temperature for a further 1 hour. To this one was added aqueous saturated solution of sodium bicarbonate (5.0 ml). The mixture was stirred at the same temperature for 18 hours, and which was dissolved in ethyl acetate, washed with aqueous saturated sodium bicarbonate solution and brine, dried over sodium sulfate, and evaporated in vacuo. To a solution of the residue in dioxane (3 ml) was added 4N hydrogen chloride in dioxane (3 ml) at room temperature, and the solution was stirred at the same temperature for 3 hours. The mixture was evaporated in vacuo, and the residue was triturated with diisopropyl ether to give N-(2-{4-[(2S)-3-hydroxy-2-((2S)-2-hydroxy-3-phenoxypropylamino)propyl]phenoxy}pyridin-3-yl)benzenesulfonamide as a brown powder.

NMR (DMSO-d₆, δ): 2.90-3.60 (7H, m), 4.50-4.10 (2H, m), 4.20-4.30 (1H, m), 6.90-7.17 (11H, m), 7.24-7.40 (4H, m), 7.75-7.95 (2H, m)

MALDI-MS (m/z): 549 (M+Na)

1. A compound of the formula [I]: 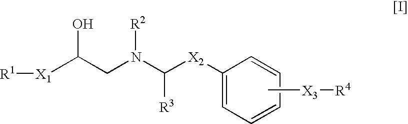
wherein
X₁ is bond or —OCH₂—;
X₂ is —(CH₂)ₙ—, in which n is 1, 2 or 3;
X₃ is bond, —O—, —S—, —OCH₂— or —NH—;
R¹ is phenyl or pyridyl, each of which may be substituted with one or two substituent(s) selected from the group consisting of hydroxy, halogen, amino, [(lower)alkylsulfonyl]amino, nitro, benzyloxycarbonylamino and benzyloxy;
R² is hydrogen, (lower)alkoxycarbonyl, benzyl or benzyloxycarbonyl;
R³ is hydroxy(lower)alkyl, (lower)alkoxy(lower)alkyl or halo(lower)alkyl; and
R⁴ is aryl or an unsaturated heterocyclic group containing nitrogen, each of which may be substituted with one or two substituent(s) selected from the group consisting of hydroxy, lower alkyl, lower alkoxy, halo(lower)alkyl, halogen, hydroxy(lower)alkyl, (lower)alkoxy(lower)alkyl, cyano, carboxy, (lower)alkoxycarbonyl, lower alkanoyl, carbamoyl, (mono or di)(lower)-alkylcarbamoyl, [(lower)alkylsulfonyl]carbamoyl, amino, nitro, ureido, [(lower)alkylcarbonyl]amino, [(lower)alkylsulfonyl]amino and (arylsulfonyl)amino,
and a salt thereof.

2. A compound of claim 1, wherein
X₁ is bond or —OCH₂—;
X₂ is —(CH₂)ₙ—, in which n is 1, 2 or 3;
X₃ is bond, —O—, —S—, —OCH₂— or —NH;
R¹ is phenyl or pyridyl, each of which may be substituted with one or two substituent(s) selected from the group consisting of hydroxy, halogen, amino, [(lower)alkylsulfonyl]amino, nitro, benzyloxycarbonylamino and benzyloxy;
R² is hydrogen, (lower)alkoxycarbonyl, benzyl or benzyloxycarbonyl;
R³ is hydroxy(lower)alkyl, (lower)alkoxy(lower)alkyl or halo(lower)alkyl; and
R⁴ is phenyl, naphthyl or an unsaturated 5 or 6 membered heteromonocyclic group containing 1 to 4 nitrogen atom(s) or an unsaturated condensed heterocyclic group containing 1 to 4 nitrogen atom(s), each of which may be substituted with one or two substituent(s) selected from the group consisting of hydroxy, lower alkyl, lower alkoxy, halo(lower)alkyl, halogen, hydroxy(lower)alkyl, (lower)alkoxy(lower)alkyl, cyano, carboxy, (lower)alkoxycarbonyl, lower alkanoyl, carbamoyl, (mono or di)(lower)alkylcarbamoyl, [(lower)alkylsulfonyl]carbamoyl, amino, nitro, ureido, [(lower)alkylcarbonyl]amino, [(lower)alkylsulfonyl]amino and (arylsulfonyl)amino.

3. A compound of claim 2, wherein
X₁ is bond or —OCH₂—;
X₂ is —(CH₂)ₙ— in which n is 1;
X₃ is bond, —O— or —S—;
R¹ is phenyl which may be substituted with one or two substituent(s) selected from the group consisting of halogen, nitro, amino, benzyloxy, benzyloxycarbonylamino, hydroxy and [(lower)alkylsulfonyl]amino; or pyridyl which may be substituted with amino
R² is hydrogen or (lower)alkoxycarbonyl;
R³ is hydroxy(lower)alkyl; and
R⁴ is phenyl, naphthyl, pyridyl, pyridyl N-oxide, pyrrolyl, pyrazinyl, quinolyl, isoquinolyl, imidazopyridyl, benzothiazolyl, quinoxalinyl, acridinyl, pyrimidinyl or naphthyridinyl, each of which may be substituted with one or two substituent(s) selected from the group consisting of hydroxy, lower alkyl, lower alkoxy, halo(lower)alkyl, halogen, hydroxy(lower)alkyl, (lower)alkoxy(lower)alkyl, cyano, carboxy, (lower)alkoxycarbonyl, lower alkanoyl, carbamoyl, (mono or di)(lower)alkylcarbamoyl, [(lower)alkylsulfonyl]carbamoyl, amino, nitro, ureido, [(lower)alkylcarbonyl]amino, [(lower)alkylsulfonyl]amino and (arylsulfonyl)amino.

4. A compound of claim 3, wherein
X₁ is bond or —OCH₂—;
X₂ is —(CH₂)ₙ— in which n is 1;
X₃ is —O—;
R₁ is phenyl which may be substituted with one or two substituent(s) selected from the group consisting of halogen, nitro, amino, benzyloxy, benzyloxycarbonylamino, hydroxy and [(lower)alkylsulfonyl]amino; or pyridyl which may have amino.
R₂ is hydrogen.
R₃ is hydroxy(lower)alkyl; and
R₄ is pyridyl which may be substituted with carbamoyl, lower alkoxycarbonyl, carboxy, cyano, nitro, amino, hydroxy(lower)alkyl, mono(or di)(lower)-alkylcarbamoyl, lower alkyl, halogen, lower alkylsulfonylamino, phenylsulfonylamino or lower alkanoyl; phenyl which may be substituted with halogen; quinolyl which may be substituted with lower alkoxycarbonyl, nitro, carbamoyl, carboxy, halogen or lower alkoxy; naphthyl; benzothiazolyl; pyridyl N-oxide; pyrimidinyl; naphthyridinyl; pyrazinyl; imidazo[1,2-a]pyridyl; quinoxalinyl which may be substituted with halogen; acridinyl which may be substituted with halogen and lower alkoxy; or isoquinolyl which may be substituted with halogen;

5. A compound of claim 4, wherein
R¹ is phenyl which may be substituted with one or two substituent(s) selected from th group consisting of halogen, nitro, amino, benzyloxy, benzyloxycarbonylamino, hydroxy and lower alkylsulfonylamino.
R₄ is pyridyl which may be substituted with carbamoyl, lower alkoxycarbonyl, carboxy, cyano, nitro, amino, hydroxy(lower)alkyl, mono(or di)(lower)-alkylcarbamoyl, lower alkyl, halogen, lower alkylsulfonylamino, phenylsulfonylamino or lower alkanoyl; phenyl which may be substituted with halogen; quinolyl which may be substituted with lower alkoxycarbonyl, nitro, carbamoyl, carboxy, halogen or lower alkoxy; naphthyl; benzothiazolyl; pyridyl N-oxide; pyrimidinyl; naphthyridinyl; pyrazinyl; imidazo[1,2-a]pyridyl; quinoxalinyl which may be substituted with halogen; acridinyl which may be substituted with halogen and lower alkoxy; or isoquinolyl which may be substituted with halogen;

6. A compound of claim 4, wherein
R₁ is pyridyl which may have amino; and
R₄ is pyridyl which may have hydroxy(lower)alkyl.

7. A process for preparing a compound of claim 1,
or a salt thereof,
which comprises,
(i) reacting a compound [II] of the formula 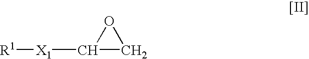
wherein X₁ and R¹ are each as defined in claim 1, with a compound [III] of the formula 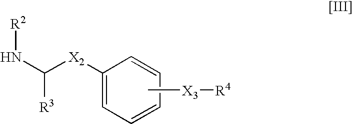
wherein X₂, X₃, R², R³ and R⁴ are each as defined in claim 1,
or a salt thereof, to give a compound [I] of the formula 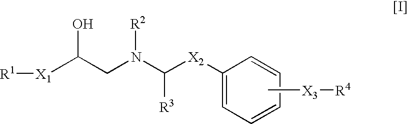
wherein X₁, X₂, X₃, R¹, R², R³ and R⁴ are each as defined in claim 1,
or a salt thereof,
(ii) subjecting a compound [Ia] of the formula 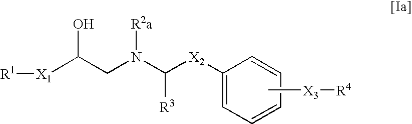
wherein X₁, X₂, X₃, R¹, R³ and R⁴ are each as defined in claim 1, and
Rₐ² is amino protective group,
or a salt thereof, to elimination reaction of the amino protective group, to give a compound [Ib] of the formula 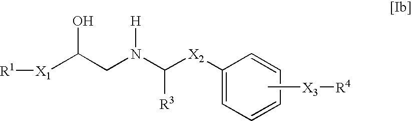
wherein X₁, X₂, X₃, R¹, R³ and R⁴ are each as defined in claim 1,
or a salt thereof,
(iii) reacting a compound [III] of the formula: 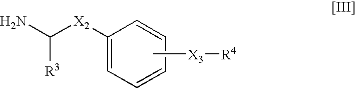
wherein X₂, X₃, R³ and R⁴ are each as defined in claim 1,
or a salt thereof with a compound [IV] of the formula: 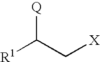
wherein R¹ is as defined in claim 1,
Q is protected hydroxy and
X is halogen,
to give a compound [Ic] of the formula: 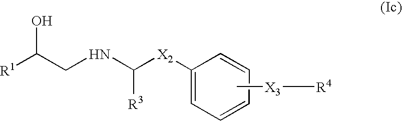
wherein X₂, X₃, R¹, R³ and R⁴ are each as defined in claim 1,
or a salt thereof, or
(iv) reacting a compound [V] of the formula: 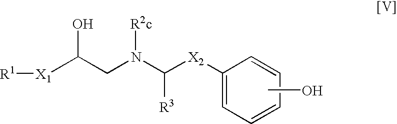
wherein X₁, X², R¹ and R³ are each as defined in claim 1, and R_{c}² is benzyl
with a compound [VI] of the formula: 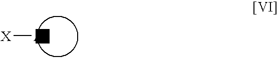
wherein 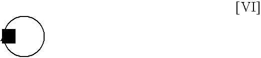
is aryl or an unsaturated heterocyclic group containing nitrogen, each of which may be substituted with one or two substituent(s) selected from the group consisting of hydroxy, lower alkyl, lower alkoxy, halo(lower)alkyl, halogen, hydroxy(lower)alkyl, (lower)alkoxycarbonyl, lower alkanoyl, carbamoyl, (mono or di)(lower)alkylcarbamoyl, [(lower)alkylsulfonyl]carbamoyl, amino, nitro, ureido, [(lower)alkylcarbamoyl]amino, [(lower)alkylsulfonyl]amino and (arylsulfonyl)amino, and
X is as defined above,
to give a compound [Id] of the formula: 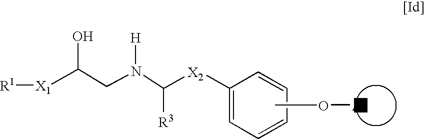
wherein X₁, X₂, R¹, R² and R³ are each as defined in claim 1, and 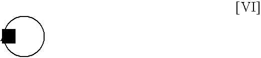
is as defined above,
or a salt thereof.

8. A pharmaceutical composition which comprises, as an active ingredient, a compound of claim 1 or a pharmaceutically acceptable salt thereof in admixture with pharmaceutically acceptable carriers or excipients.

9. Use of a compound of claim 1 or a pharmaceutically acceptable salt thereof for the manufacture of a medicament.

10. A compound of claim 1 or a pharmaceutically acceptable salt thereof for use as a medicament.

11. A compound of claim 1 or a pharmaceutically acceptable salt thereof for use as selective β₃ adrenergic receptor agonists.

12. A method for the prophylactic and/or the therapeutic treatment of pollakiuria or urinary incontinence which comprises administering a compound of claim 1 or a pharmaceutically acceptable salt thereof to a human being or an animal.
